(19) United States
(12) Patent Application Publication
(10) Pub. No.: US 2022/0017433 A1
(43) Pub. Date: Jan. 20, 2022

(54) PHOTOCHEMICALLY INDUCED CONJUGATION OF RADIOMETALS TO SMALL MOLECULES, PEPTIDES AND NANOPARTICLES IN A SIMULTANEOUS ONE-POT REACTION

(71) Applicant: UNIVERSITÄT ZÜRICH, Zürich (CH)

(72) Inventors: Jason Philip HOLLAND, Eglisau (CH); Malay PATRA, Zurich (CH)

(73) Assignee: UNIVERSITÄT ZÜRICH, Zürich (CH)

(21) Appl. No.: 17/295,457

(22) PCT Filed: Nov. 21, 2019
(86) PCT No.: PCT/EP2019/082159

§ 371 Date: May 20, 2021

(30) Foreign Application Priority Data
Nov. 21, 2018 (EP) 18207670.3
Nov. 21, 2018 (EP) 18207671.1

(51) Int. Cl. C07B 59/00; A61K 51/04

(52) U.S. Cl. CPC C07B 59/004; A61K 51/0468

(57) ABSTRACT

The invention relates to a method for labeling a target compound with a radiometal by photochemically induced conjugation. Furthermore, a chelating compound for use in said method is provided. The chelating compound is characterized by an arylazide moiety which can be photo-conjugated to a target compound and a chelator moiety which can be radiolabelled. The photo-conjugation and radiolabelling are both performed at basic pH performed in a simultaneous one-pot reaction.

The present invention relates to means and methods to label a target compound with a radiometal by photochemically induced conjugation.

### INTRODUCTION

The use of photochemically activated reagents for labelling proteins and biologically active molecules was introduced by Westheimer and co-workers in 1962. Since then, photoaffinity labelling (PAL) tools have matured, and a wide array of reagents are available for studying the structure and function of biological systems. Photochemical activation offers a number of advantages over thermochemical processes. For instance, photoreactive groups can be selected whereby, i) the reagent is stable under ambient conditions, ii) the photoactivation step occurs specifically at a wavelength that is not absorbed by the biological vector, and iii) the conjugation step involves a chemoselective reaction with target molecule. Further, since photochemical activation proceeds via an excited electronic state that typically leads to the formation of extremely reactive intermediates like carbenes, nitrenes and radicals, the rates of photochemical conjugation reactions can be several orders of magnitude faster than standard methods. High reactivity of the photo-induced intermediates presents both advantages and disadvantages. One of the benefits is that photoactive reagents can yield high labelling efficiencies in short reaction times. However, to achieve efficient conjugation, PAL methods often rely on a mechanism in which the photoactive reagent and the target protein form a non-covalent pre-association complex. Pre-association facilitates pseudo-first-order intramolecular bond formation, and minimises the probability of quenching by background medium (by the solvent, oxygen, salts etc). The problem with this approach is that it restricts most PAL tools to systems that self-assemble.

Photochemical reactions are an attractive platform for developing radiolabelled compounds. For molecules that undergo radioactive decay, chemical kinetics is one of the main factors in determining if a reaction is suitable for use in radiotracer synthesis. Since photochemical reactions often proceed with rate constants that tend toward the rate of diffusion, it is possible to combine photochemistry with radiochemistry (photoradiochemistry).

It is surprising that to date, photochemistry has had minimal impact in radiopharmaceutical science. The main bottlenecks to a more wide-spread use of photoradiochemistry for labelling proteins, peptides and small-molecules are, i) avoiding the need to form a pre-associated complex, ii) controlling chemoselectivity in the presence of competing nucleophiles, and ii) ensuring that the rate of productive bimolecular conjugation exceeds that of background quenching reactions. If a photochemical process can be tuned to facilitate bimolecular coupling, it is conceivable that photoradiochemistry may become a general tool in radiotracer synthesis.

A specific area for potential applications of photoradiochemistry is the radiochemical synthesis of labelled biomolecules such as monoclonal antibodies (mAbs) or immunoglobulin fragments for use in positron emission tomography (immuno-PET) and radioimmunotherapy (RIT). Available methods for radiolabelling rely on a two-step procedure (upper and lower pathway in FIG. 8).

Initially, the biomolecule, e.g. antibody, is purified from a source, and then functionalised with a suitable metal ion binding chelate. After conjugation, the functionalised biomolecule is re-purified, validated and stored prior to future radiolabelling experiments. Although this two-step approach is highly successful, there are several major drawbacks. First, the conjugation chemistry is time-consuming and may involve multiple chemical transformations that risk compromising the biological integrity of the biomolecule. Second, for applications in the clinic, the conjugation chemistry should ideally be performed in accordance with current Good Manufacturing Processes (cGMP). Third, the conjugated biomolecule is a new molecular entity (NME) which may be subject to stringent testing. Finally, storage of the radiolabelling precursor raises concerns over the long-term chemical and biochemical stability.

In the present invention, we present the photoradiochemical synthesis of radioactively labelled target compounds obtained in a simultaneous one-pot reaction.

### DESCRIPTION

Based on the above-mentioned state of the art, the objective of the present invention is to provide means and methods to label a target compound with a radiometal by photochemically induced conjugation. This objective is attained by the subject-matter of the independent claims of the present specification.

According to a first aspect of the invention, a method for preparing a photoradiolabelled compound comprising
i. providing a reaction mixture comprising
   at least one chelating compound, and
   at least one target compound B comprising an amine and/or thiol, and/or carboxylate moiety, and
   at least one radioactive ion of a radionuclide,
ii. in a photoradiolabelling step,
   adjusting the pH to pH>7, in particular pH>8, more particularly pH 8 to 11,
   irradiation of the reaction mixture with light at a wavelength selected from 200 nm to 420 nm,
wherein the chelating compound is a compound of formula 1,

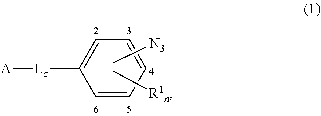

wherein
A is a chelator suitable for coordinating an ion of a radionuclide at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂—CHF₂, —SOCF₃, —SO₂CF₃, —SO₂—NR²R³, —CN, —NO₂, —F, —Cl, —Br or —I, in particular ——OH, —OR⁴, —CN, —NO₂, —F, —Cl, —Br, or —I, with
   R² and R³ being independently selected from C₁₋₆-alkyl, C₂₋₆-alkenyl and C₂-₆-alkynyl,
   R⁴ being selected from C₁₋₆-alkyl, C₂₋₆-alkenyl and C₂₋₆-alkynyl which may optionally be substituted with —F, —Cl, —Br or —I,
n is 0, 1, 2 or 3, in particular 0 or 1, more particularly 0, and

R¹ and —N₃ are positioned in such a way that at least one of the positions 2 to 6 of the phenyl moiety that are next to —N₃ is unsubstituted.

In certain embodiments, the method for preparing a photoradiolabelled compound comprises
i. providing a reaction mixture comprising
   at least one chelating compound, and
   at least one target compound B comprising an amine and/or thiol moiety, and
   at least one radioactive ion of a radionuclide,
ii. in a photoradiolabelling step,
   adjusting the pH to pH>7, in particular pH>8, more particularly pH 8 to
   irradiation of the reaction mixture with light at a wavelength selected from 200 nm to 420 nm,
wherein the chelating compound is a compound of formula 1,

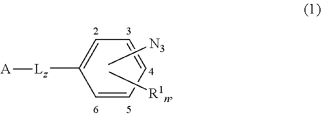

wherein
A is a chelator suitable for coordinating an ion of a radionuclide at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂-CHF₂, —SOCF₃, —SO₂CF₃, —SC₂—NR²R³, C—ON, —NO₂, —F, —Cl, —Br or —I, in particular ——OH, —OR⁴, —CN, —NO₂, —F, —Cl, —Br, or —I, with
   R² and R³ being independently selected from C₁₋₆-alkyl, C₂₋₆-alkenyl and C₂-C₆-alkynyl,
   R⁴ being selected from C₁₋₆-alkyl, C₂₋₆-alkenyl and C₂₋₆-alkynyl which may optionally be substituted with —F, —Cl, —Br or —I.

In certain embodiments, n is 0, 1, 2 or 3, in particular 0 or 1, more particularly 0, and R¹ and —N₃ are positioned in such a way that at least one of the positions 2 to 6 of the phenyl moiety that are next to —N₃ is unsubstituted.

The terms “radiation” and “irradiation” are used interchangeably in this specification.

The method according to the invention is directed towards simultaneous radiolabelling of a chelator moiety of a chelating compound and photoconjugation of an aryl-azide moiety of said chelating compound to a target compound. Irradiation of the aryl-azide releases N₂ forming a singlet arylnitrene, which at room temperature undergoes extremely fast intramolecular rearrangement to give ketenimines (or benzazirine) intermediates. Ketenimines react relatively slowly with oxygen, protons and water, but undergo rapid nucleophilic addition with amines or thiols of said target compound. The addition is facilitated if the amine, e.g. ε-NH₂ of lysine, or thiol moiety, e.g. —SH of cysteine, is deprotonated. Deprotonation is achieved by adjusting the pH to pH>7.

The simultaneous radiolabelling and photoconjugation is performed in a simultaneous one-pot reaction without any purification or isolation step between radiolabelling and photoconjugation. In the context of the present specification “simultaneous” means that radiolabelling and photoconjugation occur in the same experimental step. Thus, some compounds of formula 1 in the reaction mixture may first react via their aryl-azide moiety with a target compound and then coordinate a radionuclide, while other compounds of formula 1 first coordinate a radionuclide and then bind to a target compound or both reactions occur at the same time at one compound of formula 1. In this sense, i.e. taking the entirety of all compounds of formula 1 in the reaction mix into consideration, the radiolabelling and photoconjugation is performed simultaneously. The specific reaction sequence with regard to one specific compound of formula 1 in the reaction mixture depends for example on the local availability and orientation towards the reactive side of a target compound.

In certain embodiments, the photoconjugation and radiolabelling is performed simultaneously.

In certain embodiments, the photoradiolabelling step is performed without a purification step between photoconjugation and radiolabelling.

In certain embodiments, the photoradiolabelling step consists of adjusting the pH to pH>7, in particular pH>8, more particularly pH 8 to 11, and irradiation of the reaction mixture with light at a wavelength selected from 200 nm to 420 nm.

In certain embodiments, the target compound comprises a primary, secondary or tertiary amine and/or thiol moiety.

In certain embodiments, the target compound comprises a primary or secondary amine and/or thiol moiety.

In certain embodiments, the target compound comprises a primary or secondary amine —NHR^{h} and/or thiol moiety with R^{h} being a residue that does not react with the chelating compound under the reaction conditions of the method according to the invention.

In certain embodiments, the target compound comprises a primary or secondary amine (—NHR^{h}) and/or thiol moiety (—SH) with R^{h} being H or substituted or unsubstituted C₁₋₁₂-alkyl.

In certain embodiments, the target compound comprises a primary or secondary amine (—NHR^{h}) with R^{h} being H or C₁₋₁₂-alkyl, particularly C₁₋₆-alkyl.

In certain embodiments, the target compound comprises a cysteine and/or lysine.

In certain embodiments, the target compound comprises a lysine.

In contrast to known labelling methods, the method according to the invention is performed in a one-pot reaction. Usually, the method can be performed in less than one hour, particularly in less than 15 min. Under optimized conditions, the reaction is complete in <10 min.

For the radiolabelling, at least one ion of a radionuclide is required.

In certain embodiments, the radionuclide is selected from ⁴³Sc, ⁴⁴Sc, ⁴⁷Sc, ⁴⁵Ti, ⁵¹Cr, ⁵¹Mn, ⁵²Mn, ^{52m}Mn, ⁵²Fe, ⁵⁵Co, ⁵⁷Ni, ⁶⁰Cu, ⁶¹Cu, ⁶²Cu, ⁶⁴, ⁶⁷Cu, ⁶⁵Zn, ⁶⁶Ga, ⁶⁷Ga, ⁶⁸Ga, ⁶⁹Ge, ⁷¹As, ⁷²As, ⁷⁴As, ⁷⁶As, ⁷⁷As, ⁸²Rb , ^{82m}Rb, ⁸²Sr, ⁸³Sr, ⁸⁹Sr, ⁸⁶Y, ⁹⁰Y, ⁸⁹Zr, ⁹⁷Zr, ⁹⁰Nb, ^{94m}Tc, ^{99m}Tc, ⁹⁷Ru, ¹⁰⁵Rh, ¹¹¹Ag, ^{110m}In, ¹¹¹In, ^{117m}Sn, ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, ¹⁷⁸Ta, ¹⁸⁶Re, ¹⁸⁸Re, ¹⁹²Ir, ^{195m}Pt, ¹⁹⁸Au, ^{197m}Hg, ²⁰¹Tl, ²¹²Pb, ²¹²Bi, ²¹³Bi, ²¹¹At, ²²³Ra, ²⁵⁵Ac.

In certain embodiments, the radionuclide is selected from ⁴³Sc, ⁴⁴Sc, ⁴⁷Sc, ⁶⁰Cu, ⁶¹Cu, ⁶²Cu, ⁶⁴Cu, ⁶⁷Cu, ⁶⁷Ga, ⁶⁸Ga, ⁸⁶Y, ⁹⁰Y, ⁸⁹Zr, ^{99m}Tc, ¹¹¹In, ¹⁵³Sm, ¹⁴⁹Tb, ¹⁵²Tb, ¹⁵⁵Tb, ¹⁶¹Tb, ⁷⁷Lu, ¹⁸⁶Re, ¹⁸⁸Re, ²¹²Pb, ²¹²Bi, ²¹³Bi, ²²³Ra, ²⁵⁵Ac.

In certain embodiments, the radionuclide is selected from ⁶⁸Ga, ⁸⁹Zr, ⁶⁴Cu, ⁶⁷Cu ⁹⁰Y, ^{99m}Tc, ¹⁷⁷Lu, ²¹²Pb, ²¹²Bi, ²¹³Bi ²²³Ra, ²²⁵Ac.

In certain embodiments, the radionuclide is ⁸⁹Zr.

For the method according to the invention, the radioactive ion has to be soluble under basic conditions. To enhance the solubility and to stabilize the radioactive ion at basic pH, suitable co-ligands may be added.

In certain embodiments, a co-ligand is added to the reaction mixture.

In certain embodiments, acetate, oxalate or chloride is added to the reaction mixture.

The method according to the invention can be performed using one type of chelating compound, for example a chelating compound comprising the chelator Desferrioxamine B (DFO), and one type of radioactive ion, e.g. ⁸⁹Zr.

According to a second aspect of the invention, a chelating compound is provided. The chelating compound comprises formula 2,

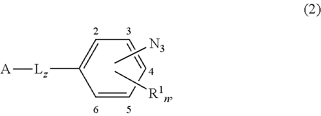

wherein
A is a chelator suitable for coordinating an ion of a radionuclide, particularly at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂—CHF₂, —SOCF₃, —SO₂CF₃, —SO₂—NR²R³, —CN, —NO₂, —F, —Cl, —Br or —I, in particular ——OH, —OR⁴, —CN, —NO₂, —F, —Cl, —Br, or —I, with
   R² and R³ being independently selected from C₁₋₆-alkyl, C₂₋₆-alkenyl and C₂₋₆-alkynyl,
   R⁴ being selected from C₁₋₆-alkyl, C₂₋₆-alkenyl and C₂₋₆-alkynyl which may optionally be substituted with —F, —Cl, —Br or —I,
n is 0, 1, 2 or 3, in particular 0 or 1, more particularly 0,
   wherein R¹ and —N₃ are positioned in such a way that at least one of the positions 2 to 6 of the phenyl moiety that are next to —N₃ is unsubstituted,
with the proviso that in case of z being 0, A is not EDTA, and
with the proviso that in case of z being 1, A is not DTPA.

In certain embodiments, A is a chelator suitable for coordinating an ion or a radionuclide at basic pH.

In certain embodiments, the chelating compound comprises formula 2,

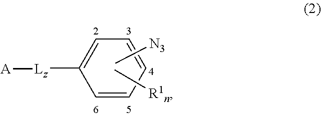

wherein
A is a chelator suitable for coordinating an ion of a radionuclide at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CH F₂, —CH₂—CF₃, —CH₂—CH₂F, —CH₂—CHF₂, —SOCF₃, —SO₂CF₃, —SC₂—NR²R³, —CN, —NO₂, —F, —Cl, —Br or —I, in particular ——OH, —OR⁴, —CN, —NO₂, —F, —Cl, —Br, or —I, with
   R² and R³ being independently selected from C₁₋₆-alkyl, C₂₋₆-alkenyl and C₂-₆-alkynyl,
   R⁴ being selected from C₁₋₆-alkyl, C₂₋₆-alkenyl and C₂₋₆-alkynyl which may optionally be substituted with —F, —Cl, —Br or —I,
n is 0, 1, 2 or 3, in particular 0 or 1, more particularly 0,
   wherein R¹ and —N₃ are positioned in such a way that at least one of the positions 2 to 6 of the phenyl moiety that are next to -N₃ is unsubstituted,
with the proviso that in case of z being 0, A is not EDTA, DTPA, MA-DTPA, CA, TETA, DOTA or DADS, and
with the proviso that in case of z being 1, A is not DTPA.

In certain embodiments of all aspects of the invention, R⁴ is selected from C₁₋₆-alkyl.

In certain embodiments of all aspects of the invention, R⁴ is selected from C₁₋₃-alkyl.

In certain embodiments of all aspects of the invention, R⁴ is methyl or ethyl.

According to a third aspect, a radiolabelled intermediate compound is provided. The radiolabelled intermediate compound comprises formula 3,

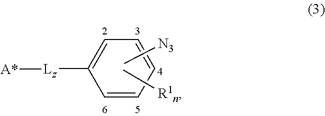

wherein

A* is a chelator bound to a radionuclide by coordinate bonds, and L, z, R¹ and n are defined as described above.

The radiolabelled intermediate compound occurs if the chelator moiety A of the chelating compound coordinates first a radioactive ion. Subsequently, the radiolabelled intermediate compound undergoes nucleophilic addition to an amine or thiol, particularly to an amine moiety, of a target compound B induced by the irradiation.

In certain embodiments of the first, second and third aspect of the invention, —N₃ is in meta or para position, particularly in para position.

According to a fourth aspect of the invention, a photoconjugated intermediate compound is provided. The photoconjugated intermediate compound comprises formula 4a, 4b, 4c, 4d or 4e, in particular 4a, 4b or 4c, more particularly 4a,

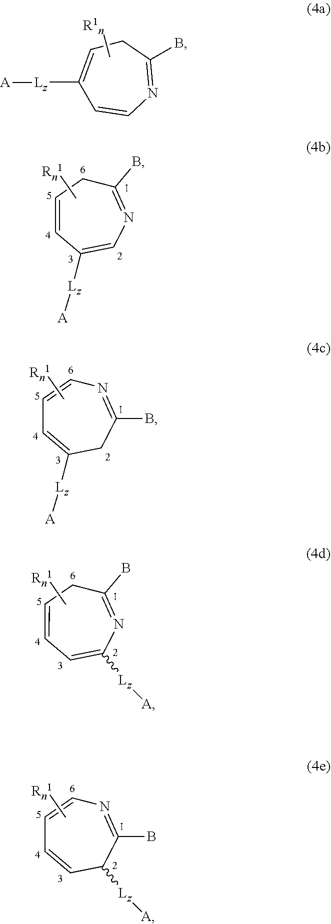

wherein A, L, z, R¹, n and B are defined as described above.

The photoconjugated intermediate compound occurs if the chelating compound undergoes first nucleophilic addition induced by the irradiation. Subsequently, the photoconjugated intermediate compound is labelled with a radioactive ion.

The photoconjugated intermediate compound of formula 4a occurs if a chelating compound of formula 3 with —N₃ being in para position (position 4 in formula 3) reacts with a target compound B.

The photoconjugated intermediate compound of formula 4b or 4c occurs if a chelating compound of formula 3 with —N₃ being in meta position (position 3 or 5 in formula 3) reacts with a target compound B (Scheme A).

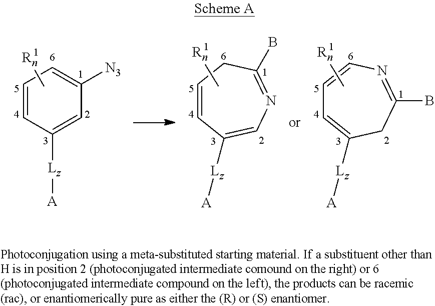

The photoconjugated intermediate compound of formula 4d or 4e occurs if a chelating compound of formula 3 with —N₃ being in ortho position (position 2 or 6 in formula 3) reacts with a target compound B (Scheme B).

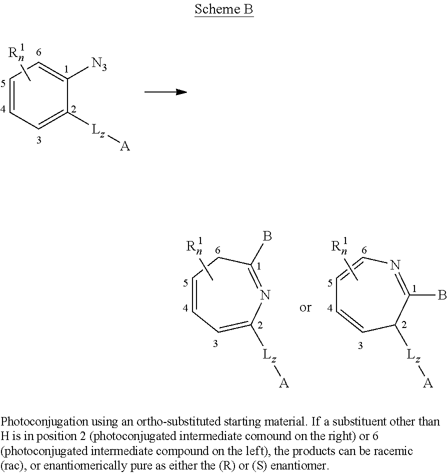

According to a fifth aspect of the invention, a photoradiolabelled compound is provided. The photoradiolabelled compound comprises formula 5a, 5b, 5c, 5d or 5e, in particular 5a, 5b or 5c, more particularly 5a,

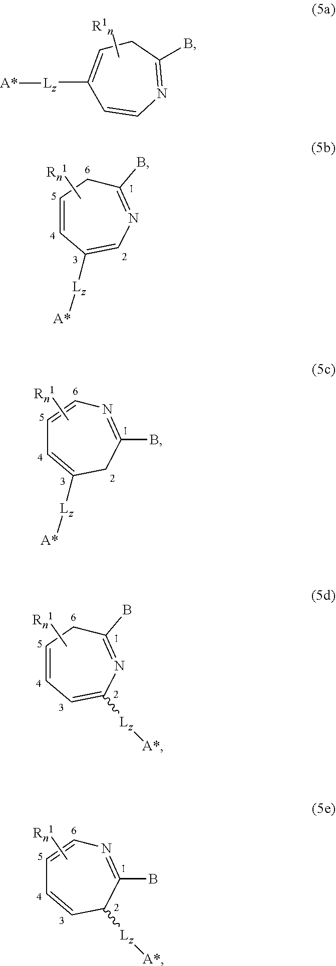

wherein A*, L, z, R¹, n and B are defined as described above.

The photoradiolabelled compound of formula 5a occurs if a chelating compound of formula 3 with —N₃ being in para position (position 4 in formula 3) was used in the method according to the invention.

The photoradiolabelled compound of formula 5b or 5c occurs if a chelating compound of formula 3 with -N₃ being in meta position (position 3 or 5 in formula 3) was used in the method according to the invention. If a substituent other than H is in position 2 (formula 5c) or 6 (formula 5b), the photoradiolabelled compound can be racemic (rac), or enantiomerically pure as either the (R) or (S) enantiomer.

The photoradiolabelled compound of formula 5d or 5e occurs if a chelating compound of formula 3 with -N₃ being in ortho position (position 2 or 6 in formula 3) was used in the method according to the invention. If a substituent other than H is in position 2 (formula 5e) or 6 (formula 5d), the photoradiolabelled compound can be racemic (rac), or enantiomerically pure as either the (R) or (S) enantiomer.

In certain embodiments of all aspects of the invention, the chelator is selected from NODAGA, NOTA, DOTA, Desferrioxamine B (DFO), ATSM, DOTAGA, HBED-CC, SAAC, DTPA, DTPA-benzyl, DFO-Star, oxoDFO-Star, HOPO, p-SCN-Bn-HOPO,

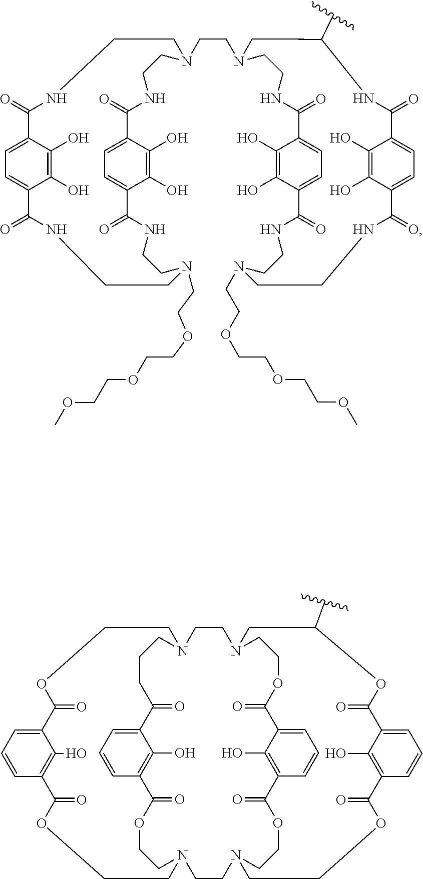 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)CCCN(CCOC(=O)c3cccc(c3O)C(=O)OCC1)CC2]

and derivatives thereof.

In certain embodiments of all aspects of the invention, the chelator is selected from NODAGA, NOTA, DOTA, Desferrioxamine B (DFO), ATSM, DOTAGA, HBED-CC, SAAC, DTPA, DTPA-benzyl, DFO-Star, oxoDFO-Star, HOPO, p-SCN-Bn-HOPO,

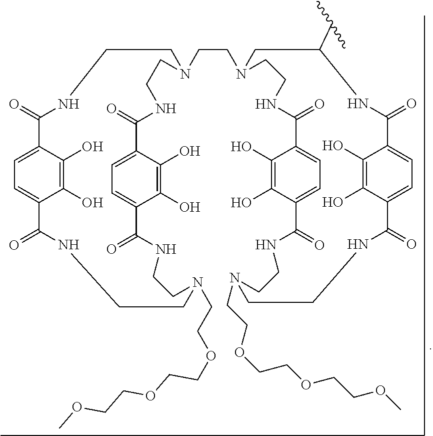 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1]

In certain embodiments of all aspects of the invention, the chelator is selected from NODAGA, NOTA, DOTA, Desferrioxamine B (DFO), ATSM, DOTAGA, HBED-CC, SAAC, DTPA, DTPA-benzyl, DFO-Star, oxoDFO-Star, p-SCN-Bn-HOPO,

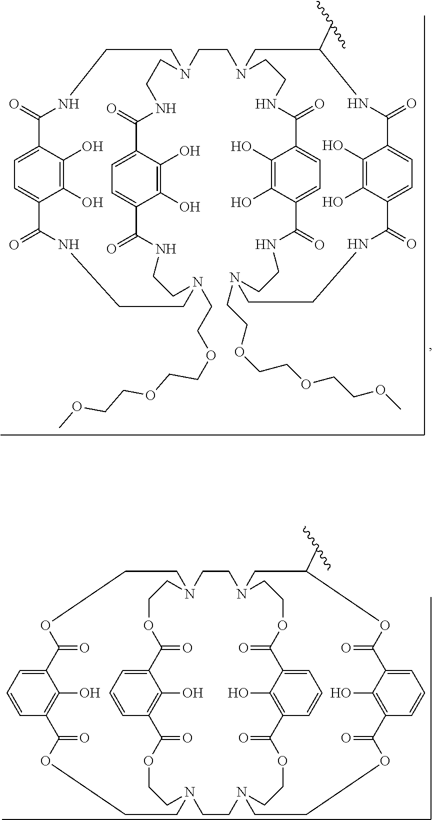 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]

and derivatives thereof. If the chelator is DTPA, the chelating molecule does not comprise a linker.

Usually, the chelator used is not coordinated to a metal ion. However, it is also possible to use a chelator coordinated to a non-radioactive metal ion, e.g. ZnATSM/en, whereby the Zn ion is subsequently exchanged by a radioactive ion by transmetallation.

In certain embodiments of all aspects of the invention, the chelator is selected from NODAGA, NOTA, DOTA, Desferrioxamine B (DFO), ATSM, DOTAGA, HBED-CC, SAAC, DTPA-benzyl, DFO-Star, oxoDFO-Star, p-SCN-Bn-HOPO,

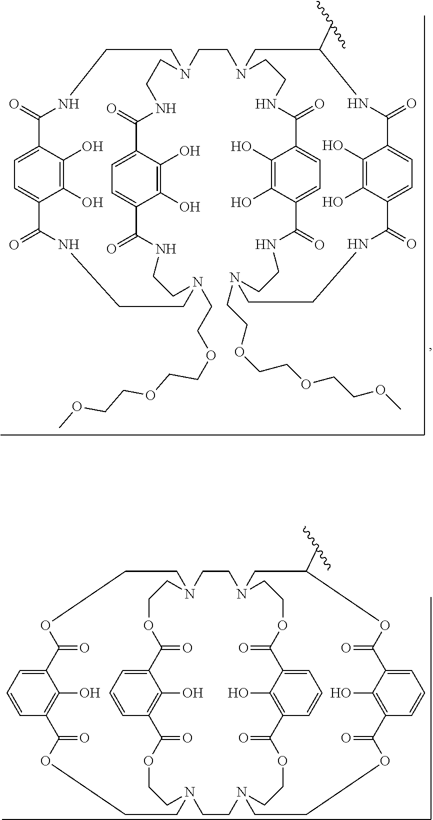 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]

and derivatives thereof.

In certain embodiments of all aspects of the invention, the chelator is selected from NODAGA, NOTA, Desferrioxamine B (DFO), ATSM, DOTAGA, HBED-CC, SAAC, DFO-Star, oxoDFO-Star, p-SCN-Bn-HOPO,

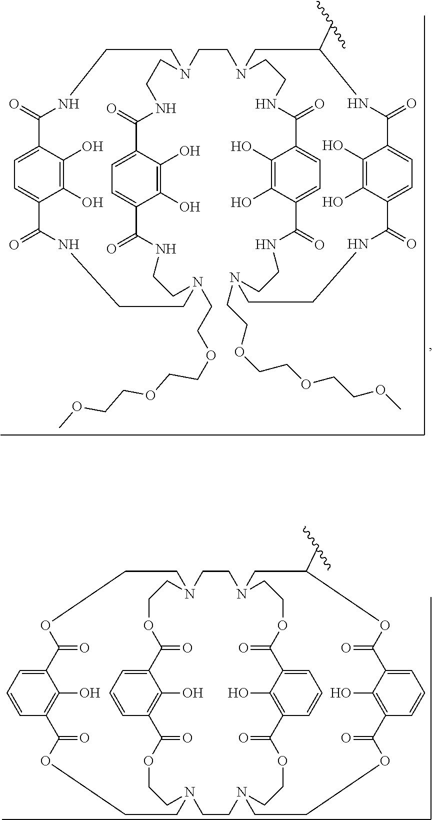 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]

and derivatives thereof.

In certain embodiments of all aspects of the invention, the chelator is selected from Desferrioxamine B (DFO), DFO-Star, oxoDFO-Star and derivatives thereof.

In certain embodiments of all aspects of the invention, the chelator is selected from Desferrioxamine B (DFO), DFO-Star, oxoDFO-Star.

In certain embodiments of the first, third or fifth aspect of the invention,
the chelator moiety A is selected from NOTA, NODAGA, DOTA and DOTAGA and and the radioactive ion is of a radionuclide selected from ⁶⁶Ga, ⁶⁷Ga, ⁶⁸Ga, ⁶⁰Cu, ⁶¹Cu, ⁶²Cu, ⁶⁴Cu, ⁶⁷Cu, ⁴³Sc, ⁴⁴Sc and ⁴⁷Sc, or
the chelator moiety A is selected from DOTA and DOTAGA and the radioactive ion is of a radionuclide is selected from ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, or
the chelator moiety A is selected from DFO (desferrioxamine B), DFOstar, oxDFO-star, HOPO,

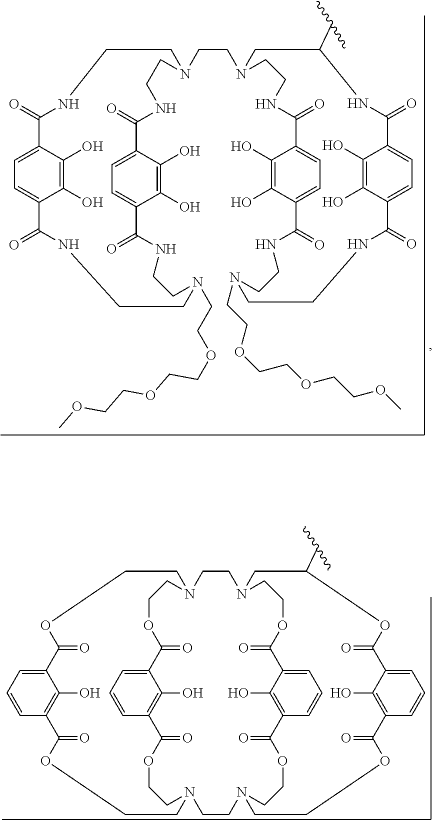 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]

and the radioactive ion of a radionuclide is selected from ⁸⁹Zr, ⁹⁷Zr, ⁶⁶Ga, ⁶⁷Ga, ⁶⁸Ga, or
the chelator moiety A is ATSM and the radioactive ion of a radionuclide is selected from ⁶⁰Cu, ⁶¹Cu, ⁶²Cu, ⁶⁴Cu, ⁶⁷Cu,
the chelator moiety A is SAAC and the radioactive ion of a radionuclide is selected from ^{99m}Tc, ¹⁸⁶Re, ¹⁸⁸Re,
the chelator moiety A is HBED-CC and the radioactive ion of a radionuclide is selected from ⁶⁶Ga, ⁶⁷Ga, ⁶⁸Ga, ^{110m}In, ¹¹¹In (Table A).

TABLE A
Suitable combinations of chelator moieties A and radionuclides

| | Radionuclides |
|---|---|
| Chelator moiety A | of elements |
| NOTA, NODAGA, DOTA and DOTAGA, | Ga, Cu, Sc |
| DOTA and DOTAGA | lanthanides and |
| | actinides |
| DFO (desferrioxamine B), DFOstar, oxDFO-star, HOPO, 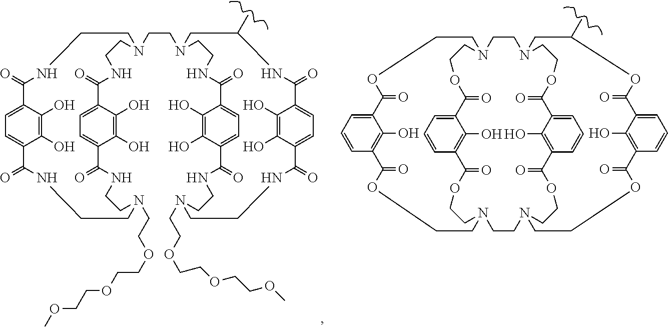 | Zr and Ga |
| ATSM | Cu |
| SAAC | ^{99m}Tc, Re |
| HBED-CC | Ga, In |

In certain embodiments of all aspects of the invention, L is a linker comprising one or more moieties, particularly 1 to 20 moieties, more particularly 1 to 15 moieties, selected from —C(═X)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, —O—, —C₁₋₈-alkyl-, with R⁶ being H or C₁₋₆-alkyl and X being O or S.

In certain embodiments of all aspects of the invention, L is a linker comprising one or more moieties, particularly 1 to 20 moieties, more particularly 1 to 15 moieties, selected from —C(═X)—, —NR⁶—, —C(═X)—NR⁶—, —NR⁶—C(=X)—, —NR⁶—C(═X)—NR⁶—, —O—, —C₁₋₈-alkyl-, with R⁶ being H or C₁₋₆-alkyl and X being O or S.

In certain embodiments of all aspects of the invention, L is —C(═O)— or L comprises one or more moieties selected from —C(═X)—, —NR⁶—, —C(═X)−NR⁶—, —NR⁶—C(═X)—, —NR⁶—C(═X)—NR⁶—, —O—, —C₁₋₈-alkyl- with R⁶ being H or C₁₋₆-alkyl and X being O or S, wherein a moiety that comprises a heteroatom N, O or S alternates with an alkyl moiety.

In certain embodiments of all aspects of the invention, L is —C(═O)—or L comprises one or more moieties selected from —C(═X)—, —NR⁶—, —C(═X)—NR⁶—, —NR⁶—C(═X)—, —NR⁶—C(═X)—NR⁶—, —O—, —C₁₋₈-alkyl- with R⁶ being H or C₁₋₆-alkyl and X being O or S, wherein a moiety that comprises a heteroatom N, O or S alternates with an alkyl moiety, and wherein one or both ends of the linker are independently formed by a moiety that comprises a heteroatom N, O or S. For example, a linker —NH—C₃H₆—O—C₂H₄—O—C₂H₄—O—C₃H₆—NH—C(═O)— starts and ends with a moiety comprising a heteroatom N, O or S (—NH— and —NH—C(═O)—). Furthermore, there are alternating alkyl and heteroatom moieties: —NH—C₃H₆—O—C₂H₄—O—C₂H₄—O—C₃H₆ (moieties comprising a heteroatom are underlined).

In certain embodiments, L is —C(═O)— or a moiety of formula 2, —R^{a}ₙ—(C₁₋₆-alkyl)-R^{b}ₘ—R^{c}— (2), wherein

In certain embodiments of all aspects of the invention, R^{a} is —C(═O)—, —NR⁶—C(═X)—NR⁶—, or —NR⁶—, with R⁶ being H or C₁₋₄-alkyl, or R^{a} is a moiety —X¹—C₁₋₆-alkyl —X²— with X¹ and X² being a moiety independently selected from —C(═O)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, particularly —O—(═O)—, —NR⁶—, —C(═O)—NR⁶—, —NR⁶—C(═O)—, n is 0 or 1, R^{b} is a polyether moiety with p elements [—O—Cᵤ-alkyl], wherein u is independently selected for each element from an integer between 1 to 4 and p is an integer between 1 and 6, m is 0 or 1, R^{c} is —NR⁵—C(═O)—, —NR⁵—C(═X)—NR⁵—, —O—C(═X)—NR⁵—, —NR⁵—C(═X)—O—, wherein R⁵ is independently from each other H or C₁₄-alkyl, X is O or S, particularly S.

In certain embodiments of all aspects of the invention, R^{a} is —C(═O)— or —NR⁶— with R⁶ being H or C₁₄-alkyl, or R^{a} is a moiety —X¹—C₁₋₆-alkyl —X²— with X¹ and X² being a moiety independently selected from —C(═O)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, particularly —C(═O)—, —NR⁶—, —C(═O)—NR⁶—, —NR⁶—C(═O)—, n is 0 or 1, R^{b} is a polyether moiety with p elements [—O—Cᵤ-alkyl], wherein u is independently selected for each element from an integer between 1 to 4 and p is an integer between 1 and 6, m is 0 or 1, R^{c} is —NR⁵—C(═O)—, —NR⁵—C(═X)—NR⁵—, —O—C(═X)—NR⁵—, —NR⁵—C(═X)—O—, wherein R⁵ is independently from each other H or C₁₋₄-alkyl X is O or S, particularly S.

In certain embodiments of all aspects of the invention, R^{a} —NR⁶— with R⁶ being H or C₁₋₄-alkyl, or R^{a} is a moiety —X¹—C₁₋₆-alkyl —X²— with X¹ and X² being a moiety independently selected from —C(═O)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, particularly —C(═O)—, —NR⁶—, —C(═O)—NR⁶—, —NR⁶—C(═O)—, n is 0 or 1, R^{b} is a polyether moiety with p elements [—O—Cᵤ-alkyl], wherein u is independently selected for each element from an integer between 1 to 4 and p is an integer between 1 and 6, m is 0 or 1, R^{c} is —NR⁵—C(═O)—, —NR⁵—C(═X)—NR⁵—, —O—C(═X)—NR⁵—, —NR⁵—C(═X)—O—, wherein R⁵ is independently from each other H or C₁₋₄-alkyl, X is O or S, particularly S.

In certain embodiments of all aspects of the invention, R^{a} is —C(═O)—, —NR⁶—C(═S)—NR⁶—, or —NR⁶—, particularly —C(═O)— or —NR⁶—, more particularly —NR⁶—, with R⁶ being H or C₁₋₄-alkyl, or R^{a} is a moiety —X¹—C₁₋₆-alkyl —X²— with X¹ and X² being a moiety independently selected from —C(═O)—, —NR⁶—, —C(═O)—NR⁶—, —NR⁶—C(═O)—, —NR⁶—C(═S)—NR⁶—, —O—C(═O)—NR⁶—, —NR⁶—C(═O)—O—, particularly —C(═O)—, —NR⁶—, —C(═O)—NR⁶—, —NR⁶—C(═O)—.

In certain embodiments of all aspects of the invention, the linker L is —C(═O)— or a moiety of formula 2,
—R^{a}ₙ—(C₁₋₆-alkyl)-R^{b}ₘ—R^{c}_R^{d}ₜ (2), wherein
   R^{a} is —NR⁶— with R⁶ being H or C₁₋₄-alkyl, n is 0 or 1,
   R^{b} is a polyether moiety with p elements [—O—Cᵤ-alkyl], wherein u is independently selected for each element from an integer between 1 to 4 and p is an integer between 1 and 6,
   m is 0 or 1,
   R^{c} is —NR⁵—C(═O)—, —NR⁵—C(═X)—NR⁵—, —O—C(═X)—NR⁵—, —NR⁵—C(═X)—O—, wherein
      R⁵ is independently from each other H or C₁₋₄-alkyl,
      X is O or S, particularly S
   R^{d} is a C₁₋₆-alkyl,
   t is 0 or 1.

In certain embodiments of all aspects of the invention, the linker L is —C(═O)— or a moiety of formula 2,
—R^{a}ₙ—(C₁₋₆-alkyl)-R^{b}ₘ—R^{c} (2), wherein
   R^{a} is —NR⁶— with R⁶ being H or C₁₋₄-alkyl,
   n is 0 or 1,
   R^{b} is a polyether moiety with p elements [—O—Cᵤ-alkyl], wherein u is independently selected for each element from an integer between 1 to 4 and p is an integer between 1 and 6,
   m is 0 or 1,
   R^{c} is —NR⁵—C(═O)—, —NR⁵—C(═X)—NR⁵—, —O—C(═X)—NR⁵—, —NR⁵—C(═X)—O—, wherein
      R⁵ is independently from each other H or C₁₋₄-alkyl,
      X is O or S, particularly S.

In certain embodiments of all aspects of the invention, the linker L is —C(═O)— or a moiety of formula 2,
—R^{a}ₙ—(C₁₋₆-alkyl)-R^{b}ₘ—R^{c} (2), wherein
   R^{a} is —NR⁶— with R⁶ being H or C₁₋₄-alkyl,
   n is 0 or 1,
   R^{b} is a polyether moiety with p elements [—O—Cᵤ-alkyl], wherein u is independently selected for each element from an integer between 1 to 4 and p is an integer between 1 and 6,
   m is 0 or 1,
   R^{c} is —NR⁵—C(═O)—, wherein
      R⁵ is independently from each other H or C₁₄-alkyl.

Linkers comprising a polyether moiety R^{b} contribute to the solubility of the chelating compound. If the chelator A is poorly soluble in an aqueous solution, a linker comprising R^{b} might be chosen for the chelating compound.

In certain embodiments of the third or fifth aspect of the invention, the radionuclide is selected from ⁴³Sc, ⁴⁴Sc, ⁴⁷Sc, ⁴⁵Ti, ⁵¹Cr, ⁵¹Mn, ⁵²Mn, ^{52m}Mn, ⁵²Fe, ⁵⁵Co, ⁵⁷Ni, ⁶⁰Cu, ⁶¹Cu, ⁶²Cu, ⁶⁴Cu, ⁶⁷Cu, ⁶⁵Zn, ⁶⁶Ga, ⁶⁷Ga, ⁶⁸Ga, ⁶⁹Ge, ⁷¹As, ⁷²As, ⁷⁴As, ⁷⁶ As, ⁷⁷As, ⁸²Rb ^{82m}Rb, ⁸²Sr, ⁸³Sr, ⁸⁹Sr, ⁸⁶Y, ⁹⁰Y, ⁸⁹Zr, ⁹⁷Zr, ⁹⁰Nb, ^{94m}Tc, ^{99m}Tc, ⁹⁷Ru, ¹⁰⁵Rh, ¹¹¹Ag, ^{110m}In, ¹¹¹¹_{In,} ^{117m}Sn, ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, ¹⁷⁸Ta, ¹⁸⁶Re, ¹⁸⁸Re, ¹⁹²Ir, ^{195m}Pt, ¹⁹⁸Au, ^{197m}Hg, ²⁰¹Tl, ²¹²Pb, ²¹²Bi, ²¹³Bi, ²¹¹At, ²²³Ra, ²⁵⁵Ac.

In certain embodiments of the third or fifth aspect of the invention, the radionuclide is selected from 43Sc, ⁴⁴Sc, ⁴⁷Sc, ⁶⁰Cu, ⁶¹Cu, ⁶²Cu, ⁶⁴Cu, ⁶⁷C u, ⁶⁷Ga, ⁶⁸Ga, ⁸⁶Y, ⁹⁰Y, ⁸⁹Zr, ^{99m}Tc, ¹¹¹In, ¹⁵³Sm, ¹⁴⁹Tb, ¹⁵²Tb, ¹⁵⁵Tb, ¹⁶¹Tb, ⁷⁷Lu, ¹⁸⁶Re, ¹⁸⁸Re, ²¹²Pb, ²¹²Bi, ²¹³Bi, ²²³Ra, ²⁵⁵Ac.

In certain embodiments of the third or fifth aspect of the invention, the radionuclide is selected from ⁶⁸Ga, ⁸⁹Zr, ⁶⁴Cu, ⁶⁷Cu ⁹⁰Y, ^{99m}Tc, ¹⁷⁷Lu, ²¹²Pb, ²²⁵Ac.

In certain embodiments of the third or fifth aspect of the invention, the radionuclide is ⁸⁹Zr.

In certain embodiments of the first, fourth or fifth aspect of the invention, the target compound B is selected from a small molecule, a peptide, a protein, an antibody, an antibody-like molecule, an antibody fragment or a nanoparticle.

In certain embodiments of the first, fourth or fifth aspect of the invention, the target compound B is selected from a peptide, a protein, an antibody, an antibody-like molecule or an antibody fragment. In certain embodiments, the antibody is an IgG1 antibody, the antibody-like molecule is an IgG1-antibody-like molecule and the antibody fragment is an IfG1 antibody fragment. In certain embodiments, the antibody is an IgG1 antibody.

The photoradiolabelled compound may be used in positron emission tomography (immune-PET) or radioimmunotherapy (RIT). If the target molecule is an antibody or antibody fragment, e.g. an IgG1 antibody, that bind to specific marker molecules, e.g. the epidermal growth factor receptor HER2/neu, on the surface of cancer cells, the photoradiolabelled compound may be a useful tool in the diagnosis of specific diseases, e.g. cancer.

In certain embodiments, the target compound B is bound to the azepine moiety via said amine of the target compound B or a thioether moiety —S— derived from the thiol moiety —SH of the target compound B.

In certain embodiments, target compound B is bound to the azepine moiety via a secondary or tertiary amine —NR^{h}— derived from the primary or secondary amine of the target compound

B with R^{h} being H, C₁₋₁₂-alkyl, in particular C₁₋₆-alkyl, or a thioether moiety —S— of the target compound B.

In certain embodiments, target compound B is bound to the azepine moiety via a secondary or tertiary amine —NR^{h}— derived from the primary or secondary amine of the target compound B with R^{h} being H, C₁₋₁₂-alkyl, in particular C₁₋₆-alkyl.

In certain embodiments, the target compound B is bound to the azepine moiety via an amine —NH— derived from the ε-NH₂ of lysine or the —SH moiety of cysteine.

In certain embodiments, the target compound B is bound to the azepine moiety via an amine —NH— derived from the ε-NH₂ of lysine.

In certain embodiments, the chelating compound is selected from

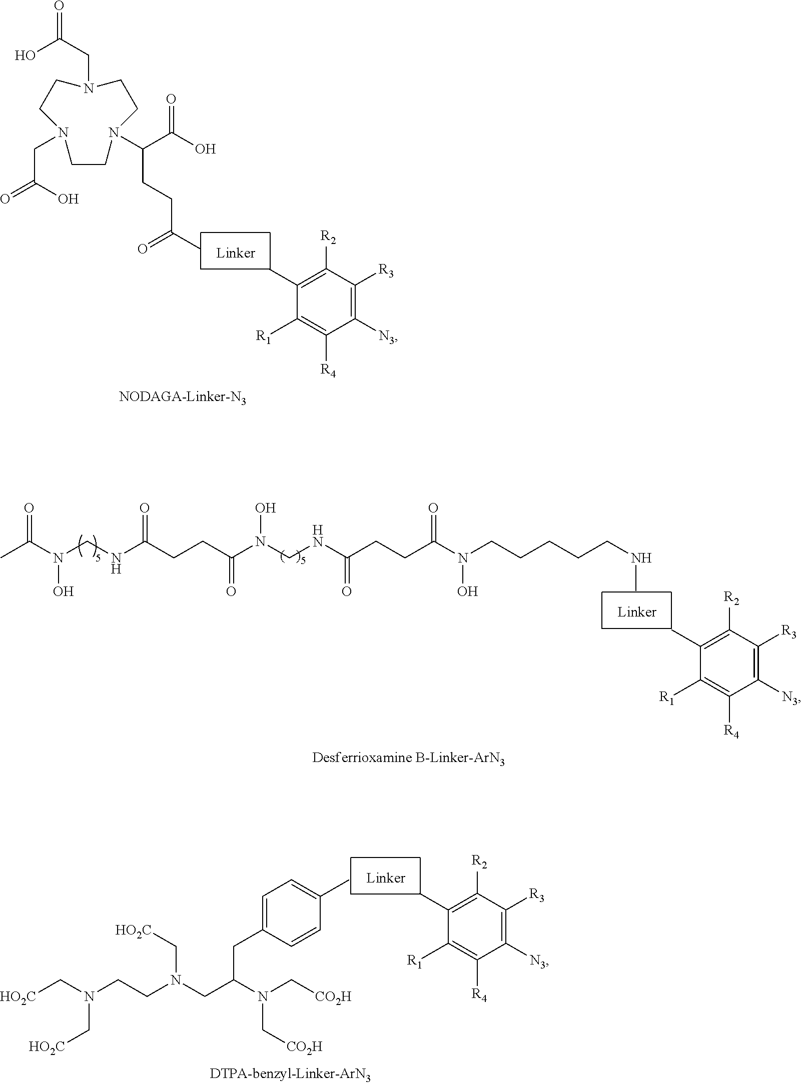

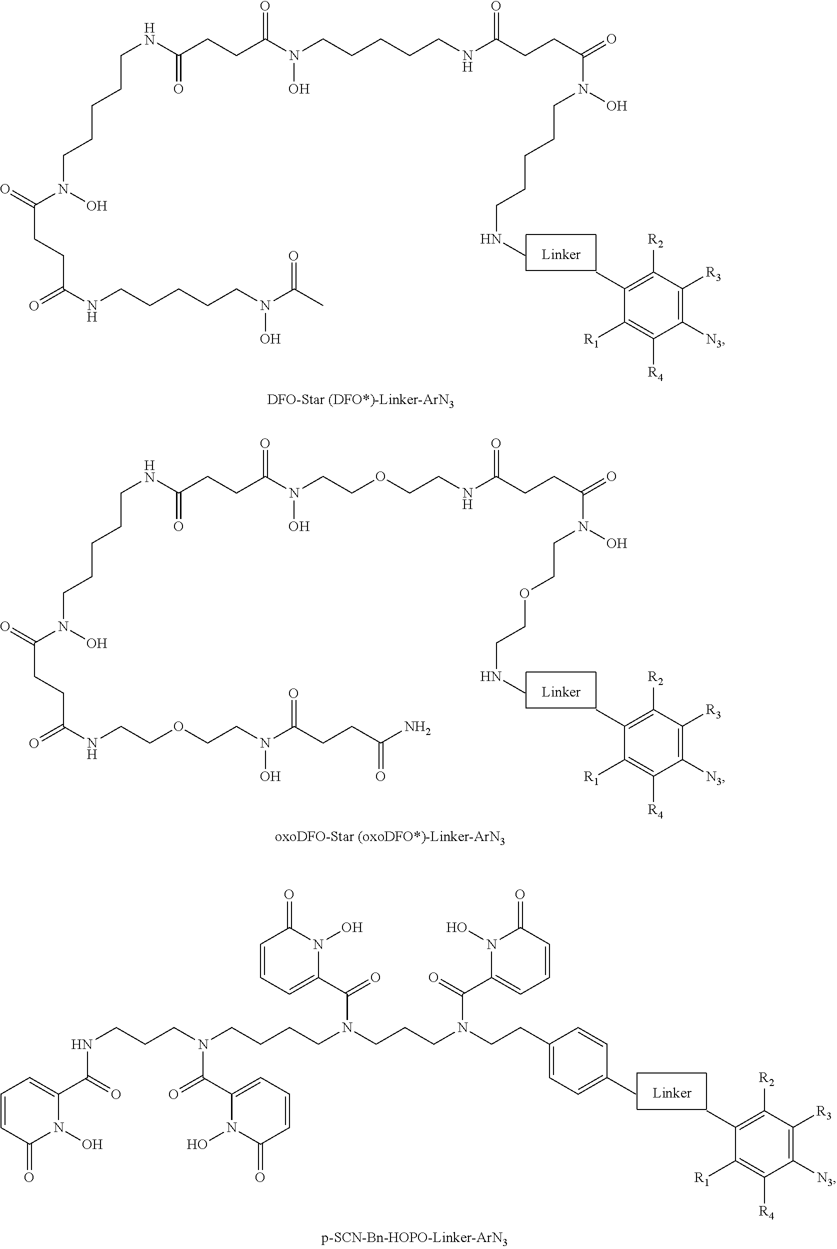

wherein the moiety

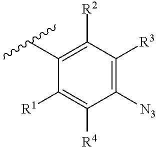

is named “ArN₃” and R¹, R², R³, and R⁴ are defined as R¹ₙ of formula (1).

In certain embodiment, the chelating compound is selected from

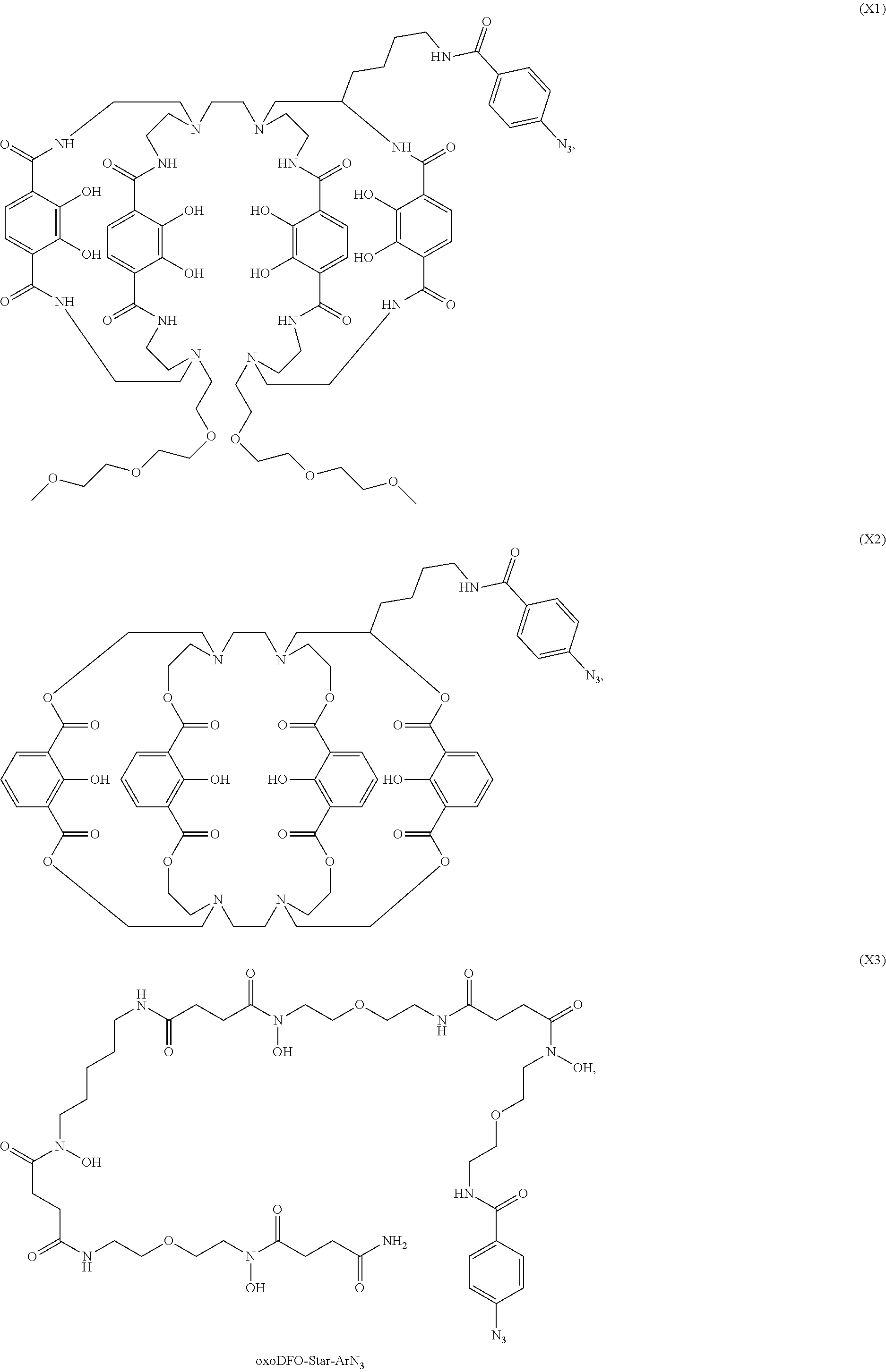 [SMILES: COCCOCCOCCN1CCNC(=O)c2ccc(c(O)c2O)C(=O)NCCN(CCN2CCNC(=O)c3ccc(c(O)c3O)C(=O)NCCN(CCOCCOCCOC)CCNC(=O)c3ccc(c(O)c3O)C(=O)NC(CCCCNC(=O)c3ccc(N=[N+]=[N-])cc3)C2)CCNC(=O)c2ccc(c(O)c2O)C(=O)NCC1 ; [N-]=[N+]=Nc1ccc(C(=O)NCCCCC2CN3CCOC(=O)c4cccc(c4O)C(=O)OCCN(CCOC(=O)c4cccc(c4O)C(=O)O2)CCN2CCOC(=O)c4cccc(c4O)C(=O)OCCN(CCOC(=O)c4cccc(c4O)C(=O)OCC2)CC3)cc1 ; [N-]=[N+]=Nc1ccc(C(=O)NCCOCCN(O)C(=O)CCC(=O)NCCOCCN(O)C(=O)CCC(=O)NCCCCCN(O)C(=O)CCC(=O)NCCOCCN(O)C(=O)CCC(N)=O)cc1]

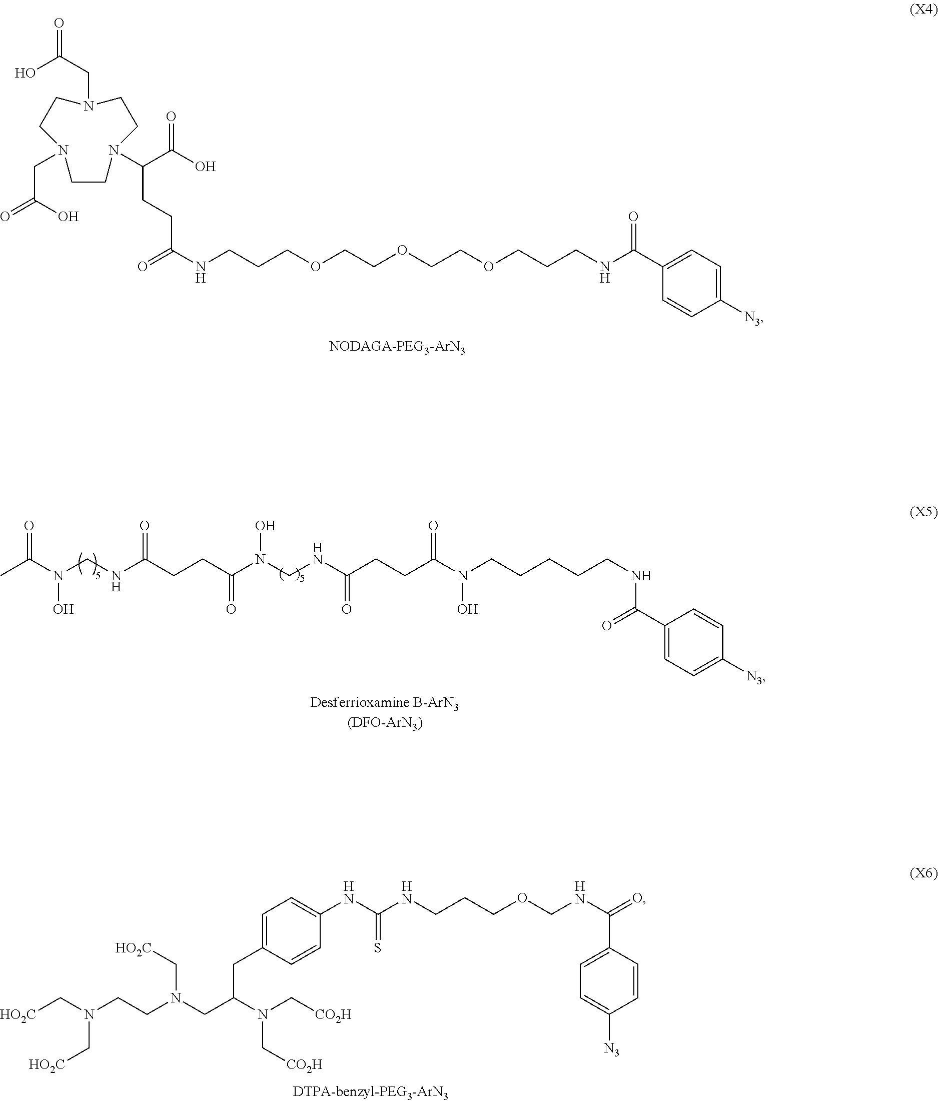

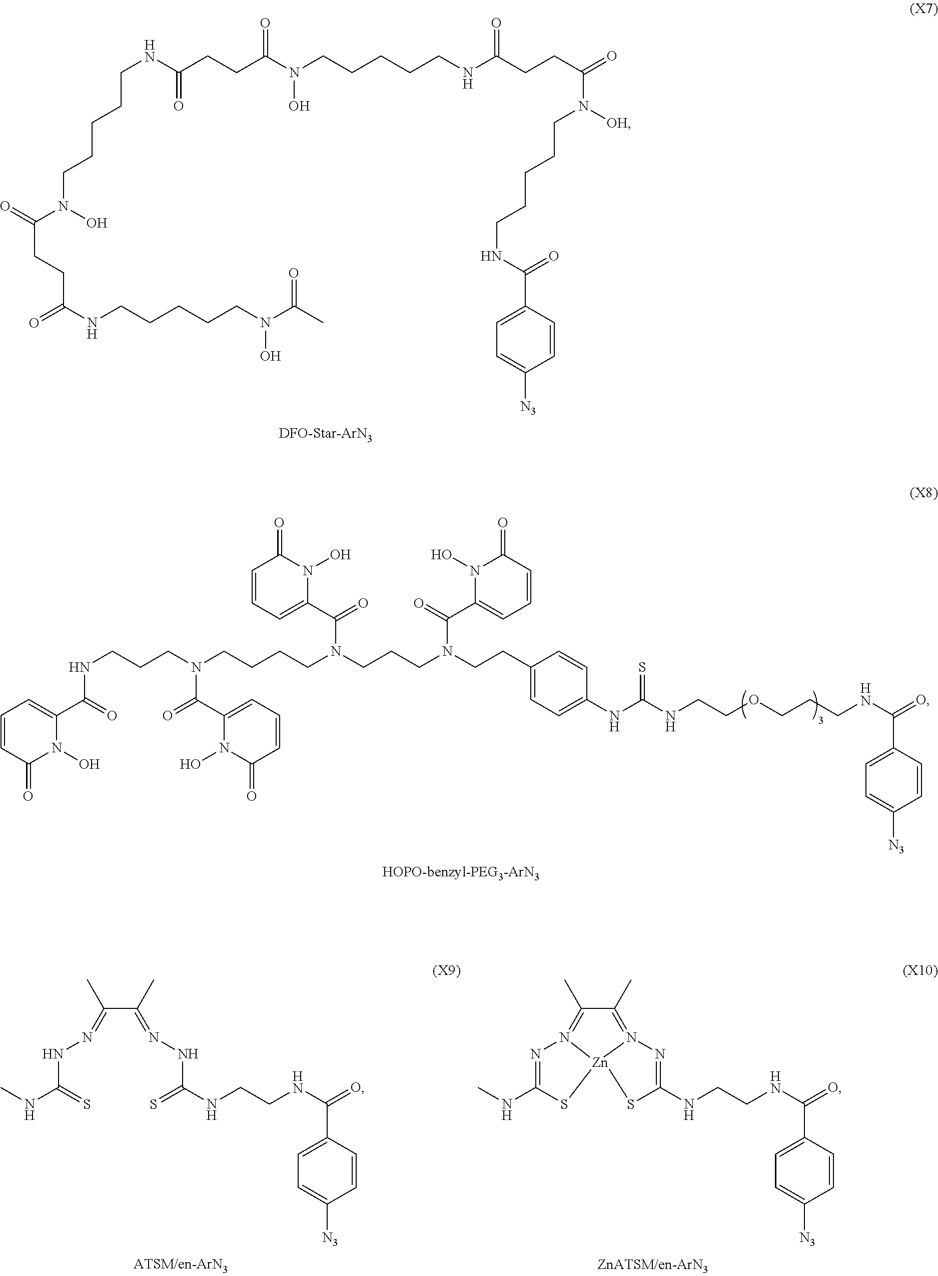

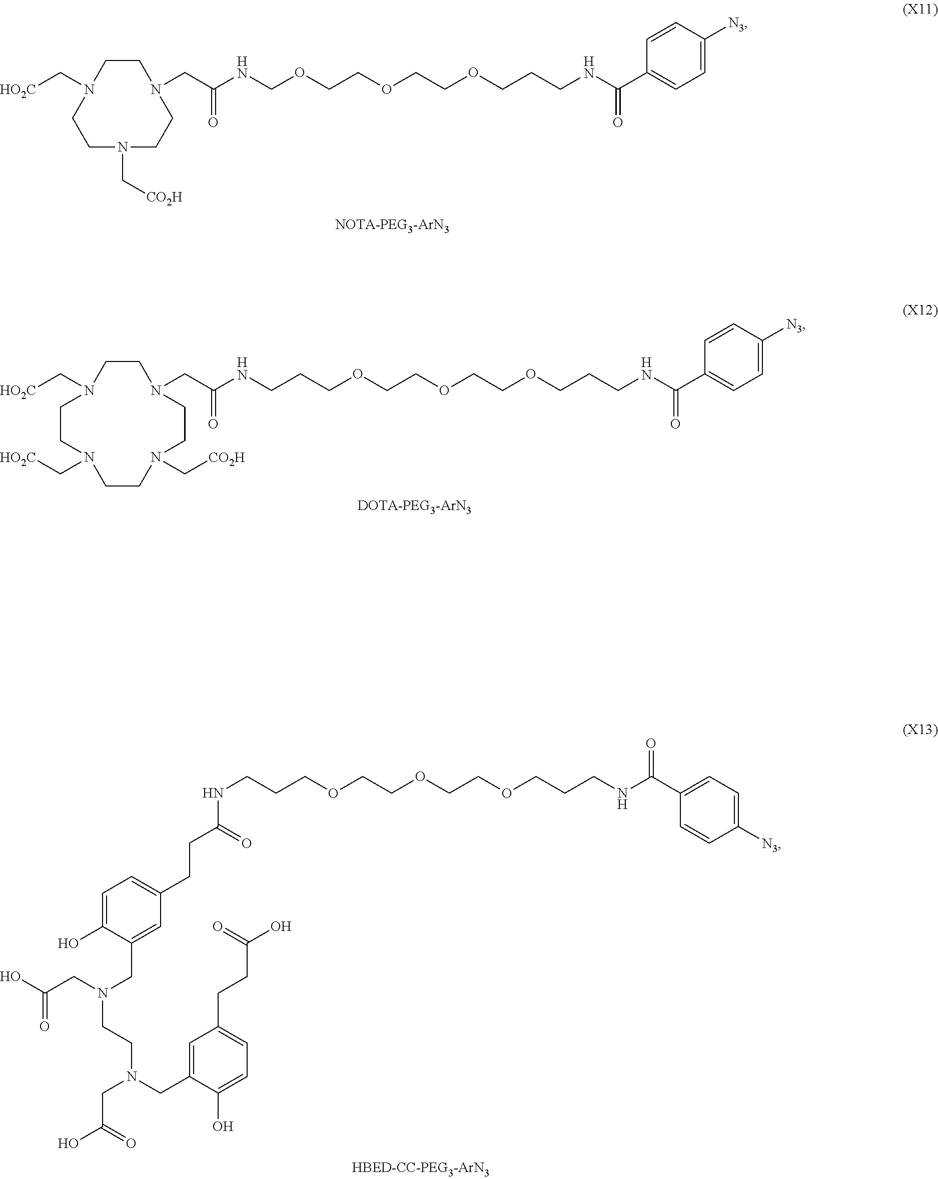 [SMILES: [N-]=[N+]=Nc1ccc(C(=O)NCCCOCCOCCOCNC(=O)CN2CCN(CC(=O)O)CCN(CC(=O)O)CC2)cc1 ; [N-]=[N+]=Nc1ccc(C(=O)NCCCOCCOCCOCCCNC(=O)CN2CCN(CC(=O)O)CCN(CC(=O)O)CCN(CC(=O)O)CC2)cc1 ; [N-]=[N+]=Nc1ccc(C(=O)NCCCOCCOCCOCCCNC(=O)CCc2ccc(O)c(CN(CCN(CC(=O)O)Cc3cc(CCC(=O)O)ccc3O)CC(=O)O)c2)cc1]

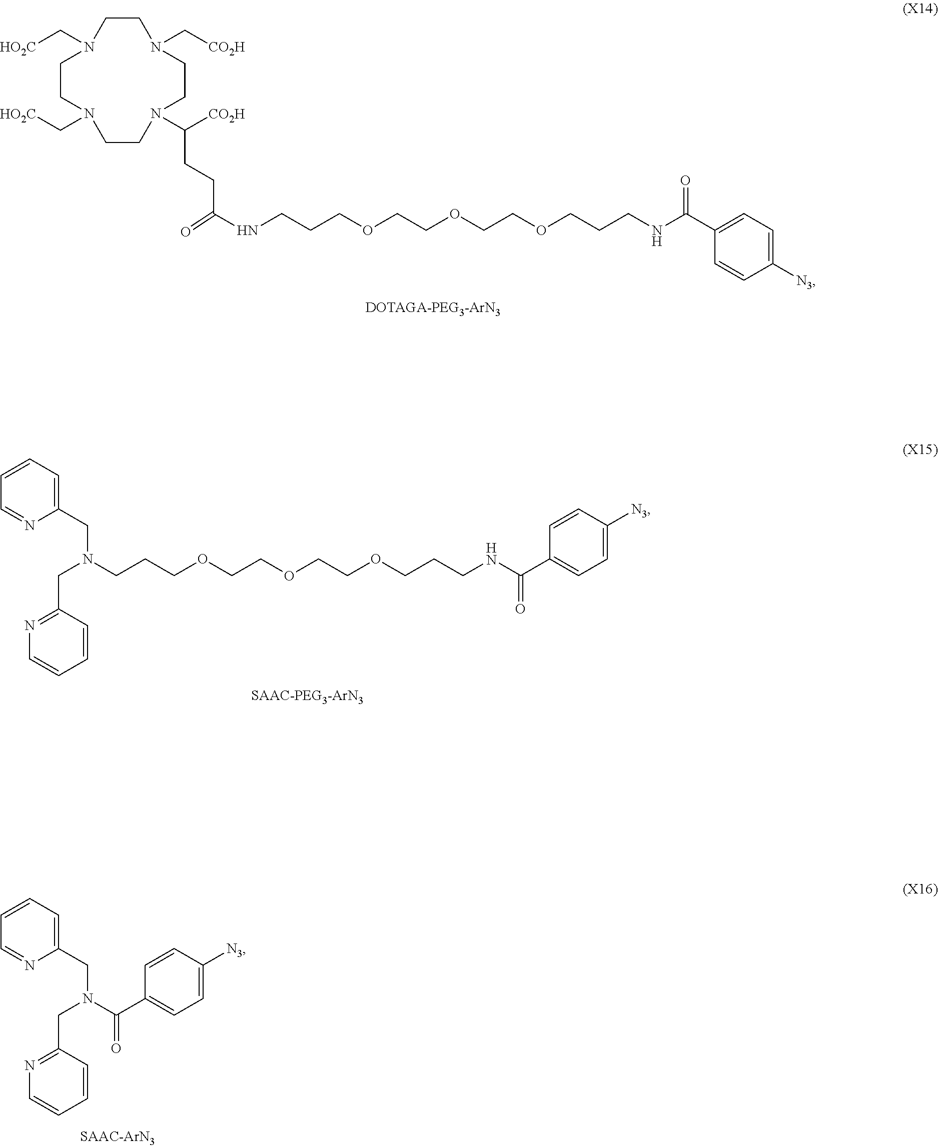 [SMILES: [N-]=[N+]=Nc1ccc(C(=O)NCCCOCCOCCOCCCNC(=O)CCC(C(=O)O)N2CCN(CC(=O)O)CCN(CC(=O)O)CCN(CC(=O)O)CC2)cc1 ; [N-]=[N+]=Nc1ccc(C(=O)NCCCOCCOCCOCCCN(Cc2ccccn2)Cc2ccccn2)cc1 ; [N-]=[N+]=Nc1ccc(C(=O)N(Cc2ccccn2)Cc2ccccn2)cc1]

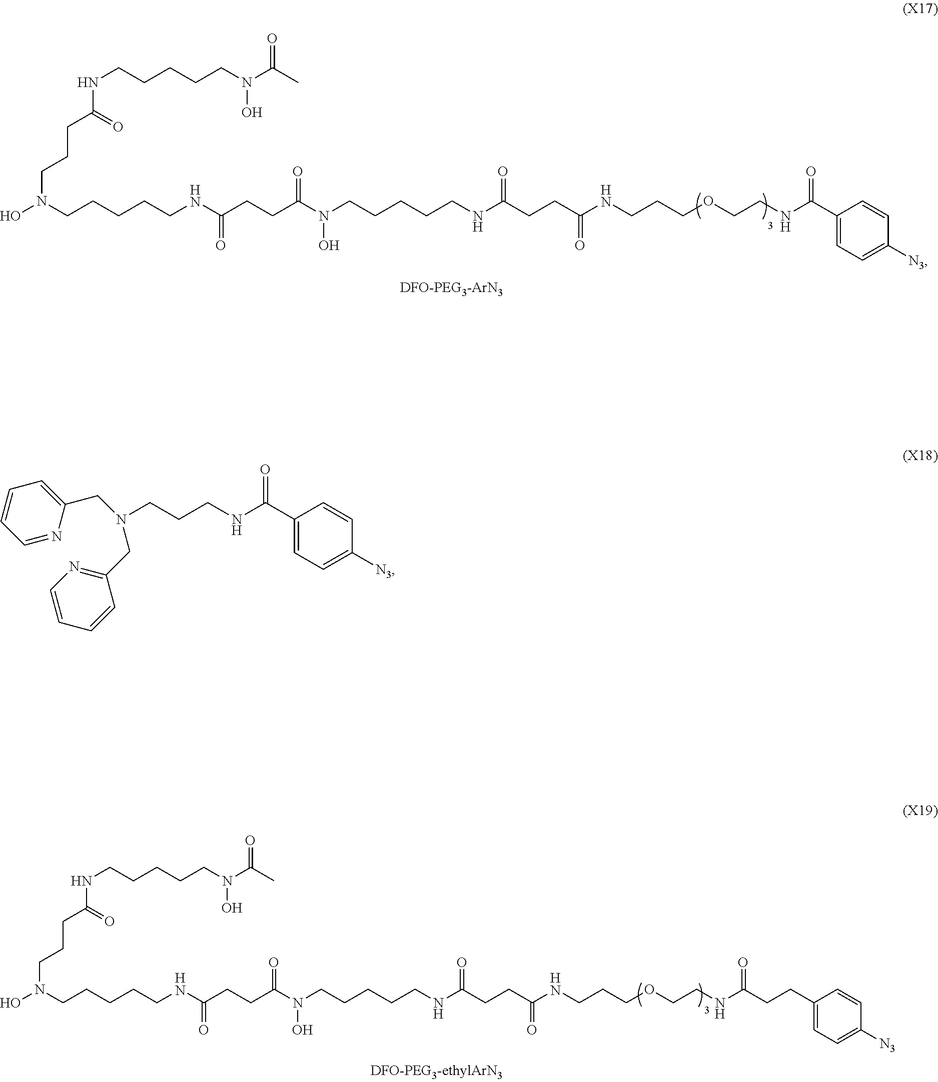

In certain embodiments, the chelating compound is selected from X1, X2, X3, X4, X5, X6, X7, X8, X9, X10, X11, X12, X13, X14, X15 and X16.

In certain embodiments, the chelating compound is selected from X3, X5, X7, X17 and X19.

In certain embodiments, the chelating compound is selected from X3, X5, X7 and X17.

Another aspect of the invention relates to a method for preparing a photoradiolabelled compound in a one-pot reaction comprising separate photoconjugation and radiolabelling steps without a purification step between these steps comprising
ai. providing a reaction mixture comprising at least one chelating compound,
aii. in a radiolabelling step,
   adding a radioactive ion of a radionuclide adjusting the pH to pH<7, in particular pH 3.5 to <7,
aiii. in a photoconjugation step,
   adjusting the pH to pH>7, in particular pH>8, more particularly pH 8 to 11,
   adding a target compound B comprising an amine and/or thiol moiety,
   radiation of the reaction mixture with light at a wavelength selected from 200 nm to 420 nm yielding a photoradiolabelled compound,
   or
bi. providing a reaction mixture comprising
   at least one chelating compound
bii. in a photoconjugation step,
   adjusting the pH to pH>7, in particular pH>8, more particularly pH 8 to 10,
   adding at least one target compound B comprising an amine and/or thiol moiety,
   radiation of the reaction mixture with light at a wavelength selected from 200 nm to 420 nm yielding a photoconjugated intermediate compound,
biii. in a radiolabelling step,
   adding a radioactive ion of a radionuclide
   adjusting the pH to pH<7, in particular pH 3.5 to <7, yielding a photoradiolabelled compound,
wherein the chelating compound is a compound of formula 1,

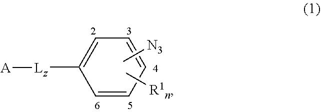

wherein
A is a chelator suitable for coordinating an ion of a radionuclide at acidic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂—CHF₂, —SOCF₃, —SC₂CF₃, —SO₂—NR²R³, —CN, —NO₂, —F, —CI, —Br or —I, in particular ——OH, —OR⁴, —CN, —NO₂, —F, —CI, —Br, or —I, with
   R² and R³ being independently selected from C₁₋₆-alkyl, C₂₋₆-alkenyl and C₂-₆-alkynyl,

R⁴ being selected from C₁₋₆-alkyl, C₂₋₆-alkenyl and C₂₋₆-alkynyl which may optionally be substituted with —F, —CI, —Br or —I.

In certain embodiments, n is 0, 1, 2 or 3, in particular 0 or 1, more particularly 0, and R¹ and —N₃ are positioned in such a way that at least one of the positions 2 to 6 of the phenyl moiety that are next to —N₃ is unsubstituted.

The method according to the invention is directed towards radiolabelling of a chelator moiety of a chelating compound and photoconjugation of an aryl-azide moiety of said chelating compound to a target compound in a one-pot reaction. In the one-pot reaction, either the radiolabelling step can be performed before the photoconjugation step or the photoconjugation step can be performed before the radiolabelling step. There is no need to perform a purification step between the two steps allowing a fast and simple preparation of photoradiolabelled compounds.

The radiolabelling is performed under acidic conditions. At acidic pH, radioactive ions are soluble in an aqueous solution.

In the photoconjugation step, irradiation of the aryl-azide releases N₂ forming a singlet arylnitrene, which at room temperature undergoes extremely fast intramolecular rearrangement to give ketenimines (or benzazirine) intermediates. Ketenimines react relatively slowly with oxygen, protons and water, but undergo rapid nucleophilic addition with amines or thiols of said target compound. The addition is facilitated if the amine, e.g. ε-NH₂ of lysine, or thiol moiety, e.g. -SH of cysteine, is deprotonated. Deprotonation is achieved by adjusting the pH to pH>7 in the photoconjugation step.

In certain embodiments, the target compound comprises a primary, secondary or tertiary amine and/or thiol moiety.

In certain embodiments, the target compound comprises a primary or secondary amine and/or thiol moiety.

In certain embodiments, the target compound comprises a primary or secondary amine —NHR^{h} and/or thiol moiety with R^{h} being a residue that does not react with the chelating compound under the reaction conditions of the method according to the invention.

In certain embodiments, the target compound comprises a primary or secondary amine (—NHR^{h}) and/or thiol moiety (—SH) with R^{h} being H or substituted or unsubstituted C₁₋₁₂-alkyl.

In certain embodiments, the target compound comprises a primary or secondary amine (—NHR^{h}) with R^{h} being H or C₁₋₁₂-alkyl, particularly C₁₋₆-alkyl.

In certain embodiments, the target compound comprises a cysteine and/or lysine.

In certain embodiments, the target compound comprises a lysine.

The radiolabelling step is finished when the radionuclide has been completely complexed by the chelator moiety of the chelating compound. The reaction can be monitored using radioactive chromatography including thin-layer chromatography, HPLC and ion exchange methods.

The photolabelling step is finished when the photo-induced degradation of the arylazide is complete. The time required depends on the light source, the light power, the wavelength and the geometry of the light beam, as well as the geometry of the reaction (shape of the reaction vessel, material used). Usually, the reaction is complete in less than one hour. Under optimized conditions, the reaction is complete in <10 min.

The first step (either radiolabelling or photoconjugation) is finished before the second step (either photoconjugation or radiolabelling) is performed.

In contrast to known labelling methods, the method according to the invention is performed in a one-pot reaction without a purification step. Usually, the method can be performed in less than one hour, particularly in less than 15 min.

For the radiolabelling, at least one ion of a radionuclide is required.

In certain embodiments, the radionuclide is selected from ⁴³Sc, ⁴⁴Sc, ⁴⁷Sc, ⁴⁵Ti, ⁵¹Cr, ⁵¹Mn, ⁵²Mn, ^{52m}Mn, ⁵²Fe, ⁵⁵Co, ⁵⁷Ni, ⁶⁰Cu, ⁶¹Cu, ⁶²Cu, ⁶⁴Cu, ⁶⁷Cu, ⁶⁵Zn, ⁶⁶Ga, ⁶⁷Ga, ⁶⁸Ga, ⁶⁹Ge, ⁷¹As, ⁷²As, ⁷⁴As, ⁷⁶As, ⁷⁷As, ⁸²Rb, ^{82m}Rb, ⁸²Sr, ⁸³Sr, ⁸⁹Sr, ⁸⁶Y, ⁹⁰Y, ⁸⁹Zr, ⁹⁷Zr, ⁹⁰Nb, ^{94m}Tc, ^{99m}Tc, ⁹⁷Ru, ¹⁰⁵Rh, ¹¹¹Ag, ^{110m}In, ¹¹¹In, ^{117m}Sn, ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, ¹⁷⁸Ta, ¹⁸⁶Re, ¹⁸⁸Re, ¹⁹²Ir, ^{195m}Pt, ¹⁹⁸Au, ^{197m}Hg, ²⁰¹Tl, ²¹²Pb, ²¹²Bi, ²¹³Bi, ²¹¹At, ²²³Ra, ²⁵⁵Ac.

In certain embodiments, the radionuclide is selected from ⁴³Sc, ⁴⁴Sc, ⁴⁷Sc, ⁶⁰Cu, ⁶¹Cu, ⁶²Cu, 64Cu, ⁶⁷Cu, ⁶⁷Ga, ⁶⁸Ga, ⁸⁶Y, ⁹⁰Y, ⁸⁹Zr, ^{99m}Tc, ¹¹¹In, ¹⁵³Sm, ¹⁴⁹Tb, ¹⁵²Tb, ¹⁵⁵Tb, ¹⁶¹Tb, ⁷⁷Lu, ¹⁸⁶Re, 188Re, ²¹²Pb, ²¹²Bi, ²¹³Bi, ²²³Ra, ²⁵⁵Ac.

In certain embodiments, the radionuclide is selected from ⁶⁸Ga, ⁸⁹Zr, ⁶⁴Cu, ⁶⁷Cu , ⁹⁰Y, ^{99m}Tc, ¹⁷⁷Lu, ²¹²Pb, ²²⁵Ac.

In certain embodiments, the radionuclide is ⁸⁹Zr.

For the method according to the invention, the radioactive ion has to be soluble under acidic conditions. To enhance the solubility and to stabilize the radioactive ion at acidic pH, suitable co-ligands may be added.

In certain embodiments, a co-ligand is added to the reaction mixture.

In certain embodiments, acetate, oxalate or chloride is added to the reaction mixture.

The method according to the invention can be performed using one type of chelating compound, for example a chelating compound comprising the chelator Desferrioxamine B (DFO), and one type of radioactive ion, e.g. ⁸⁹Zr.

Terms and Definitions

As used herein the term “small molecule” refers to a moiety of a molecular mass of less than 1500 Daltons, in particular a moiety of a molecular mass of less than 1000 Daltons, more particularly a moiety of a molecular mass of less than 500 Daltons.

The term “nanoparticle” relates to particle species of variable chemical composition in the size range of 1 nanometer to 250 nanometers. In particular, nanoparticles made from metal oxides or carbon-based materials, and in particularly nanoparticles made from iron oxides or graphene.

The term derivative in the context of the present invention relates to a compound that is derived from a similar compound (parent compound) by a chemical reaction. The term also includes structural analogues, i.e. compounds that differ from a parent compound in one or more atoms or one or more atom groups.

A C₁-C₆ alkyl in the context of the present specification signifies a saturated linear or branched hydrocarbon having 1, 2, 3, 4, 5 or 6 carbon atoms. Non-limiting examples for a C₆ alkyl include methyl, ethyl, propyl, prop-2-enyl, n-butyl, 2-methylpropyl, tert-butyl, but-3-enyl, prop-2-inyl and but-3-inyl, 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-inyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety. In certain embodiments, a C₁-C₄ alkyl is a methyl, ethyl, propyl or butyl moiety.

The term unsubstituted Cₙ alkyl when used herein in the narrowest sense relates to the moiety if used as a bridge between moieties of the molecule, or —CₙH₂ₙ₊₁ if used in the context of a terminal moiety.

The term Cₙ alkenyl in the context of the present specification signifies a saturated linear or branched hydrocarbon comprising one or more double bonds. An unsubstituted alkenyl consists of C and H only. A substituted alkenyl may comprise substituents as defined herein for substituted alkyl.

The term Cₙ alkynyl in the context of the present specification signifies a saturated linear or branched hydrocarbon comprising one or more triple bonds and may also comprise one or more double bonds in addition to the triple bond(s). An unsubstituted alkynyl consists of C and H only. A substituted alkynyl may comprise substituents as defined herein for substituted alkyl.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense that is covalently linked to an atom that is not carbon or hydrogen, particularly to an atom selected from N, 0, F, B, Si, P, S, Cl, Br and I, which itself may be—if applicable- linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, choride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof.

The term peptide in the context of the present specification relates to a molecule consisting of up to 50 amino acids, in particular 8 to 30 amino acids, more particularly 8 to 15amino acids, that form a linear chain wherein the amino acids are connected by peptide bonds.

In the context of the present specifications the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues.

In the context of the present specification, the term antibody refers to whole antibodies including but not limited to immunoglobulin type G (IgG), type A (IgA), type D (IgD), type E (IgE) or type M (IgM), any antigen binding fragment, e.g. fragment crystallizable (Fc) region, or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (CL). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

The term antibody-like molecule in the context of the present specification refers to a molecule capable of specific binding to another molecule or target with high affinity/a Kd≤10E−8 mol/l. An antibody-like molecule binds to its target similarly to the specific binding of an antibody. The term antibody-like molecule encompasses a repeat protein, such as a designed ankyrin repeat protein (Molecular Partners, Zürich), an engineered antibody mimetic proteins exhibiting highly specific and high-affinity target protein binding (see US2012142611, US2016250341, US2016075767 and US2015368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins.

The term antibody-like molecule further encompasses a polypeptide derived from protein A domains, a polypeptide derived from fibronectin domain FN3, a polypeptide derived from consensus fibronectin domains, a polypeptide derived from lipocalins, a polypeptide derived from Zinc fingers, a polypeptide derived from Src homology domain 2 (SH2), a polypeptide derived from Src homology domain 3 (SH3), a polypeptide derived from PDZ domains, a polypeptide derived from gamma-crystallin, a polypeptide derived from ubiquitin, a polypeptide derived from a cysteine knot polypeptide and a polypeptide derived from a knottin, a polypeptide derived from a cystatin, a polypeptide derived from Sac7d, a triple helix coiled coil (also known as alphabodies), a polypeptide derived from a Kunitz domain of a Kunitz-type protease inhibitor,a polypeptide derived from a carbohydrate binding module 32-2 and a camelid antibody.

The term protein A domains derived polypeptide refers to a molecule that is a derivative of protein A and is capable of specifically binding the Fc region and the Fab region of immunoglobulins.

The term armadillo repeat protein refers to a polypeptide comprising at least one armadillo repeat, wherein an armadillo repeat is characterized by a pair of alpha helices that form a hairpin structure.

The term humanized camelid antibody in the context of the present specification refers to an antibody consisting of only the heavy chain or the variable domain of the heavy chain (VHH domain) and whose amino acid sequence has been modified to increase their similarity to antibodies naturally produced in humans and, thus show a reduced immunogenicity when administered to a human being. A general strategy to humanize camelid antibodies is shown in Vincke et al. “General strategy to humanize a camelid single-domain antibody and identification of a universal humanized nanobody scaffold”, J Biol Chem. 2009 Jan. 30;284(5):3273-3284, and US2011165621A1.

In the context of the present specification, the term fragment crystallizable (Fc) region is used in its meaning known in the art of cell biology and immunology; it refers to a fraction of an antibody comprising two identical heavy chain fragments comprised of a C_{H}2 and a C_{H}3 domain, covalently linked by disulfide bonds.

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷mol/L when binding to its target, but a dissociation constant at least three orders of magnitude higher in its interaction with a molecule having a globally similar chemical composition as the target, but a different three-dimensional structure.

In the context of the present specification, the term dissociation constant (K_{D}) is used in its meaning known in the art of chemistry and physics; it refers to an equilibrium constant that measures the propensity of a complex composed of [mostly two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ A b ] * [ A g ]}{[ A b A g ]}$$

[Ab]: Concentration of Antibody; [Ag]: Concentration of Antigen; [AbAg]: Concentration of Antibodyantigen Complex

In the context of the present specification, the terms off-rate (Koff;[1/sec]) and on-rate (Kon; [1/sec*M]) are used in their meaning known in the art of chemistry and physics; they refer to a rate constant that measures the dissociation (Koff) or association (Kon) of 5 an antibody with its target antigen. Koff and Kon can be experimentally determined using methods well established in the art. A method for determining the Koff and Kon of an antibody employs surface plasmon resonance. This is the principle behind biosensor systems such as the Biacore® or the ProteOn® system. They can also be used to determine the dissociation constant KD by using the following formula:

$${K}_{D} = \frac{[ {K}_{o f f} ]}{[ {K}_{o n} ]}$$

In the context of the present specification, the term humanized antibody is used in its meaning known in the art of cell biology and biochemistry; it refers to an antibody originally produced by immune cells of a non-human species, the protein sequences of which have been modified to increase their similarity to antibody variants produced naturally in humans.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

The term SAAC relates to the chelator

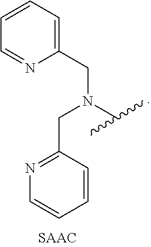 [SMILES: *N(Cc1ccccn1)Cc1ccccn1]

If the term protein is used as a single term such as in a list like “peptide, protein, antibody, an antibody-like molecule, an antibody fragment”, the term “protein” is not to be understood as generic term but as differentiation from the other terms. In this sense, a protein comprises 50 or more amino acids that form a linear chain wherein the amino acids are connected by peptide bonds and the protein is not a peptide (less than 50 amino acids), an antibody, an antibody-like molecule or an antibody fragment. An example for “protein” as single term is human serum albumin.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1. UHPLC chromatograms showing quantitative radiolabelling to give [⁸⁹Zr][ZrDFO-ArN₃]⁺ (blue trace, RCP>99%, RCC>99%) and electronic absorption chromatograms measured at 220 nm (green), 254 nm (red) and 280 nm (black) showing co-elution with an authenticated sample of non-radioactive Zr— DFO-ArN₃.

FIG. 2. Kinetic data on the photochemically induced degradation of compound DFO-ArN₃ during irradiation with UV light (365 nm). (A) Normalised UHPLC chromatograms recorded between 0-25 min. (50% LED power). * indicates starting material (DFO-ArN₃). (B) Kinetic plot showing the change in concentration of DFO-ArN₃versus irradiation time (min.) using different LED intensities. Note, data are fitted with a first-order decay (R²>0.999 for each data set) and the observed first-order rate constants, k_{obs}/min⁻¹ are shown inset. (C) Plot of the normalised rate constant versus the normalised LED intensity confirming that photodegradation is first-order (gradient ˜1.0) with respect to light intensity.

FIG. 3. DFT calculated (uB3LYP/6-311++G(d,p)/PCM) reaction coordinate showing the relative calculated differences in free energy (ΔG/kJ mol⁻¹), enthalpy (ΔH/kJ mol⁻¹) and entropy (ΔS/J K⁻¹ mol⁻¹, at 298.15 K) of the various intermediates and transition states that connect arylazide (PhN₃) with the N-methyl-cis-azepin-2-amine product. Photochemically induced reactivity of arylazides proceeds via the ground state open-shell singlet nitrene (¹A₂ state) corresponding to the (pₓ)¹(p_{y})l electronic configuration where the py orbital on the N atom lies in the plane of the C₆H₅ ring.

FIG. 4. Overlay of the experimentally measured electronic absorption spectrum of DFO-ArN₃ and the TD-DFT (uB3LYP/6-311++G(d,p)/PCM) calculated spectrum of the model compound arylazide (PhN₃). Note that the calculated spectrum was produced by using Lorentzian broadening, 20 nm full-width at half maximum. Calculated energies and oscillator strengths (f/a.u.) of the bands corresponding to transitions to the first six excited singlet states with non-zero expectation values are shown by vertical red lines (band details inset). For reference, band energies to the excited triplet states are shown by vertical green lines. The simulated spectrum and all calculated energies are x-shifted by Δ=+12 nm for clarity.

FIG. 5. DFT calculated (B3LYP/6-311++G(d,p)/PCM) molecular orbital diagram showing electron density isosurfaces of the three highest occupied molecular orbitals (HOMOs) and three lowest unoccupied molecular orbitals (LUMOs) for the model compound arylazide (PhN₃). Note that the isosurfaces were generated by using a contour value of 0.035 and correspond to 96.5% of the total electron density.

FIG. 6. [⁸⁹Zr] ZrDFO-azepin-antibody radiolabelling kinetics and stability data. (A)

Radio-ITLC chromatograms showing the kinetics of formation of [⁸⁹Zr] ZrDFO-azepin-antibody versus time using a pre-functionalised DFO-azepin-antibody sample prepared with an initial chelate-to-monoclonal antibody ratio of 26.4-to-1. (B) Plot of the percentage radiochemical conversion (RCC) versus time using samples of DFO-azepin-antibody pre-conjugated at different initial chelate-to-monoclonal antibodies ratios. (C) Radioactive SEC-UHPLC confirming that [⁸⁹Zr] ZrDFO-azepin-antibody remains stable with respect to change in radiochemical purity during incubation in human serum at 37° C. for 92 h.

FIG. 7. Characterisation data for the simultaneous one-pot photoradiochemical synthesis of [⁸⁹Zr]ZrDFO-azepin-antibody. (A) Radio-iTLC chromatograms showing control reactions in the absence of DFO-ArN₃ (no chelate, green) or monoclonal antibody (yellow), ⁸⁹ZrDFO-ArN₃⁺ before irradiation (purple) and the crude products after irradiation with at 365 nm (black) and 395 nm (red) (B)

Analytical PD-10-SEC elution profiles showing the [⁸⁹Zr][ZrDTPA]⁻ control (green, equivalent to the no chelate control confirming no non-specific binding of ⁸⁹Zr⁴⁺ ions to the monoclonal antibody), a control reaction without monoclonal antibody (yellow), crude reaction mixtures after irradiation and DTPA quenching at 365 nm (black) and 395 nm (red), and the purified product (blue). (C) SEC-UHPLC chromatograms of the crude and purified product.

FIG. 8 Illustration of three mechanistically distinct routes toward radiolabelled antibodies and other proteins.

FIG. 9 Characterisation data for the radiochemical synthesis of [⁶⁸Ga]GaNODAGA-azepin-antibody. (A) Radio-iTLC chromatograms, (B) analytical PD-10-SEC elution profiles, and (C) SEC-UHPLC chromatograms of the crude and purified product.

FIG. 10 Characterisation data for the one-pot photoradiochemical synthesis of [⁶⁸Ga]GaNODAGA-azepin-antibody from pre-purified mAb. (A) RadioiTLC chromatograms, (B) analytical PD-10-SEC elution profiles, and (C) SECUHPLC chromatograms of the crude and purified product.

FIG. 11. Chemical structures of photoactivatable macrocyclic chelates.

FIG. 12. Normalised RP-UHPLC data showing, (green) a single peak for the elution of NOTA-PEG₃-ArN₃ (1), (red) a single peak observed for non-radioactive complex [⁶⁸GaNOTA-PEG₃-ArN₃]⁺ (blue) co-elution of [⁶⁸GaNOTA-PEG₃-ArN₃]⁺ confirming the identity of the radioactive complex, and (black) a single peak formed after irradiation of [⁶⁸GaNOTA-PEG₃-ArN₃]⁺ (365 nm, 15 min.).

FIG. 13. Characterisation data for the one-pot photoradiochemical synthesis of [⁶⁸Ga]GaNOTA-azepin-antibody from pre-purified monoclonal antibody. (A) Radio-iTLC chromatograms, (B) analytical PD-10-SEC elution profiles, and (C) SEC-UHPLC chromatograms of the crude and purified product.

FIG. 14 Chelating compounds DFO-ArN₃ and DFO-PEG₃-ArN₃.

FIG. 15 Radiolabelling of human serum albumin. (A) Radioactive thin-layer chromatography data showing the labelled protein at Rf=0.0, (B) Radioactive analytical size-exclusion chromatography using PD-10 columns showing the labelled and purified protein (black) eluting in the high molecular weight fraction in the first ˜1.8 mL, (C) Electronic absorption size-exclusion high-performance liquid chromatography showing the elution of the protein fraction after radiolabeling for the crude and purified samples.

FIG. 16 Radiolabelling of an scFV-Fc protein. (A) Radioactive thin-layer chromatography data showing the labelled protein at Rf=0.0, (B) Radioactive analytical size-exclusion chromatography using PD-10 columns showing the labelled and purified protein (black) eluting in the high molecular weight fraction in the first ˜1.8 mL, (C) Electronic absorption size-exclusion high-performance liquid chromatography showing the elution of the protein fraction after radiolabeling for the crude and purified samples.

FIG. 17 Radiolabelling of a human IgG1 protein. (A) Radioactive thin-layer chromatography data showing the labelled protein at Rf=0.0, (B) Radioactive analytical size-exclusion chromatography using PD-10 columns showing the labelled and purified protein (black) eluting in the high molecular weight fraction in the first ˜1.8 mL, (C) Electronic absorption size-exclusion high-performance liquid chromatography showing the elution of the protein fraction after radiolabeling for the crude and purified samples.

FIG. 18 shows an example for a photoradiolabelled compound: ^{99m}Tc labelled SAAC chelate bound to an antibody fragment via an azepine moiety and a linker, the azepine moiety was formed upon reaction between a lysine of the antibody fragment and the aryl-N₃ moiety of the chelating compound.

FIG. 19 shows radiolabeling of the SAAC-ArN₃ chelate. (A) Reaction scheme, (B) radioactive HPLC data showing the different radioactive small molecule species formed during initial radiolabelling of the ASAAC-ArN₃ chelate with ^{99m}Tc, (C) radioactive HPLC data showing the improved radiolabeling of ^{99m}TcSAAC-ArN₃ after some optimization work.

FIG. 20 shows the rate of change in the relative concentrations of the different species versus time during irradiation with a 365 nm LED lamp. (A) different species shown in colour code, (B) a standard reaction vial and (C) a cuvette that allows more efficient transmission of the light into the sample solution.

FIG. 21 shows size-exclusion PD-10 data. (A) Reaction scheme for labelling a scFv-Fc protein, (B) analysis of the crude reaction mixture after labelling a scFv-Fc protein, and (C) the equivalent profile after purification of the radiolabelled protein fraction from small molecule contaminants.

FIG. 22 shows (A) the stability of the radiolabelled protein when challenged with 0.2 M histidine measured by size-exclusion analytical PD10 analysis (27% loss of radiotracer after 9h), and (B) challenged with human serum albumin measured by size-exclusion HPLC (no significant change after 20h).

### EXAMPLES

The method according to the invention is directed towards simultaneous radiolabelling of a chelator moiety of a chelating compound and photoconjugation of an aryl-azide moiety of said chelating compound to a target compound. Irradiation of the aryl-azide releases N₂ forming a singlet arylnitrene, which at room temperature undergoes extremely fast intramolecular rearrangement to give ketenimines (or benzazirine) intermediates. Ketenimines react relatively slowly with oxygen, protons and water, but undergo rapid nucleophilic addition with amines or thiols of the target compound B. The addition is facilitated if the amine or thiol moiety is deprotonated.

For instance, suitable target compounds are various peptides and proteins that comprise an amine or a thiol moiety e.g. in the side chain of amino acids such as lysine or cysteine. Suitable full-length antibodies may be selected from trastuzumab, cetuximab, bevacizumab, panitumumab, ibritumomab tiuxetan, J591, fresolimumab, rituximab, brentuximab, lumretuzumab, U36, R1507, ranibizumab, DN30, 7E11, particularly trastuzumab. A suitable antibody fragment is onartuzumab. Suitable proteins may be selected from albumin, transferrin, ceruloprotein, globulins (in general), fibrinogen and other proteins circulating in the blood pool, particularly serum albumin.

The invention is further demonstrated by the examples described herein showing photoradiolabelling using a full-length antibody, an antibody fragment and the protein albumin.

### Example 1: Simultaneous Photoradiolabelling Using DFO-PEG₃-ArN₃

The chelating compound DFO-PEG₃-ArN₃ (FIG. 14) was simultaneously photoradiolabeled using ⁸⁹Zr and human serum albumin (FIG. 15), an antibody fragment (FIG. 16) or a full-length antibody (FIG. 17). DFO-PEG₃-ArN₃ and DFO-PEG³-ethylArN₃ are much more water soluble than DFO-ArN₃ compound. This means that they are a lot easier to work with for radiolabelling proteins with ⁸⁹Zr, both of which are obtained in aqueous solutions. Furthermore, higher radiochemical yields in the region of 75-80% were achieved.

### Example 2: Simultaneous Photoradiolabelling of Antibodies

In proof-of-concept work, it was demonstrated that the photoradiochemical approach showed equivalent successful when radiolabelling either pre-purified fractions of monoclonal antibodies, or starting from fully formulated samples. Reactions were established in which [⁸⁹Zr][Zr(C₂O₄]⁴⁻, and a monoclonal antibody (at an initial chelate-to-monoclonal antibody ratio of ˜29-to-1) were mixed in water and the pH adjusted to ˜8-9. Control reactions were also performed in the absence of either the chelate or the monoclonal antibody. Reactions were then stirred and irradiated using the LED source (365 nm or 395 nm) at room temperature for 10 min.

After irradiation, the mixtures were quenched by the addition of DTPA. Aliquots of the crude mixtures were retained and a fraction was purified by SEC-methods. Crude and purified samples were then analysed by using radio-iTLC, analytical PD-10-SEC and SEC-UHPLC methods (FIG. 7 and Table S3 below). Control reactions confirmed that the ⁸⁹Zr radioactivity specifically bound to the monoclonal antibody (FIG. 7A and 7B, green and yellow traces). Analysis of the crude reaction mixtures also indicated that ˜72-73% (by analytical PD-10-SEC), and ˜67-88% (by SEC-UHPLC) of the ⁸⁹Zr radioactivity was associated with the monoclonal antibody. After purification, the formulated sample of [⁸⁹Zr] ZrDFO-azepin-antibody produced from simultaneous photoradiolabelling using irradiation at 365 nm was isolated with a decay-corrected RCY of 76%, a RCP ˜97% (by SEC-UH PLC) and a molar activity of 0.41 MBq nmol⁻¹ of protein. Interestingly, both the 365 nm and 395 nm LED sources gave equivalent radiochemical conversion. The reaction was complete in <10 min and the entire process, from non-labelled antibody to formulated [⁸⁹Zr] ZrDFO-azepin-antibody was accomplished in <15 min. With a higher intensity light source, it is conceivable that the photoradiochemical synthesis could be accomplished in a few seconds, which would mean that process times are limited only by the purification step.

Comparison of the final RCYs measured between the two-step process and the simultaneous one-pot (one-step) process indicate that the photochemical conjugation efficiency increases from about 3.5% to >75%. This is a remarkable result that means that the chemical efficiency of simultaneous photoradiolabelling is comparable to some of the most efficient thermally mediated conjugation processes (typically ˜60-80%). Under the conditions employed, it is likely that the kinetics of metal ion complexation are similar to the photochemical conjugation step. If ⁸⁹Zr⁴⁺ ions are coordinated first by the DFO-ArN₃ chelate, this limits the possibility of intramolecular reaction between the nucleophilic hydroxamate groups and the photo-generated intermediates. Such an elegant photoradiochemical process is also amenable to full automation which has potential to change the way in which radiolabelled monoclonal antibodies are produced in the clinic.

### Example 3: Two-Step Photochemical Conjugation and ⁸⁹Zr-Radiolabelling of a Monoclonal Antibody

Prior to investigating a simultaneous one-pot photoradiochemical process, experiments were performed using the traditional two-step approach involving an initial photochemical conjugation between DFO-ArN₃ and a monoclonal antibody, followed by ⁸⁹Zr-radiolabelling.

The photochemical conjugation between DFO-ArN₃ and the monoclonal antibody was performed at room temperature for 35 min. using a Rayonet reactor. The DFO-azepin-antibody conjugate was purified by using a combination of size-exclusion chromatography (SEC) methods including spin-column centrifugation and preparative PD-10 gel filtration. Then aliquots of DFO-azepin-antibody were radiolabelled with ⁸⁹Zr using standard conditions.^{[31, 33-35]} Aliquots of the crude radiolabelling mixture were retained and the radiolabelled fraction of [⁸⁹Zr]ZrDFO-azepin-antibody was purified and formulated in sterile PBS by standard SEC methods. Analytical measurements on the crude and purified samples of [⁸⁹Zr]ZrDFO-azepin-antibody were performed using radioactive instant thin-layer chromatography (radio-iTLC), analytical PD-10-SEC and radioactive SEC-UHPLC.

Experiments confirmed that the DFO-azepin-antibody was sample radiolabelled efficiently with ⁸⁹Zr giving with a crude radiochemical conversion (RCC) of >98% after incubating the mixture at room temperature for 15 min. On scaling-up the radiolabelling reaction for use in subsequent cellular and animal experimentations, the final radiochemical yield (RCY) of the purified sample was >99% and the radiochemical purity (RCP) was measured at >99.5% (by analytical PD-10-SEC) and >98% (by SEC-UHPLC).

Additional ⁸⁹Zr-radiolabelling experiments were performed to measure the radiolabelling kinetics and overall RCC of DFO-azepin-antibody samples that were prepared using different initial chelate-to-monoclonal antibody ratios in the photochemical conjugation step (FIG. 7). For each sample, the radiolabelling kinetics was monitored by radio-iTLC (FIG. 7A) and the RCC (%) versus time was plotted (FIG. 7B). These experiments showed linear relation between the initial chelate-to-monoclonal antibody ratio and the overall RCC at saturation (time points >60 min.). Using these data, and also the experimentally measured molar activity of the stock solution of [⁸⁹Zr][Zr(C₂O₄)₄]⁴⁻ (determined by titration with DFO)^{[36]} the conjugation efficiency between DFO-ArN₃ and antibody was estimated to be 3.5±0.4%. Therefore, an initial chelate-to-monoclonal antibody ratio of 26.4 yielded ˜0.85 accessible chelates per monoclonal antibody in the final product.

The radiochemical stability of [⁸⁹Zr]ZrDFO-azepin-antibody with respect to change in the RCP during incubation in human serum at 37° C. for up to 92 h was determined by SEC-UHPLC (FIG. 7C). Experiments confirmed that the ⁸⁹Zr activity remained bound to the monoclonal antibody (<2% decrease in RCP after 92 h) with essentially no trans-chelation serum proteins (transferrin, albumin etc).

### Example 4: One-Pot Pre-Radiolabelling Followed by Photoconjugation Using Two Different pH

The photoradiochemical reaction was tested further by using a one-pot approach. Compound 5 (NODAGA-PEG₃-ArN₃) was pre-radiolabelled with ⁶⁸Ga, re-buffered to pH 8.0, and then prepurified monoclonal antibody was added and the mixture irradiated (FIG. 10). Analytical methods confirmed that one-pot preradiolabelling approach could be completed in <15 min. total time (radiolabelling, photochemical conjugation and purification) to give [⁶⁸Ga]GaNODAGA-azepin-antiboldy in sterile PBS with a decay corrected RCY of 33.9±0.7% (n=3). Development of this fast and efficient one-pot route simplifies the production of radiolabelled proteins by removing the need to synthesise under GMP conditions, then isolate and store the pre-functionalised intermediate. In addition, the one-pot route is suitable for automation using modular radiosynthesis units.

Finally, with a view to expanding the utility of photoradiochemistry, the one-pot approach was tested using a preparation of an antibody. Clinical preparations of an antibody are typically stabilised by the addition of salts, amino acids, anti-oxidants and surfactants. The preparation used herein contains histidine, a,a-trehalose dehydrate and polysorbate 20.

Traditional coupling methods do not tolerate such additives which necessitates pre-purification of the mAb component (usually from a GMP source). Removing the stabilisers risks damaging the protein, and isolation/storage of an intermediate species raises other concerns regarding the long-term biological integrity of the radiolabelling precursor with respect to the parent compound. Methods that allow direct radiolabelling of the formulated

GMPgrade mAbs could potentially redefine the way in which radiopharmaceuticals are prepared for immuno-PET and RIT. Experiments showed that the photochemical approach using the antibody preparation produced [⁶⁸Ga]GaNODAGA-azepin-antibody in a decay-corrected RCY of 23.3±3.4% (n=3). Interestingly, the presence of histidine only slightly reduced the RCY.

From a pharmacokinetic standpoint, the combination of ⁶⁸Ga with long-circulating, full-length mAbs is sub-optimal but this radionuclide is useful for radiolabelling lower molecular weight species like immunoglobulin fragments and peptides. Compound 5 can also be used for complexation of other radionuclides including ⁶⁴Cu. Based on these proof-of-concept studies, the approach was expanded by synthesising a range of compounds for radiolabelling with ⁶⁴Cu, ⁸⁹Zr, ⁹⁰Y, ¹¹¹In, ¹⁷⁷Lu, ²²⁵Ac and others.

One-pot photochemical conjugation and radiolabelling of a monoclonal antibody One-pot photochemical conjugation and radiolabelling reactions were performed in accordance with the following general procedure. To a solution of NODAGA-PEG₃-ArN₃ (5) (160 μg, 2.21×10⁻⁷ mol, 2.21 mM) buffered with NaOAc (0.24 M, pH4.4) was added [⁶⁸Ga][Ga(H₂O)₆]Cl₃(aq.) stock solution (31.8±2.0 MBq, generator 2, n=3) resulting in a total reaction volume of 100 μL. Note, one-pot reactions were not stirred because stirring was found to have no effect on the radiolabelling or the photochemical conjugation efficiency. Reactions were monitored by radio-iTLC. Formation of [⁶⁸Ga]GaNODAGA-PEG₃-ArN₃, ⁶⁸Ga-5, was complete after <5 min. incubation at 23° C. with radiochemical conversion (RCC) >99% (n=3, R_{f}=0.06−0.17 on iTLC, FIG. 10). The pH of the reaction mixture was then adjusted to ˜8.0 by the addition of an aqueous solution of NaHCO₃ (1.0 M, 30 μL added). After adjusting the pH, an aliquot of pre-purified monoclonal antibody (1.134 mg, 7.82'10⁻⁸ mol, reaction concentration=7.875 mg/mL) was added to give an initial chelate-to-antibody ratio of ˜28-to-1 at the start of the photochemical conjugation step (total reaction volume ˜144 μL). The reaction mixture was then irradiated using the LED (100% intensity, 365 nm) for 10 min. at room temperature without stirring. After irradiation, the reaction was quenched by the addition of EDTA (disodium form, 100 μL, 50 mM stock solution, pH7.1, containing 5×10⁻⁶ mol EDTA, 22.6-fold excess with respect to the initial concentration of compound 5; final reaction volume ˜244 μL). Note, the pH of the reaction mixture did not change after addition of the EDTA solution. Aliquots of this crude, quenched reaction mixture were then analysed by using radio-iTLC, PD-10-SEC and SEC-UHPLC analysis.

Radio-iTLC analyses of the crude reactions after irradiation and quenching showed that ˜40% (n=3) of the radioactivity was bound to the antibody (R_{f}=0.0). Note: integration of these radio-iTLC data is unreliable because the radiolabelled antibody fraction partially overlaps with the peak associated with ⁶⁸Ga-5 and the photodegraded ⁶⁸Ga-5 species (R_{f}=0.06-0.17). Nevertheless, analytical PD-10-SEC measurements on the crude reaction mixtures confirmed this observation with an estimated RCP of 38.0±2.0% (n=3). Equivalent decay corrected SEC-UHPLC measurements indicated that the radiolabelled fraction of [⁶⁸Ga]GaNODAGA-azepin-antibody in the crude mixture was 22.0±3.5% (n=3).

Crude reaction mixtures were then purified by preparative PD-10-SEC eluting with PBS (collecting only the high purity 0.0-1.6 mL fraction). Prior to analysis, samples were concentrated using an Amicon Ultra-4 mL centrifugal filter (Millipore, 30 kDa MWCO, 4000 RPM, ˜10 min.). The purified and formulated [⁶⁸Ga]GaNODAGA-azepin-antibody products (pH7.4) were obtained in <15 min. with decay corrected radiochemical yields (RCY) of 33.9±0.7% (n=3). The estimated lower limit on the molar activity (Aₘ/[MBq/nmol] of protein) of the formulated [⁶⁸Ga]GaNODAGA-azepin-antibody samples was 1.02±0.07 MBq/nmol (n=3). Purified products were then reanalysed by radio-iTLC, analytical PD-10-SEC and SEC-UHPLC. The RCP of purified [⁶⁸Ga]GaNODAGA-azepin-antibody was >99% (n=3) by radio-iTLC, 97.6±0.9% (n=3) by analytical PD-10-SEC, and 91.0 ±2.7% (n =3) by SEC-UHPLC.

Appropriate control reactions were also performed. In the absence of the NODAGA-PEG₃-ArN₃ chelate (5) no ⁶⁸Ga-radioactivity bound to the monoclonal antibody in the crude reaction mixtures after irradiation and quenching with EDTA (FIG. S40C, UV/vis trace [green] showing the antibody absorbance at 280 nm, and radioactive trace [red] showing the elution of [⁶⁸Ga][Ga(EDTA)]⁻ on the SEC-UHPLC which was formed after quenching the reaction. PD-10-SEC analysis confirmed that in the control reaction, <1.3% of the activity was present in the 0.0-2.0 mL high molecular weight fraction. Additional control reactions confirmed that no radioactivity associated with the antibody fraction after incubation of [⁶⁸Ga]GaNODAGA-PEG₃-ArN₃ (⁶⁸Ga-5) with the antibody for 10 min. at room temperature in the dark (no irradiation, data not shown—see radiolabelling of formulated antibody preparation, vide infra).

One-Pot Photochemical Conjugation and Radiolabelling of a Preparation of a Monoclonal Antibody

From a mechanistic perspective, the presence of an amino acid (histidine) in the formulation limits the possibilities for conjugation of the antibody with a chelate in situ. The amine group of the amino acid competes with the ε-NH₂ side-chain of accessible lysine residues on the protein in most standard conjugation chemistries. Therefore, radiolabelling antibodies typically requires a pre-purification step to isolate the antibody fraction from other components of the standard formulation.

For instance, a standard antibody preparation as used herein contains L-histidine hydrochloride (9.9 mg), L-histidine (6.4 mg), α,α-trehalose dihydrate (400 mg, α-D-glucopyranosyl-α-D-glucopyranoside), and polysorbate 20 (1.8 mg). After reconstitution with 20 mL of the supplied bacteriostatic water for injection (BWFI), containing 1.1% benzyl alcohol as a preservative, the injectate contains monoclonal antibody at 21 mg/mL, at pH ˜6.0. Thus, the formulation contains a total of 9.31×10⁻⁶ mol of histidine and 2.93×10⁻⁶ mol of antibody (assuming a molecule weight of about 150,000 Da). Therefore, the mole ratio of primary amine groups from histidine to total moles of mAb is approximately 31.7-to-1. The monoclonal antibody has approximately 90 lysine residues. Assuming that mAb_lysine groups are chemically accessible, the histidine-to-mAb_lysine ratio is approximately 0.156 (i.e. one histidine-NH₂ group to 6.4 mAb_lysine groups). Hence, it should be possible to radiolabel the monoclonal antibody directly in the preparation without the need for a pre-purification step. The caveat is that the thermodynamics and kinetics of coupling to histidine-NH₂ are potentially different to that of the antibody-lysine residues. Nevertheless, to test the hypothesis, one-pot photochemical conjugation and radiolabelling experiments were performed using non-purified preparation reconstituted in 18.2 MΩ·cm water.

To a solution of NODAGA-PEG₃-ArN₃ (5) (160 μg, 2.21×10⁻⁷ mol, 2.21 mM) buffered with NaOAc (0.3 M, pH4.4) was added [⁶⁸Ga][Ga(H₂O)₆]Cl₃(aq.) stock solution (35.1±0.5 MBq, generator 2, n=4) resulting in a total reaction volume of 100 μL. Reactions were monitored by radio-iTLC. Formation of [⁶⁸Ga]GaNODAGA-PEG₃-ArN₃, ⁶⁸Ga-5, was complete after 5 min. incubation at 23° C. with radiochemical conversion (RCC) >99% (n=4, R_{f}=0.06−0.17 on radio-iTLC). The pH of the reaction mixture was then adjusted to ˜8.0 by the addition of an aqueous solution of NaHCO₃ (1.0 M, 40 μL added). After adjusting the pH, an aliquot of the preparation was added to the reaction (stock solution prepared by reconstituting 4.5 mg preparation in water (60 μL): reactions contained 28 μL of stock solution which was equivalent to 1.077 mg monoclonal antibody, 7.42×10⁻⁹ mol, final reaction concentration=6.41 mg/mL). The initial chelate-to-antibody ratio of was 29.8 at the start of the photochemical conjugation step (total reaction volume ˜168 μL). The reaction mixture was then irradiated using the LED (100% intensity, 365 nm) for 10 min. at room temperature without stirring. After irradiation, the reaction was quenched by the addition of EDTA (disodium form, 100 μL, 50 mM stock solution, pH 7.1, containing 5×10⁻⁶ mol EDTA, 22.6-fold excess with respect to the initial concentration of compound 5; final reaction volume ˜268 μL). Note, the pH of the reaction mixture did not change after addition of the EDTA solution. Aliquots of this crude, quenched reaction mixture were then analysed by using radio-iTLC and SEC-UHPLC analysis (Figure S40).

Radio-iTLC analyses of the crude reactions after irradiation and quenching showed that 37.3 ±2.4% (n=3) of the radioactivity was bound to the antibody (R_{f}=0.0). Note: integration of these radio-iTLC data is unreliable because the radiolabelled antibody fraction partially overlaps with the peak associated with ⁶⁸Ga-5 and the photodegraded ⁶⁸Ga-5 species (R_{f}=0.06−0.17). Equivalent decay corrected SEC-UHPLC measurements indicated that the radiolabelled fraction of [⁶⁸Ga]GaNODAGA-azepin-antibody in the crude mixture was 10.8±1.9% (n=3).

Crude reaction mixtures were then purified by preparative PD-10-SEC eluting with PBS (collecting only the high purity 0.0-1.6 mL fraction). Prior to analysis, samples were concentrated using an Amicon Ultra-4 mL centrifugal filter (Millipore, 30 kDa MWCO, 4000 RPM, 10 min.). The purified and formulated [⁶⁸Ga]GaNODAGA-azepin-antibody products (pH7.4) were obtained in <15 min. with decay corrected radiochemical yields (RCY) of 23.3±3.4% (n=3). The estimated lower limit on the molar activity (Aₘ/[MBq/nmol] of protein) of the formulated [⁶⁸Ga]GaNODAGA-azepin-antibody samples was 0.92±0.15 MBq/nmol (n=3). Purified products were then reanalysed by radio-iTLC and SEC-UHPLC (Figure S40). The RCP of purified [⁶⁸Ga]GaNODAGA-azepin-antibody was >99% (n=3) by radio-iTLC, and 86.0±2.6% (n=3) by SEC-UHPLC. Lower RCP was observed in SEC-UHPLC analyses of these radiolabelling reactions starting from the antibody preparation because the capacity of the PD-10 columns for preparative purification was insufficient to allow complete purification of the radiolabelled mAb component from the large amount of radiolabelled histidine. In future optimisation work, this issue can be readily resolved by using higher capacity purification methods.

Appropriate control reactions were also performed. After radiosynthesis of [⁶⁸Ga]GaNODAGA-PEG₃-ArN₃ (⁶⁸Ga-5) and re-buffering, an aliquot of the antibody preparation was added to the reaction vessel and the mixture was incubated in the dark at room temperature for ˜10 min. After quenching, radio-iTLC and SEC-UH PLC analysis showed that no ⁶⁸Ga-radioactivity was bound to the monoclonal antibody. In the absence of light, all radioactivity in solution remained as unreacted ⁶⁸Ga-5.

### Example 5: Photochemical Conjugation and ⁶⁸Ga-Radiolabelling of a Monoclonal Antibody Using NOTA-, DOTA- and DOTAGA-PEG₄-ArN₃ (Different pH for Conjugation and Radiolabelling)

Synthesis of the Photoactive Chelates

The photoactive chelates, NOTA-PEG₃-ArN₃ (1), DOTA-PEG₄-ArN₃ (3) and DOTAGA-PEG₄-ArN₃ (4) were synthesised via standard chemical transformations starting from 4-azidobenzoic acid and commercially available reagents (FIG. 11). In all cases, semi-preparative HPLC was used to isolate the compounds in high purity. NOTA-PEG₃-ArN₃ was synthesised in 37% yield after the N-hydroxysuccinimide activated ester (NOTA-NHS) was reacted with a pre-synthesised polyethylene glycol (PEG)-functionalised ArN₃ reagent (N₃-PEG₃-NH₂, FIG. 12 [green trace]).

DOTA-PEG₄-ArN₃ was synthesised in 89% yield via the reaction of DOTA-PEG₄-NH₂ with the activated NHS ester, 2,5-dioxopyrrolidin-1-yl-4-azidobenzoate (2). DOTAGA-PEG₄-ArN₃ was produced in 29% after direct coupling of DOTAGA-PEG₄-NH₂ with 4-azidobenzoic acid in the presence of HATU/DIPEA in DMF.

PEG linkers were introduced to increase the space between the chelate and the photoactivatable ArN₃ group. PEG groups also have the additional benefit of increasing water solubility which is a limiting factor for some chelates. However, it is conceivable that shorter linkers or even direct coupling of ArN₃ to one of the carboxylate arms of the chelates would also generate viable photoactive reagents.

Synthesis of Metal Complexes

In addition to the chelates, non-radioactive Ga complexes were produced and characterised by HR-ESI-MS and UHPLC (FIG. 12, red trace). Radiolabelling experiments were monitored by radioactive instant thin layer chromatography (radio-iTLC) and radioactive UHPLC. Experiments showed that the chelates readily coordinated ⁶⁸Ga³⁺ ions (FIG. 12, blue trace) and that the radioactive complexes co-eluted with the authenticated non-radioactive Ga-complexes as determined via comparison of the retention times (t_{R}/min), and also by standard co-injection methods. For NOTA-PEG₃-ArN₃, formation of [⁶⁸GaNOTA-PEG₃-ArN₃]⁺ was complete in <5 min. at room temperature. In contrast, synthesis of ⁶⁸GaDOTA-PEG₄-ArN₃ and [⁶⁸Ga DOTAGA-PEG₄-ArN₃], from the DOTA (3) and DOTAGA (4) derivatives, respectively, required heating to 70° C. for approximately 5 min to affect complete complexation. A potentially useful feature of this set of chelates is that the varying number of carboxylate groups in NOTA-PEG₃-ArN₃ (1), DOTA-PEG₄-ArN₃ (3) and DOTAGA-PEG₄-ArN₃ (4), means that, under physiological conditions (pH 7.4), the Ga³⁺ (and other 3+ metal ion) complexes will have different overall charges ranging from +1 to −1.

Testing of the Photochemical Reactivity of the Metal Complexes

Following successful radiolabelling experiments on the chelates, the photochemical reactivity of the ⁶⁸Ga-complexes was tested. Samples of [⁶⁸GaNOTA-PEG₃-ArN₃]⁺, [⁶⁸GaDOTA-PEG₄-ArN₃] and [⁶⁸GaDOTAGA-PEG₄-ArN₃] were irradiated using an intense light-emitting diode (LED, 365 nm, 10-30 min, room temperature). Subsequent radio-iTLC and radio-UHPLC analysis confirmed that the radioactive complexes reacted rapidly under UV-irradiation to give essentially a single major new radioactive species (FIG. 12, black trace). The photodegraded products each eluted with shorter retention times indicating that the new species are more hydrophilic than the parent complexes. Under the conditions employed, photoactivation of ArN₃ produces the short-lived arylnitrene species in the singlet (¹A₂) ground state. When the ortho-positions with respect to the N atom are accessible, rapid intramolecular rearrangement of the singlet arylnitrene occurs to give a benzazirine species that undergoes ring expansion to yield a ketenimine intermediate. In the absence of more powerful nucleophiles (primary or secondary amines), it has been shown that the ketenimines intermediate reacts with water to give the more polar azepin-2-ol species (or equivalent tautomers) as the major photodegradation product. Experimental data on photochemical reactivity of [⁶⁸GaNOTA-PEG₃-ArN₃]⁺, [⁶⁸GaDOTA-PEG₄-ArN₃] and [⁶⁸GaDOTAGA-PEG₄-ArN₃] are consistent with this mechanism.

Radiolabelling of Target Molecules

The two-step, one-pot photoradiochemical approach for radiolabelling a monoclonal antibody, and structures of the three products is shown in Scheme 2.

Standard ⁶⁸Ga³⁺ radiochemistry is not perfectly compatible with the photochemical conjugation step because the complexation reaction is performed under acidic conditions (pH˜4.4, NaOAc buffer). In contrast, the photochemical conjugation proceeds most efficiently under slightly basic conditions where the nucleophilicity of the lysine side-chain is increased via deprotonation of the primary ε-NH₂ amine (pKa ˜10.5). For this reason, the chelates were pre-radiolabelled with [⁶⁸Ga][Ga(H₂O)6]³⁺ before adjusting the pH in situ to >7.5 using NaHCO₃ solution. Complex formation was monitored by radio-iTLC and radio-size-exclusion chromatography (SEC) UHPLC. After complete complexation, an aliquot of pre-purified monoclonal antibody was added with an initial chelate-to-monoclonal antibody ratio of ˜10-to-1. Reaction mixtures were then irradiated for 15 min at room temperature. Aliquots of the crude reaction mixtures were analysed by radio-iTLC, manual size-exclusion chromatography (PD-10-SEC) and radio-SEC-UHPLC. In addition, a fraction was purified by preparative PD-10 and spin-centrifugation methods to measure the absolute radiochemical yield (RCY), radiochemical purity (RCP) and molar activities of the purified ⁶⁸Ga-radiolabelled antibody (FIG. 13). Note, all experiments were performed in triplicate with independent replicates. Starting from either compound NOTA-PEG₃-ArN₃ (1), DOTA-PEG₄-ArN₃ (3) and DOTAGA-PEG₄-ArN₃ (4), ⁶⁸Ga-radiolabelled antibody was produced in crude radiochemical yields of around 16-18%, as measured by analytical PD-10-SEC, and 11-16%, as measured by radioactive SEC-UHPLC (FIG. 13). Based on the known initial concentrations of the reagents, the estimated final chelate-to-monoclonal antibody ratios were in the range 1.1 to 1.8. For the radiochemical synthesis of [⁶⁸Ga]GaNOTA-azepin-antibody, the purified sample was isolated in PBS with a decay-corrected RCY of 10.1 ±0.7% (n=3), a RCP >95%, and a molar activity, Aₘ of 0.46±0.09 MBq nmol⁻¹ of protein (n=3; the protein concentration was remeasured after radioactive decay to obtain an accurate value).

One-pot photoradiochemistry using NOTA-PEG₃-ArN₃ (1)

To a solution of NOTA-PEG₃-ArN₃ (1) (50 μg, 7.68×10⁻⁸ mol, 1.02 mM) buffered with NaOAc (0.53 M, pH4.4) was added [⁶⁸Ga][Ga(H₂O)₆]Cl₃(aq.) stock solution (30.8±4.3 MBq, n=3) resulting in a total reaction volume of 75 μL. Reactions were monitored by radio-iTLC. Formation of [68Ga][GaNOTA-PEG₃-ArN₃]⁺ (⁶⁸Ga-1⁺) was complete after 5 min. incubation at 23° C. with radiochemical conversion (RCC) >99% (n=3, R_{f}=0.03−0.19 on iTLC). The pH of the reaction mixture was then adjusted to >7.5 by the addition of an aqueous solution of NaHCO₃ (1.0 M, 50 μL added). After adjusting the pH, an aliquot of pre-purified monoclonal antibody (1.015 mg, 7.00×10⁻⁹ mol, reaction concentration=6.3 mg/mL) was added to give an initial chelate-to-antibody ratio of 11.0 at the start of the photochemical conjugation step (total reaction volume ˜160 μL). The reaction mixture was then irradiated using the LED (100% intensity, 365 nm) for 15 min. at room temperature without stirring. Aliquots of this crude reaction mixture were then analysed by using radio-iTLC, PD-10-SEC and SEC-UHPLC analysis.

Radio-iTLC analyses of the crude reactions after irradiation showed that ˜30% (n=3) of the radioactivity was bound to the antibody (R_{f}=0.0). Note: integration of these radio-iTLC data is unreliable because the radiolabelled antibody fraction partially overlaps with the peak associated with ⁶⁸Ga-1⁺ and the photodegraded ⁶⁸Ga-1⁺ species (R_{f}=0.03−0.19). Nevertheless, analytical PD-10-SEC measurements on the crude reaction mixtures confirmed this observation with an estimated RCP of 15.9±1.8% (n=3). Equivalent decay corrected SEC-UHPLC measurements indicated that the radiolabelled fraction of [⁶⁸Ga]GaNOTA-azepin-antibody in the crude mixture was 15.5±1.5% (n=3).

Crude reaction mixtures were then purified by preparative PD-10-SEC eluting with PBS (collecting only the high purity 0.0-1.6 mL fraction). Prior to analysis, samples were concentrated using an Amicon Ultra-4 mL centrifugal filter (Millipore, 30 kDa MWCO, 4000 RPM, ˜10 min.). The purified and formulated [⁶⁸Ga]GaNOTA-azepin-antibody products (pH7.4) were obtained in <25 min. with decay corrected radiochemical yields (RCY) of 10.1±0.7% (n=3). The estimated lower limit on the molar activity (Aₘ/[MBq/nmol] of protein) of the formulated [⁶⁸Ga]GaNOTA-azepin-antibody samples was 0.46±0.09 MBq/nmol (n=3, remeasured protein concentration). Purified products were then reanalysed by radio-iTLC, analytical PD-10-SEC and SEC-UHPLC. The RCP of purified [⁶⁸Ga]GaNOTA-azepin-antibody was >99% (n=3) by radio-iTLC, 91.3±4.4% (n=3) by analytical PD-10-SEC, and 95.2±2.0% (n=3) by SEC-UHPLC.

One-Pot Photoradiochemistry Using DOTA-PEG₄-ArN₃ (3)

To a solution of DOTA-PEG₄-ArN₃ (3) (60 μg, 7.81×10⁻⁸ mol, 1.03 mM) buffered with NaOAc (0.53 M, pH4.4) was added [⁶⁸Ga][Ga(H₂O)₆]Cl₃(aq.) stock solution (31.6±1.1 MBq, n=3) resulting in a total reaction volume of 76 μL. Reactions were monitored by radio-iTLC. Formation of [⁶⁸Ga]GaDOTA-PEG₄-ArN₃ (⁶⁸Ga-3) was complete after 10 min. incubation at 70° C. with radiochemical conversion (RCC)>99% (n=3, R_{f} =0.06-0.21 on iTLC). The pH of the reaction mixture was then adjusted to >7.5 by the addition of an aqueous solution of NaHCO₃ (1.0 M, 50 μL added). After adjusting the pH, an aliquot of pre-purified monoclonal antibody (1.015 mg, 7.00×10⁻⁹ mol, reaction concentration=6.3 mg/mL) was added to give an initial chelate-to-antibody ratio of 11.2 at the start of the photochemical conjugation step (total reaction volume ˜160 μL). The reaction mixture was then irradiated using the LED (100% intensity, 365 nm) for 15 min. at room temperature without stirring. Aliquots of this crude reaction mixture were then analysed by using radio-iTLC, PD-10-SEC and SEC-UHPLC analysis.

Radio-iTLC analyses of the crude reactions after irradiation showed that ˜30% (n=3) of the radioactivity was bound to the antibody (R_{f}=0.0). Note: integration of these radio-iTLC data is unreliable because the radiolabelled antibody fraction partially overlaps with the peak associated with ⁶⁸Ga-3 and the photodegraded ⁶⁸Ga-3 species (R_{f}=0.06−0.21). Nevertheless, analytical PD-10-SEC measurements on the crude reaction mixtures confirmed this observation with an estimated RCP of 16.2±0.3% (n=3). Equivalent decay corrected SEC-UHPLC measurements indicated that the radiolabelled fraction of [⁶⁸Ga]GaDOTA-azepin-antibody in the crude mixture was 12.7±3.2% (n=3).

Crude reaction mixtures were then purified by preparative PD-10-SEC eluting with PBS (collecting only the high purity 0.0-1.6 mL fraction). Prior to analysis, samples were concentrated using an Amicon Ultra-4 mL centrifugal filter (Millipore, 30 kDa MWCO, 4000 RPM, ˜10 min.). The purified and formulated [⁶⁸Ga]GaDOTA-azepin-antibody products (pH7.4) were obtained in <30 min. with decay corrected radiochemical yields (RCY) of 8.3±1.4% (n=3). The estimated lower limit on the molar activity (Aₘ/[MBq/nmol] of protein) of the formulated [⁶⁸Ga]GaDOTA-azepin-antibody samples was 0.37±0.08 MBq/nmol (n=3, remeasured protein concentration). Purified products were then reanalysed by radio-iTLC, analytical PD-10-SEC and SEC-UH PLC. The RCP of purified [⁶⁸Ga]GaDOTA-azepin-antibody was >99% (n=3) by radio-iTLC, 90.7±1.1% (n=3) by analytical PD-10-SEC, and 93.0±3.0% (n=3) by SEC-UHPLC.

One-Pot Photoradiochemistry Using DOTAGA-PEG₄-ArN₃ (4)

To a solution of DOTAGA-PEG₄-ArN₃ (4) (60 μg, 7.14×10⁻⁸ mol, 0.94 mM) buffered with NaOAc (0.53 M, pH4.4) was added [⁶⁸Ga][Ga(H₂O)₆]Cl₃(aq.) stock solution (31.6±3.0 MBq, n=3) resulting in a total reaction volume of 76 μL. Reactions were monitored by radio-iTLC. Formation of [⁶⁸Ga][GaDOTAGA-PEG₄-ArN₃]⁻ (⁶⁸Ga-4⁻) was complete after 10 min. incubation at 70° C. with radiochemical conversion (RCC) >99% (n=3, R_{f}=0.06−0.22 on iTLC). The pH of the reaction mixture was then adjusted to >7.5 by the addition of an aqueous solution of NaHCO₃ (1.0 M, 50 μL added). After adjusting the pH, an aliquot of pre-purified monoclonal antibody (1.015 mg, 7.00×10⁻⁹ mol, reaction concentration=6.3 mg/mL) was added to give an initial chelate-to-antibody ratio of 10.2 at the start of the photochemical conjugation step (total reaction volume ˜160 λL). The reaction mixture was then irradiated using the LED (100% intensity, 365 nm) for 15 min. at room temperature without stirring. Aliquots of this crude reaction mixture were then analysed by using radio-iTLC, PD-10-SEC and SEC-UHPLC analysis.

Radio-iTLC analyses of the crude reactions after irradiation showed that ˜30% (n=3) of the radioactivity was bound to the antibody (R_{f}=0.0). Note: integration of these radio-iTLC data is unreliable because the radiolabelled antibody fraction partially overlaps with the peak associated with ⁶⁸Ga-4⁻ and the photodegraded ⁶⁸Ga-4⁻ species (R_{f}=0.06−0.22). Nevertheless, analytical PD-10-SEC measurements on the crude reaction mixtures confirmed this observation with an estimated RCP of 18.3±0.7% (n=3). Equivalent decay corrected SEC-UHPLC measurements indicated that the radiolabelled fraction of [⁶⁸Ga]GaDOTAGA-azepin-antibody in the crude mixture was 11.1±0.2% (n=3).

Crude reaction mixtures were then purified by preparative PD-10-SEC eluting with PBS (collecting only the high purity 0.0-1.6 mL fraction). Prior to analysis, samples were concentrated using an Amicon Ultra-4 mL centrifugal filter (Millipore, 30 kDa MWCO, 4000 RPM, —10 min.). The purified and formulated [⁶⁸Ga]GaDOTAGA-azepin-antibody products (pH7.4) were obtained in <30 min. with decay corrected radiochemical yields (RCY) of 9.2±0.6% (n=3). The estimated lower limit on the molar activity (Aₘ/[MBq/nmol] of protein) of the formulated [⁶⁸Ga]GaDOTAGA-azepin-antibody samples was 0.37±0.07 MBq/nmol (n=3, remeasured protein concentration). Purified products were then reanalysed by radio-iTLC, analytical PD-10-SEC and SEC-UHPLC. The RCP of purified [⁶⁸Ga]GaDOTAGA-azepin-antibody was >99% (n=3) by radio-iTLC, 92.2±1.0% (n=3) by analytical PD-10-SEC, and 92.2±1.8% (n=3) by SEC-UHPLC.

Experimental

The experiments described in this specification may be performed with any antibody or antibody fragment that comprises a free amine or thiol moiety such as cetuximab, bevacizumab, trastuzumab, panitumumab, ibritumomab tiuxetan, onartuzumab, J591, fresolimumab, rituximab, brentuximab, lumretuzumab, U36, R1507, ranibizumab, DN30, 7E11, particularly trastuzumab. As described above, the photoconjugation requires an amine or thiol moiety such as in the side chain of the amino acids lysine or cysteine. The experiments described herein using an antibody were performed with trastuzumab.

General Details

Unless otherwise stated, all chemicals were of reagent grade and purchased from SigmaAldrich (St. Louis, Mo.), Merck (Darmstadt, Germany), Tokyo Chemical Industry (Eschborn, Germany), abcr (Karlsruhe, Germany) or CheMatech (Dijon, France). Water (>18.2 MΩ·cm at 25° C., Puranity TU 3 UV/UF, VWR International, Leuven, Belgium) was used without further purification. Solvents for reactions were of reagent grade, and where necessary, were dried over molecular sieves. Evaporation of the solvents was performed under reduced pressure by using a rotary evaporator (Rotavapor R-300, Buchi Labortechnik AG, Flawil, Switzerland) at the specified temperature and pressure. If the antibody is not specified otherwise, the experiments described herein were performed using the antibody trastuzumab.

¹H and ¹³C NMR spectra were measured in deuterated solvents on a Bruker AV-400 (¹H: 400 MHz, ¹³C: 100.6 MHz) or a Bruker AV-500 (¹H: 500 MHz, ¹³C: 125.8 MHz) spectrometer. Chemical shifts (6) are expressed in parts per million (ppm) relative to the resonance of the residual solvent peaks, for example, with DMSO 6_{H}=2.50 ppm and δ_{C}=39.5 ppm with respect to tetramethylsilane (TMS, δ_{H} and δ_{C}=0.00 ppm). Coupling constants (J) are reported in Hz. All resonances were assigned by using a combination of 1D and 2D NMR (HSQC, COSY) spectra. Peak multiplicities are abbreviated as follows: s (singlet), d (doublet), dd (doublet of doublets), t (triplet), q (quartet), m (multiplet), and br (broad).

High-resolution electrospray ionisation mass spectra (HR-ESI-MS) were measured by the mass spectrometry service at the Department of Chemistry, University of Zurich.

Column chromatography was performed by using Merck silica gel 60 (63-200 μm) with eluents indicated in the experimental section. Standard thin-layer chromatography (TLC) for synthesis employed Merck TLC plates silica gel 60 on an aluminium base with the indicated solvent system. The spots on TLC were visualised either by UV/visible light (254 nm) or by staining with KMnO₄.

Semi-preparative high-performance liquid chromatography (HPLC) purifications were performed using a Rigol HPLC system (Contrec AG, Dietikon, Switzerland) equipped with a 018 reverse-phase column (VP 250/21 Nucleodur C18 HTec, 21 mm ID×250 mm, 5 μm) using a flow rate of 8 mL min⁻¹ with a linear gradient of solvent A (distilled H₂O containing 0.1% TFA) and B (MeOH): t=0-3 min., 60% A; t=25-30 min., 5% A; t=33-38 min., 60% A. Electronic absorption was measured at 254 nm.

Analytical ultra-high-performance liquid chromatography (UHPLC) experiments were performed using two separate Hitachi Chromaster Ultra Rs systems fitted with either a reverse phase VP 250/4 Nucleodur C18 HTec (4 mm ID×250 mm, 5 μm) column or a reverse phase Acquity UPLC column (BEH C18, 1.7 μm, 2.1 mm ID×50 mm). One of these systems was also connected to a radioactivity detector (FlowStar² LB 514, Berthold Technologies, Zug, Switzerland) equipped with a 20 μL PET cell (MX-20-6, Berthold Technologies) for analysing radiochemical reactions. Proteins were analysed by using the same UHPLC system equipped with a size-exclusion column (Enrich SEC 70 column: 24 mL volume, 10±2 μm particle size, 10 mm ID×300 mm, Bio-Rad Laboratories, Basel, Switzerland). UHPLC using the Acquity column used a flow rate of 0.6 mL min⁻¹ with a linear gradient of solvent A (distilled H₂O containing 0.1% TFA) and B (acetonitrile): t=0-0.5 min., 30% A; t=9.5 min., 0% A; t=10 min., 0% A. Electronic absorption was measured at 254 nm.

Analytical high performance liquid chromatography (HPLC) experiments for photodegradation kinetics were performed using a Hitachi Chromaster system equipped with a reverse phase column (Reproshell 100 Dr. Maisch C18, 2.8 μm, 75×4.6 mm) using a flow rate of 1.5 mL min⁻¹ with a linear gradient of solvent A (distilled H₂O containing 0.1% HCOOH) and B (acetonitrile): t=0 min., 95% A; t=5.8 min., 0% A; t=6.8 min., 0% A; t=7.3 min., 90% A. Electronic absorption was measured at 260 nm.

Electronic absorption spectra were recorded using a Nanodrop™ One^{C} Microvolume UV-Vis Spectrophotometer (ThermoFisher Scientific, supplied by Witec AG, Sursee, Switzerland). Protein concentration was determined in accordance with the manufacturers protocol.

Photochemistry

Photochemical conjugation experiments were performed in transparent glass vials at the indicated concentrations. Stock solutions were prepared in H₂O (antibody and DFO-ArN₃ [1]).

Photochemical reactions were stirred gently using a magnetic stir bar. Detail procedure and reaction times are indicated in the experimental section. Irradiations used three light sources. For pre-conjugation experiments, a high-powered Rayonet reactor^{[1]} (350 nm, 16×8 W Sylvania BLB-lamps, 10 cm diameter) was used. For kinetic studies and for simultaneous one-pot photoradiochemical labelling reactions, portable, high-powered, light-emitting diodes (LEDs at either 365 nm or 395 nm) were used. The LED intensity was adjusted using a UV-LED controller (Opsytec Dr. Grobel GmbH, Ettlingen, Germany), where 100% corresponded to a power of approximately 263 mW and 355 mW for the 365 nm and 395 nm sources, respectively. LED intensity was measured using a S470C Thermal Power Sensor Head, Volume Absorber, 0.25-10.6 μm, 0.1mW-5W, ∅15 mm. Total irradiance power of the

Rayonet reactor was estimated to be approximately 92 mW (approximately 300 mW/cm³). Note that calculation of exact power incident to the reaction is non-trivial because it depends on the specific geometry of the experiment. The temperature of all photochemical conjugation reactions was typically 23±2° C. The Rayonet reactor had an experimentally measured λₘₐₓ at 368 nm with full-width at half-maximum (FWHM) value of 16.0 nm. The LED (365 nm) had a maximum emission intensity at 364.5 nm (FWHM of 9.1 nm). The LED (395 nm) had a maximum emission intensity at 389.9 nm (FWHM of 9.1 nm).

Radioactivity and Radioactive Measurements

All instruments for measuring radioactivity were calibrated and maintained in accordance with previously reported routine quality control procedures.^{[2]} [⁸⁹Zr][Zr(C₂O₄)₄]⁴⁻(aq.) was obtained as a solution in ˜1.0 M oxalic acid from PerkinElmer (Boston, Ma., manufactured by the BV Cyclotron VU, Amsterdam, The Netherlands) and was used without further purification. Radioactive reactions were monitored by using instant thin-layer chromatography (radio-iTLC). Glass-fibre iTLC plates impregnated with silica-gel (iTLC-SG, Agilent Technologies) were developed in using aqueous mobile phases containing either EDTA (50 mM, pH7.1) or DTPA (50 mM, pH7.4) and were analysed on a radio-TLC detector (SCAN-RAM, LabLogic Systems Ltd, Sheffield, United Kingdom). Radiochemical conversion (RCC) was determined by integrating the data obtained by the radio-TLC plate reader and determining both the percentage of radiolabelled product (R_{f}=0.0) and ‘free’ ⁸⁹Zr (R_{f}=1.0; present in the analyses as either [⁸⁹Zr]Zr(EDTA) or [⁸⁹Zr]Zr(DTPA). Integration and data analysis were performed by using the software Laura version 5.0.4.29 (LabLogic). Appropriate background and decay corrections were applied as necessary. Radiochemical purities (RCPs) of labelled protein samples were determined by size-exclusion chromatography (SEC) using two different columns and techniques. The first technique used an automated size-exclusion column (Bio-Rad Laboratories, ENrich SEC 70, 10±2 μm, 10 mm ID×300 mm) connected to a UHPLC device (Hitachi ChromasterUltra Rs, VWR International, Leuven, Belgium) equipped with a UV/visible diode array detector (absorption measured at 220, 254 and/or 280 nm) as well as a radioactivity detector (FlowStar² LB 514, Berthold Technologies, Zug, Switzerland). Isocratic elution with phosphate buffered saline (PBS, pH7.4) was used. The second method used a manual procedure involving size-exclusion column chromatography using a PD-10 desalting column (Sephadex G-25 resin, 85-260 μm, 14.5 mm ID×50 mm, >30 kDa, GE Healthcare). For analytical procedures, PD-10 columns were eluted with sterile saline or PBS. A total of 40×200 μL fractions were collected up to a final elution volume of 8 mL. Note that the loading/dead-volume of the PD-10 columns is precisely 2.50 mL which was discarded prior to aliquot collection. For quantification of radioactivity, each fraction was measured on a gamma counter (HIDEX Automatic Gamma Counter, Hidex AMG, Turku, Finland) using an energy window between 480-558 keV for ⁸⁹Zr (511 keV emission) and a counting time of 30 s. Appropriate background and decay corrections were applied throughout. PD-10 SEC columns were also used for preparative purification and reformulation of radiolabelled products by collecting a fraction of the eluate corresponding to the high molecular weight protein (>30 kDa fraction eluted in the range between 0.0 to 1.6 mL as indicated for each experiment).

Stability Studies

The stability of [⁸⁹Zr]ZrDFO-azepin-antibody with respect to change in radiochemical purity due to loss of radioactivity from the protein fraction was investigated in vitro by incubation in human serum. Aliquots of [⁸⁹Zr]ZrDFO-azepin-antibody (250 μL, 81.5 μg, 0.54 nmol, 6.59 MBq, Aₘ ˜12.1 MBq/nmol) were added to human serum (400 μL) giving a total reaction volume of 650 μL. Solutions were incubated at 37° C. and SEC-UHPLC measurements recorded at the specified time points up to 45 h. The stability was monitored by quantifying the radioactivity associated with intact [⁸⁹Zr]ZrDFO-azepin-antibody from integration of the decay corrected SEC-UHPLC radioactive chromatograms.

Synthesis and Chemical Characterisation

Chemical syntheses were performed in accordance with Scheme 2.

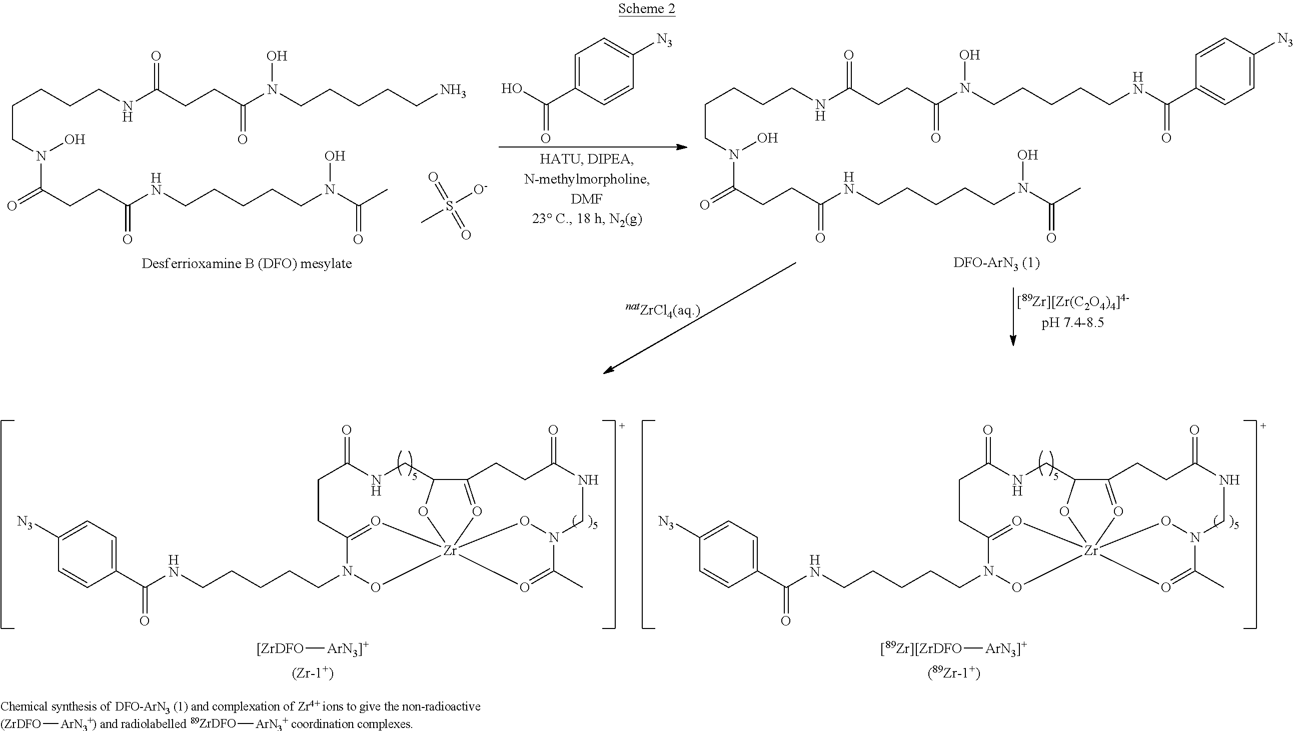

All reactions involving photosensitive compounds were performed in the dark. The IgG₁ antibody component was purified from an antibody preparation by spin column centrifugation (4000 RPM, 3×15 min., 1×20 min.) by using a membrane filter (Amicon Ultra-4 mL centrifugal filter, Millipore, 10 kDa MWCO). Briefly, aliquots of the antibody preparation (60 mg) were washed with H₂O (4×4 mL) at room temperature and concentrated before use.

After concentration, protein samples were removed from the centrifugation filter by rinsing with water (500 μL) and the protein concentration was determined using a Nanodrop™ One^{C} Microvolume UV-Vis Spectrophotometer. Typically, 25-30 mg of protein was obtained and samples were aliquoted into Eppendorf tubes and stored at −20° C. for future use.

Synthesis of Desferrioxamine-p-arylazide, DFO-ArN₃ (1)

A solution of 4-azidobenzoic acid (206 mg, 1.26 mmol), HATU (506 mg, 1.33 mmol) and N,N-diisopropylethylamine (DIPEA, 130 μL) in dry DMF (8 mL) was stirred at room temperature for 40 min. Then desferrioxamine B mesylate (DFO, 407 mg, 0.725 mmol) was added to the mixture along with additional DIPEA (95 μL) and N-methylmorpholine (250 μL). After stirring at room temperature for 80 h, the mixture was transferred to a single-necked round bottom flask (100 mL) and the solvent was evaporated under reduced pressure (25 mbar). The orange-beige residue was washed by sonication with cold acetone (6×7 mL, −20 ° C.) and ice cold H₂O (4×7 mL). Note that between each washing step, the solid residue was collected by centrifugation and cooled. Washing with acetone the orange colour and subsequent lyophilisation gave the crude product DFO-ArN₃ (1, 40% yield, 228 mg, 0.291 mmol, estimated 68% purity measured by ¹H NMR) as a white amorphous powder. A portion of the crude product was purified by semi-preparative HPLC and after lyophilisation, purified compound 1 was obtained as a white amorphous powder. (Yield 4%, estimated purity >95% by UHPLC and by ¹H NMR).

Synthesis of [ZrDFO-ArN₃]⁺ (Zr-1⁺)

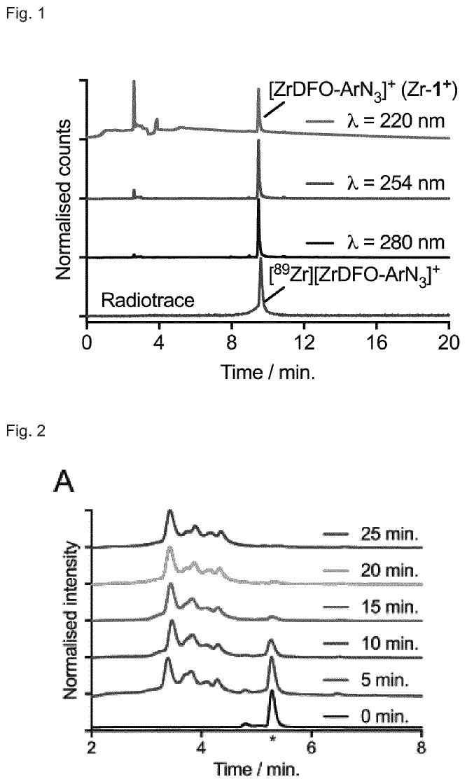

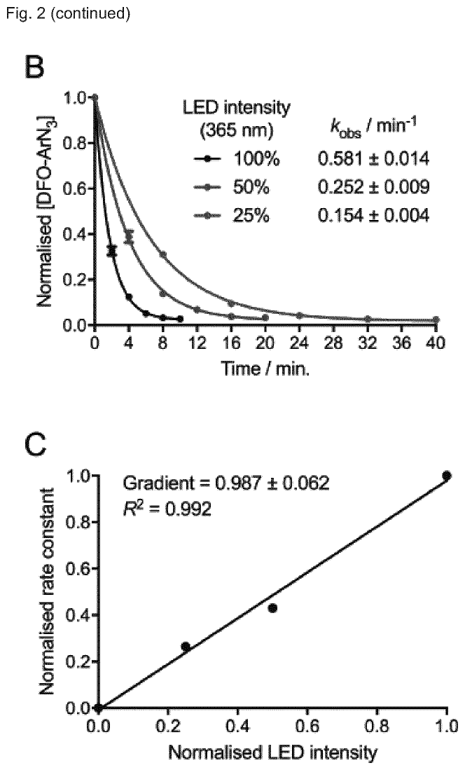

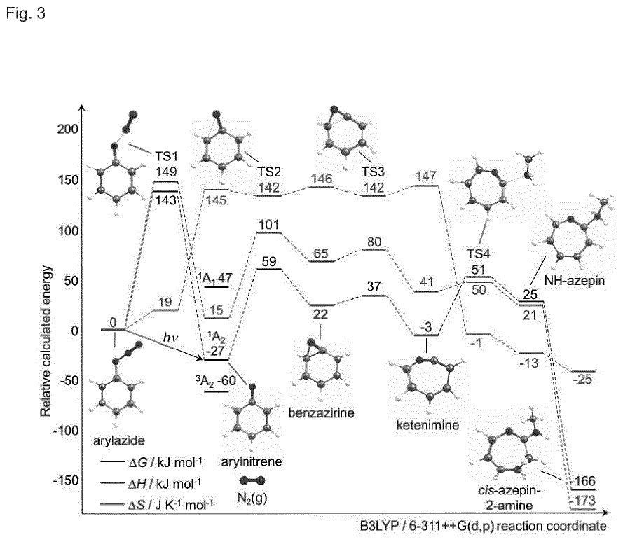

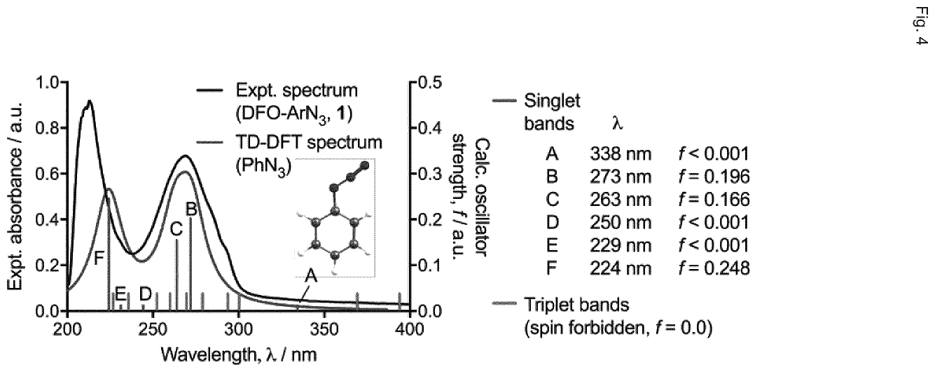

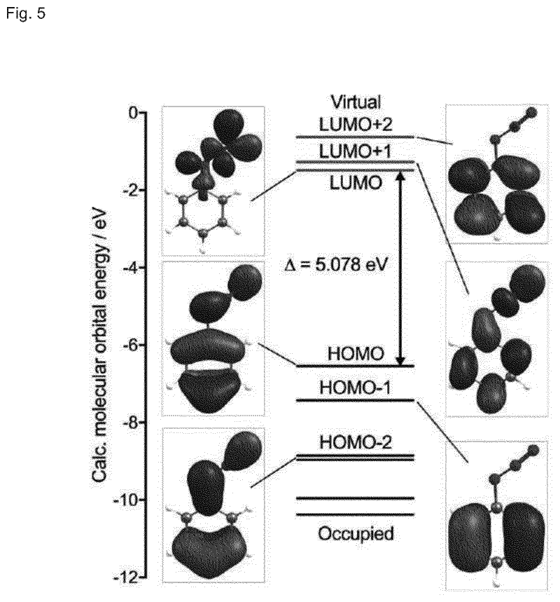

DFO-ArN₃ (1, 0.68 mg, 0.964 μmol was dissolved in a mixture of H₂O (50 μL) and NaOH(aq.) (0.1 M, 30 μL). After dissolution of compound 1, a clear, colourless solution was obtained. Then the pH of the mixture was reduced to ˜8-9 by the addition HCl(aq.) (0.1 M, 2×10 μL). Then an aliquot of ZrCl₄(aq.) (112 μL, 6 M Zr⁴⁺ ions dissolved in 0.1 M HCl(aq.)) was added dropwise. The reaction was monitored by RP-UHPLC and after stirring at room temperature for 2 h, complete conversion was observed. Presence of desired product Zr-1⁺ was confirmed by a single peak in analytical HPLC that gave the expected mass of molecular ion as the base peak in high-resolution electrospray ionisation mass spectrometry (see FIG. 1 main article and Figure S9 below). t_{R} (RP-HPLC)=9.47 min (detection at λ=254 nm). RP-HPLC method: A flow rate of 0.7 mL min⁻¹ with a linear gradient of A (distilled H₂O containing 0.1% TFA) and B (acetonitrile): t=0 min, 90% A; t=20 min, 10% A. HR-ESI(+)-MS (MeOH): m/z calc. for [M⁺] 792.262165, found 792.26200 (100%, Δ=0.43 ppm).

Radiochemistry and Photoradiochemistry

Molar Activity of the [⁸⁹Zr][Zr(C₂O₄)₄]⁴⁻(aq.) Stock Solution

The molar activity of the ⁸⁹Zr-oxalate stock solution was measured by isotopic dilution assays. Briefly, a stock solution of desferrioxamine B mesylate was prepared in water (3.77 mg, MW=656.79 g mol⁻¹, 5.74 μmol, 1.0 mL, [DFO]=5.74 mM) and was diluted to give a secondary solution (2.87 μM). To microcentrifuge tubes (n=3) was added H₂O (90 μL) and an aliquot of the secondary DFO stock solution (10 μL, 0.0287 nmol). Then an aliquot of a neutralised [⁸⁹Zr][Zr(C₂O₄)₄]⁴⁻(aq.) stock solution (see below for details on the neutralisation step) was added to each tube (1.637 MBq). Reactions were vortexed and incubated at room temperature for 90 min. to ensure complete reaction occurred. At the end of the reaction, aliquots were spotted onto iTLC plates and developed using aqueous mobile phase containing DTPA (50 mM, pH7.4) or EDTA (50 mM, pH7.1). Radio-iTLC analysis was used to measure the radiochemical conversion (RCC) with the product [⁸⁹Zr]ZrDFO retained at the baseline (R_{f}=0.0) and either [⁸⁹Zr]Zr(EDTA) or [⁸⁹Zr]Zr(DTPA) eluting at the solvent front (R_{f}=1.0). The experimentally measured molar activity of the [⁸⁹Zr][Zr(C₂O₄)₄]⁴⁻(aq.) stock solution was Aₘ=37.0±0.12 MBq/nmol.

Radiosynthesis and Characterisation of [⁸⁹Zr][ZrDFO-ArN₃]⁺ (⁸⁹Zr-1⁺)

A stock solution of DFO-ArN₃ (1, 0.67 mg, 0.950 μmol) was dissolved in H₂O (50 μL) and NaOH(aq.) (30 μL of a 0.1 M stock solution). The pH of the DFO-ArN₃ solution was reduced to ˜8-9 by the addition of HCl(aq.) (2×10 μL of a 0.1 M stock solution). A stock solution of [⁸⁹Zr][Zr(C₂O₄)₄]⁴⁻ was prepared by adding ⁸⁹Zr radioactivity from the source (68.7 MBq, 70 μL in ˜1.0 M aqueous oxalic acid) to a vial containing water (200 μL). The solution was neutralised and made slightly basic by the addition of aliquots of Na₂CO₃(aq.) (1.0 M stock solution, 55 μL added, final pH ˜8.3-8.5). Caution: Acid neutralisation with Na₂CO₃ releases CO₂(g) and care should be taken to ensure that no radioactivity escapes the microcentrifuge tube. After CO₂ evolution ceased, an aliquot of the neutralised [⁸⁹Zr][Zr(C₂O₄)₄]⁴⁻ solution (20-40 μL, 4.66 MBq) was added to the reaction microcentrifuge vial containing an aliquot of the DFO-ArN₃ stock solution (10 μL, 95 nmol, 9.5 mM) and water (50 μL) giving a clear, colourless solution (pH 7-8). The reaction was vortexed and incubated at room temperature. Reaction progress was monitored by radio-ITLC and complete radiochemical conversion to give of ⁸⁹Zr-1⁺ (R_{f}=0.0) was observed in <10 min. Aliquots of the crude reaction mixture were analysed be radioactive HPLC (FIG. 1). A single peak was observed in the radioactive trace. The identity of the radiolabelled compound ⁸⁹Zr-1⁺ was confirmed by co-injection with an authenticated sample of ^{nat}Zr-1⁺. t_{R} (RP-HPLC)=9.48 min. (detection at λ=220, 254 and 280 nm, FIG. 1). RP-HPLC method: A flow rate of 0.7 mL min⁻¹ with a linear gradient of A (distilled H₂O containing 0.1% TFA) and B (acetonitrile): t=0 min, 90% A; t=20 min, 10% A.

Photochemical Conjugation

General procedure for photochemical conjugation: A stock solution of photoactive ligand was prepared by dissolving DFO-ArN₃ (1, 0.85 mg, 1.21 μmol) in water (50 μL) and NaOH(aq.) (40 μL of a 0.1 M stock solution). Immediately before starting the photochemical conjugation reactions, the pH of the DFO-ArN₃ solution was reduced to ˜9 by the addition of HCl(aq.) (2×10 μL of a 0.1 M stock solution). Note: DFO-ArN₃ (1) is sparingly soluble at high pH but starts to precipitate slowly when the pH decreases below ˜9. For this reason, photochemical reactions should be initiated immediately after adding the HCl and the protein. After adjusting the pH, aliquots of the DFO-ArN₃ stock solution were added to clear 2 mL glass vials equipped with small magnetic stirring bars and containing an aqueous solution of antibody (120 μL, 2.76 mg, 1.84×10⁻⁸ mol, stock protein concentration=23.0 mg/mL) and a variable amount of water (constant total reaction volume=200 μL). The chelate-to-mAb ratio was varied used 5.3-fold (9 μL), 10.7-fold (18 μL) or 26.4-fold (45 μL) excess of DFO-ArN₃ stock solution. The final pH of the solutions was 8-8.5. The reaction mixture was then irradiated for 25 min. using the Rayonet reactor. The irradiated crude mixture was then purified by a three-step procedure. First, the mixture was taken in a 30 kDa MWCO membrane centrifugal filter (Amicon Ultra-4 mL centrifugal filter, Millipore,), concentrated and washed with PBS (2×4 mL) using centrifugation (4000 RPM, ˜15 min). Then the mixture was purified using a preparative PD-10-SEC column (eluted with PBS, collecting the 0.0-1.6 mL fraction immediately after discarding the 2.5 mL column dead volume). In the last step, the fraction from PD-10-SEC was taken in a new 30 kDa MWCO membrane centrifugal filter, washed and concentrated using PBS (2×4 mL) followed by water (2×4 mL) as described in first step. The purified protein was removed from the spin column filter in a final volume of ˜320 μL water. Protein concentration was measured using the Nanodrop. Stock solutions of DFO-azepin-antibody were aliquoted and stored at −20° C.

⁸⁹Zr-Radiolabelling of DFO-azepin-antibody

For animal experiments, the radiochemical synthesis of [⁸⁹Zr]ZrDFO-azepin-antibody was scaled up using a sample of DFO-azepin-antibody prepared from an initial chelate-to-antibody ratio of 26.4-to-1 in the photochemical conjugation reaction. To a microcentrifuge tube was added water (100 μL) and [⁸⁹Zr][Zr(C₂O₄)₄]⁴⁻(aq.) stock solution (70 μL, 88.66 MBq). The oxalic acid was neutralised and made slightly basic by the addition of aliquots of Na₂CO₃(aq.) (˜1.0 M, 55 μL, final pH8.1-8.3). Caution: Acid neutralisation with Na₂CO₃ releases CO₂(g) and care should be taken to ensure that no radioactivity escapes the microcentrifuge tube. After CO₂ evolution ceased, an aliquot of photochemically conjugated DFO-azepin-antibody (125 μL, 8.0 mg/mL, mass=1.0 mg of protein, 6.67 nmol) produced using an initial chelate-to-mAb ratio of 26.4-to-1 was added to the neutralised solution of [⁸⁹Zr][Zr(C₂O₄]⁴⁻(aq.). The pH decreased slightly to 6.6 and was readjusted to pH7.4-7.7 by the addition of Na₂CO₃(aq.) (˜1.0 M, 4 μL). The reaction was mixed gently and then incubated at room temperature for 1 h. The reaction was monitored by radio-iTLC. Control reactions performed in the absence of antibody showed complete formation of [⁸⁹Zr]Zr(EDTA) under the conditions used to develop the iTLC plates with no activity retained at the baseline (R_{f}=0.0). The reaction showed a RCC>95% after the 15 minutes but a slight improvement in RCC occurred by 40 min. (RCC>98%), which remained the same by 60 min. After 1 h, the reaction was quenched by the addition of a small aliquot of EDTA (5 μL, 50 mM, pH7.4) and incubating for a further 5 min. An aliquot of the crude mixture was retained for further analysis and then the major fraction (250 μL) was purified by preparative PD-10-SEC eluting with sterile PBS. All crude and purified mixtures were analysed by radio-iTLC, analytical PD-10-SEC and SEC-UHPLC.

The radiochemical purity (RCP) of the crude sample of [⁸⁹Zr]ZrDFO-azepin-antibody was determined by analytical PD-10-SEC (>98%) as well as SEC-HPLC (>98%). Purification and formulation [⁸⁹Zr]ZrDFO-azepin-antibody (pH7.4) was completed in <5 min. with a decay corrected radiochemical yield (RCY) of >99%, and a final activity concentration of 29.67 MBq/mL. After preparative PD-10-SEC (collecting the 0.0-1.8 mL fraction) the RCP was to >99.5% (measured by analytical PD-10-SEC) and >98% (measured by SEC-UHPLC).

Aliquots of the final [⁸⁹Zr]ZrDFO-azepin-antibody product were then prepared for injection in the normal and blocking groups of animals (n=6 mice/group). Briefly, two aliquots of [⁸⁹Zr]ZrDFO-azepin-antibody (350 μL, ˜10.4 MBq) were added to separate centrifuge tubes. For the normal group dose, the activity was diluted with sterile PBS (1.65 mL) giving a final volume of 2.0 mL. For the blocking group, the activity was diluted with sterile PBS (1.511 mL) and then an aliquot of non-radiolabelled antibody (stock protein concentration=57.7 mg/mL, 0.139 mL, 8.0 mg) was added and the solution mixed gently. A total of 7 syringes (250 μL/each) were drawn for both the normal and blocking formulations. The seventh syringe was used as a standard for accurate quantification of the biodistribution data (vide supra). In addition, aliquots of the normal and blocking formulations were retained and the protein concentration was re-measured using the Nanodrop. The measured molar activities (Aₘ/[MBq/nmol] of protein) of the injectates (decay-corrected to the point of final formulation) were then calculated as 13.7 MBq/nmol for the normal doses and 0.14 MBq/nmol for the blocking doses. The blocking dose contained ˜98-fold higher concentration of mAb than the normal dose.

Chelate Number Estimation

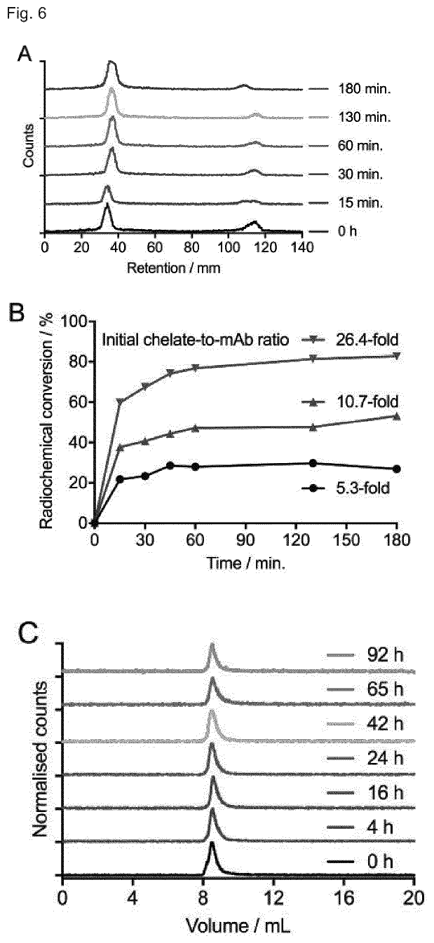

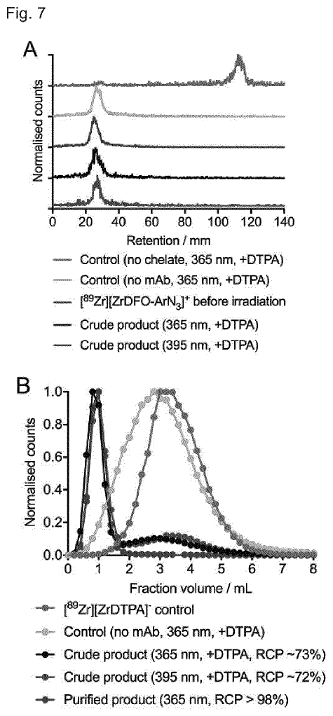

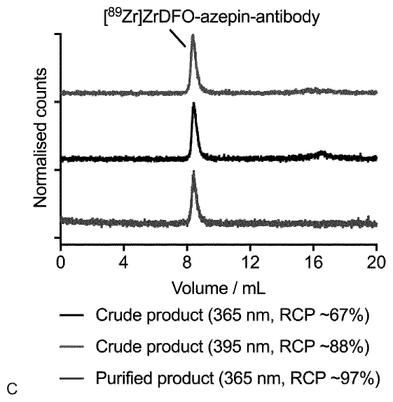

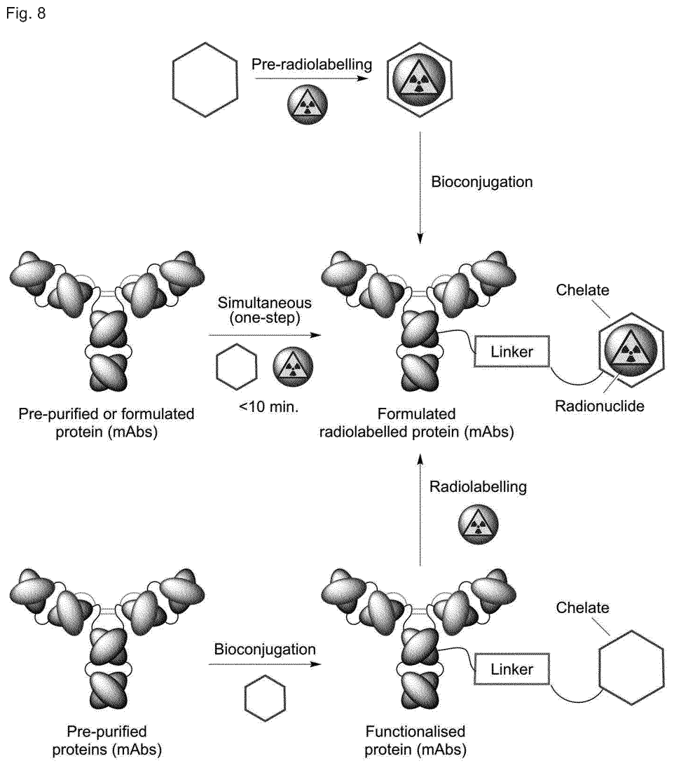

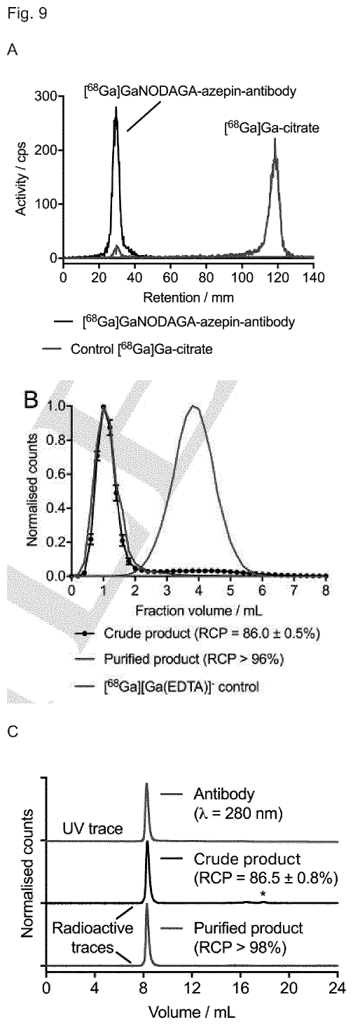

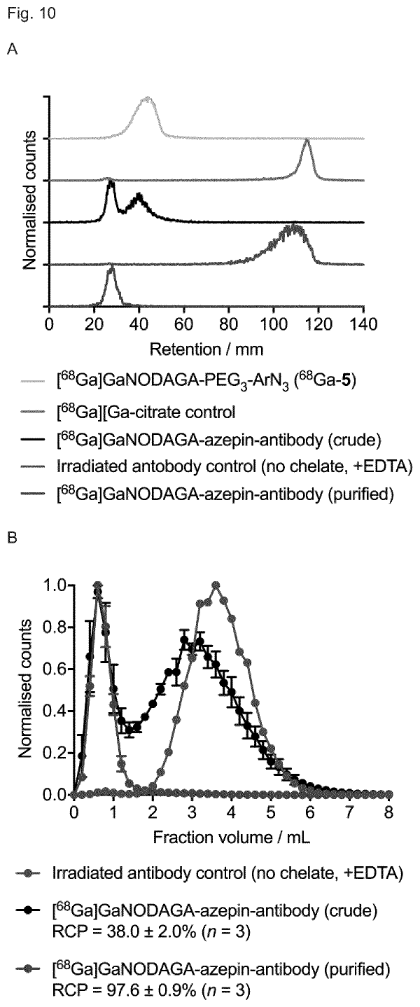

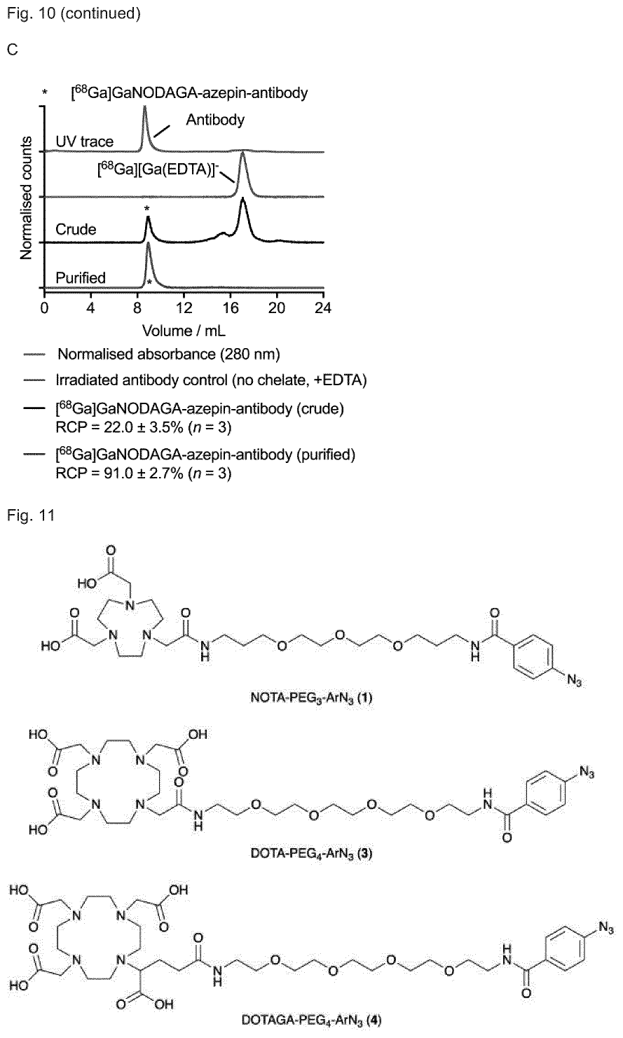

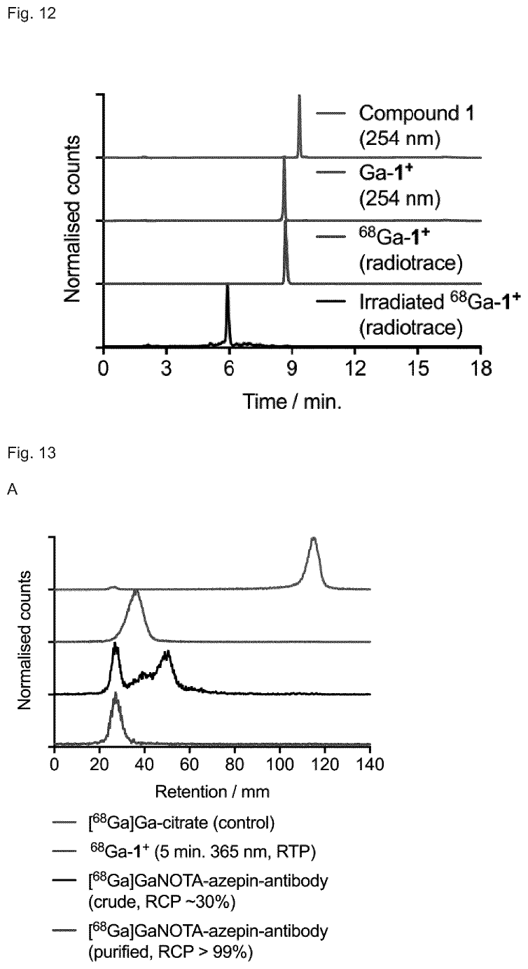

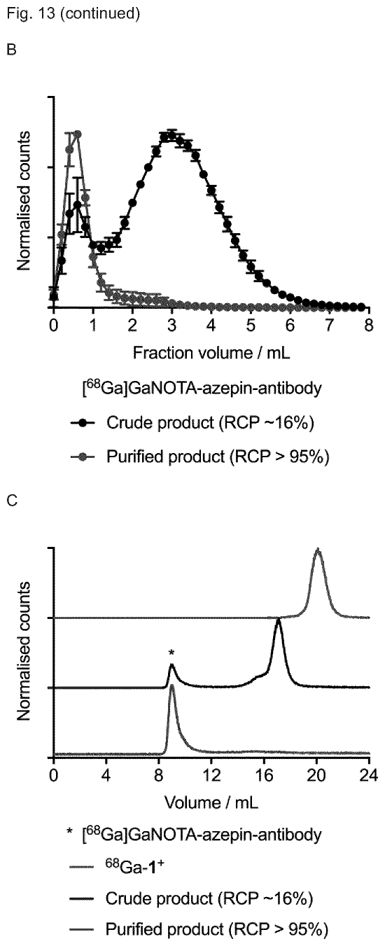

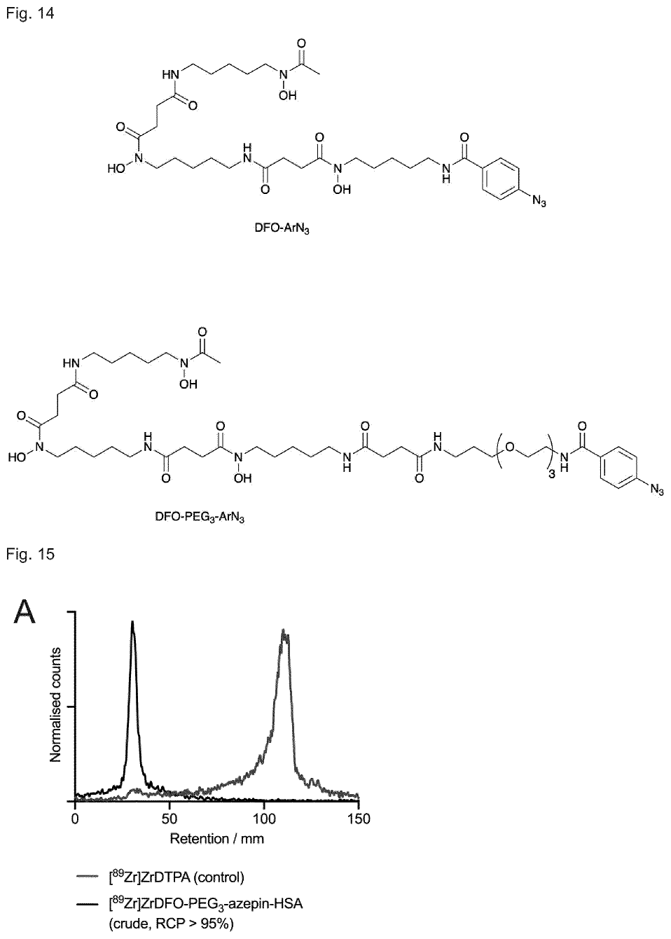

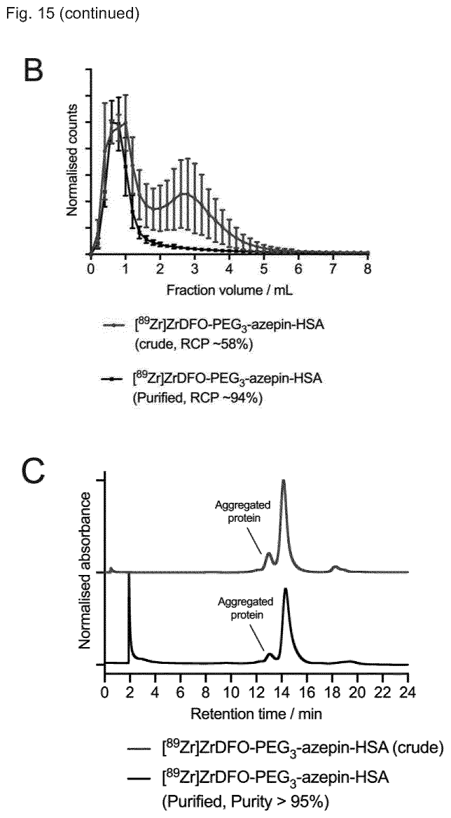

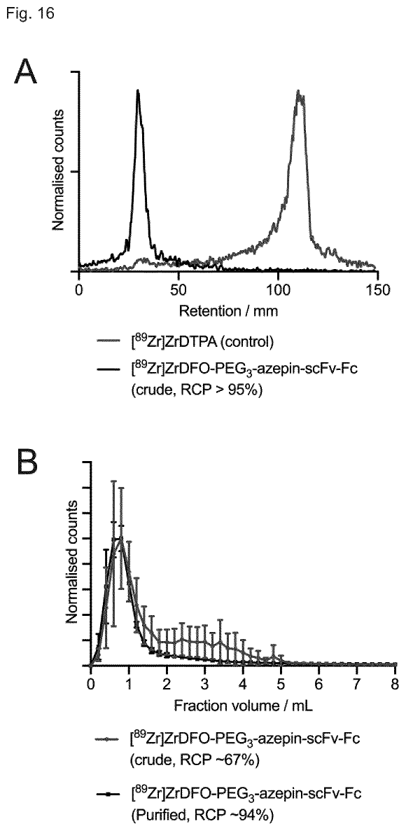

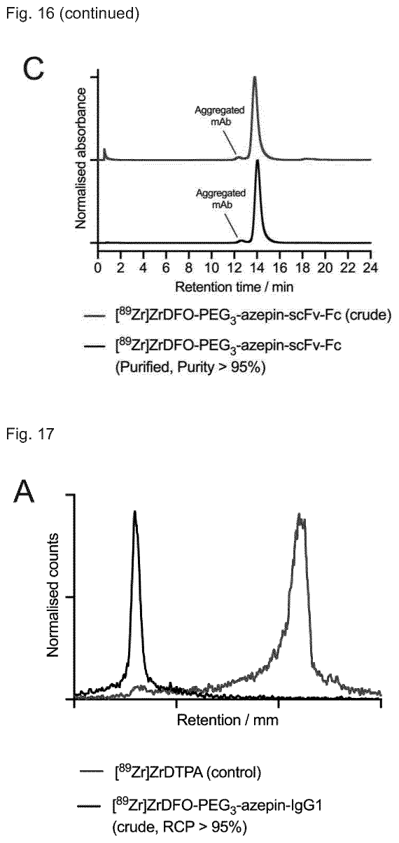

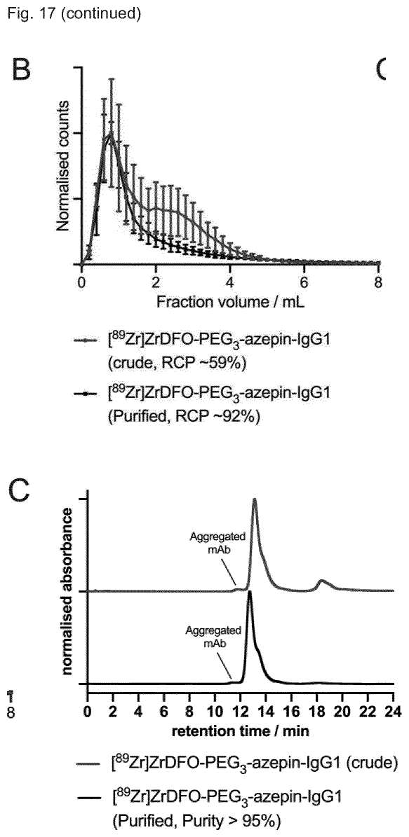

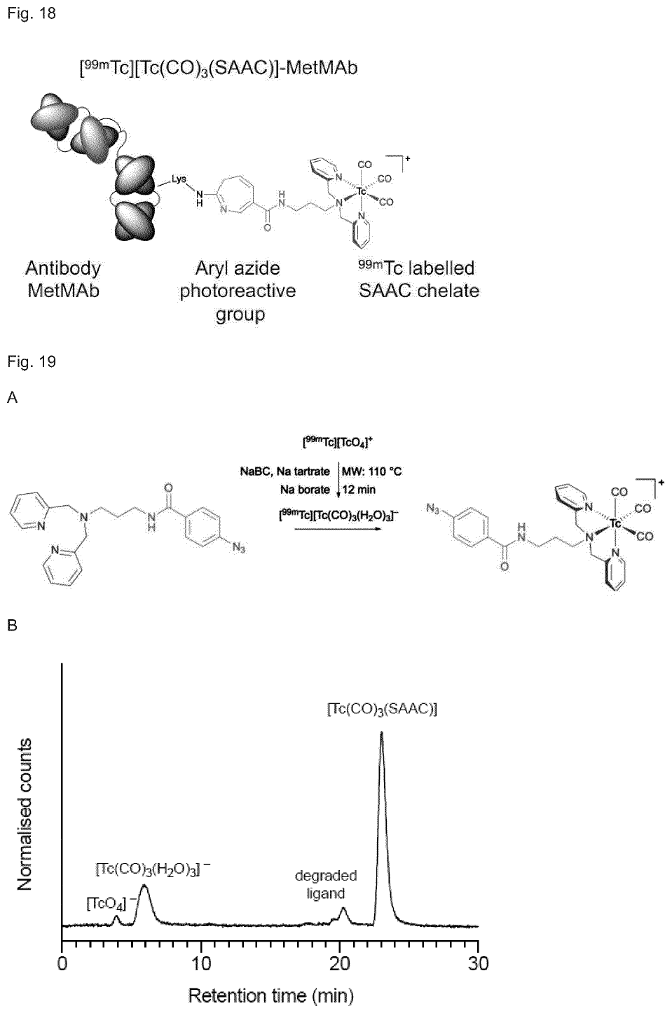

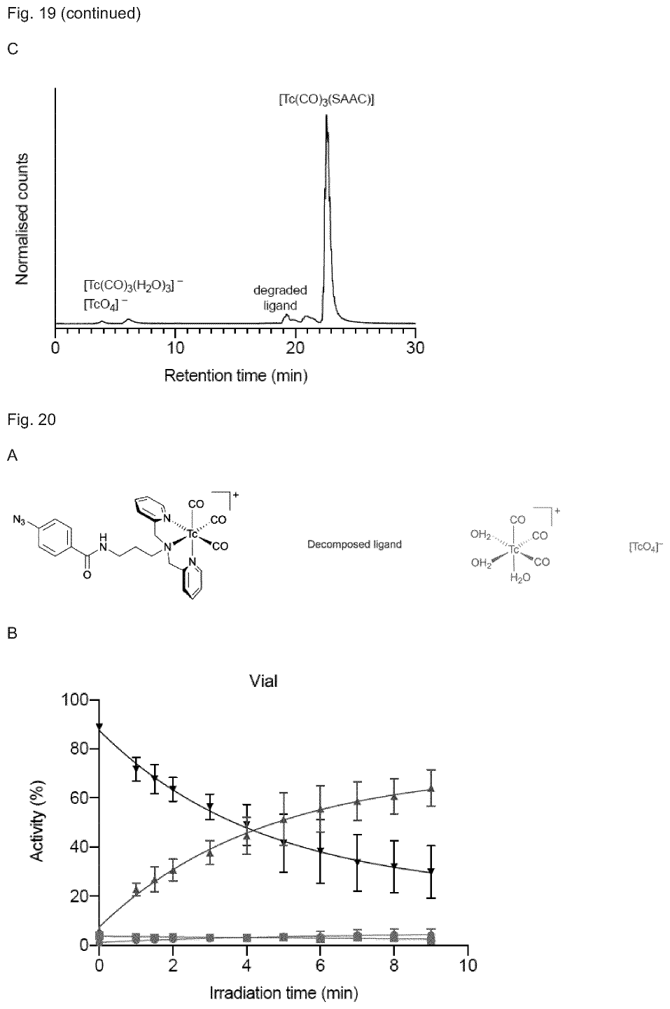

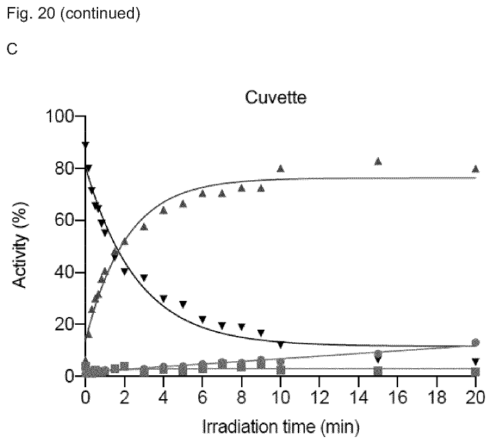

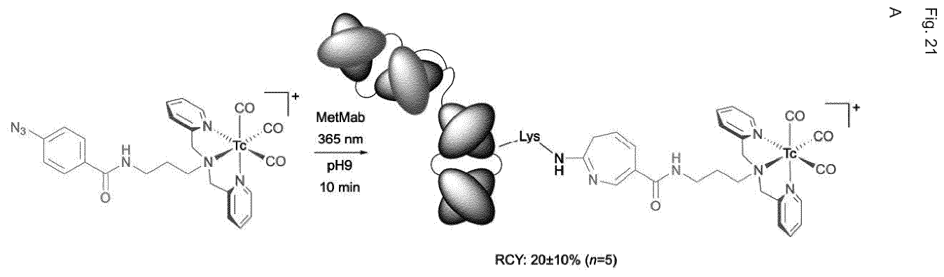

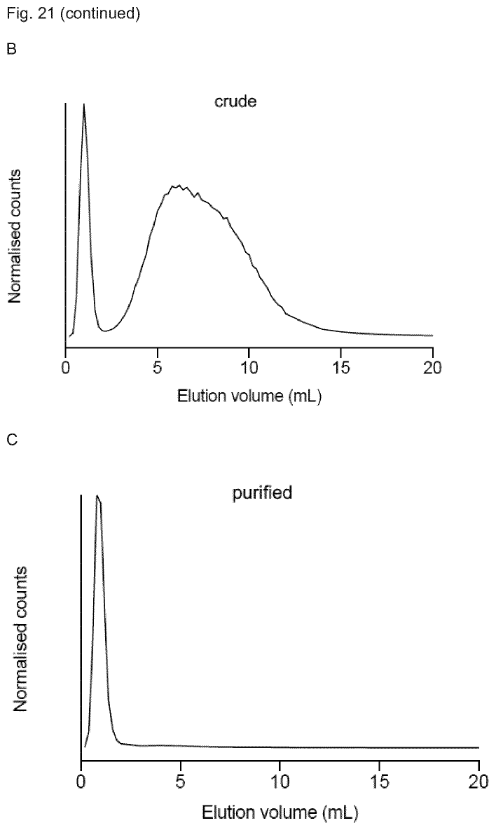

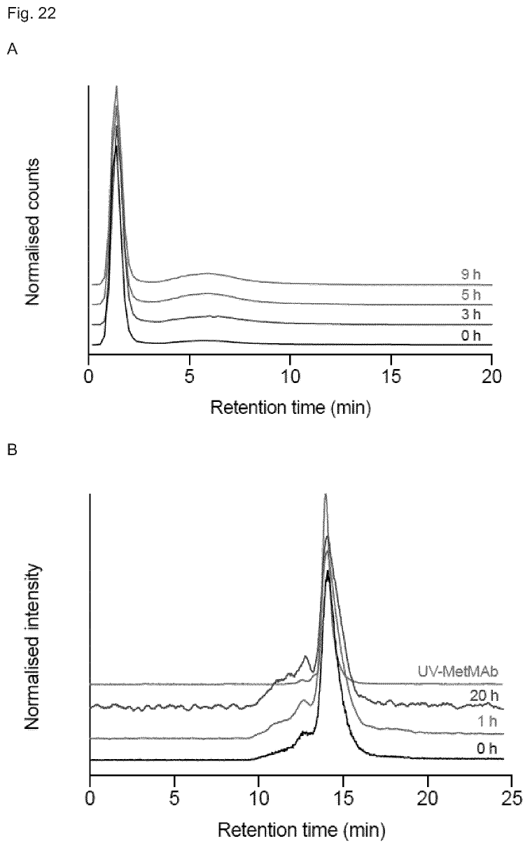

The number of chemically accessible chelates per antibody produced after photochemically conjugating the monoclonal antibody with different initial chelate-to-antibody ratios were estimated by radiolabelling the DFO-azepin-antibody samples using an excess of [⁸⁹Zr][Zr(C₂O₄)₄]⁴⁻(aq.), ensuring that the RCC was <100%. Samples of the crude radiolabelling reactions forming [⁸⁹Zr]ZrDFO-azepin-antibody were analysed by radio-ITLC eluting with EDTA. The fraction of ⁸⁹Zr radioactivity retained at the baseline (R_{f}=0.0) and at the solvent front (R_{f} =1.0, [⁸⁹Zr]ZrEDTA) was determined by integration after appropriate background corrections. Radio-ITLC data for the reaction using the 26.4-fold initial chelate-to-mAb ratio is shown in FIG. 6A and a plot of the RCC/% versus time for reactions using different initial chelate-to-mAb ratios is presented in FIG. 6B. After allowing sufficient time for saturation of the accessible chelates (180 min.), the final RCC was used to estimate the number of accessible chelates per antibody using the measured (decay corrected) molar activity of [⁸⁹Zr][Zr(C₂O₄)₄]⁴⁻ and the known number of moles of antibody added to each reaction. Note that it was assumed that Zr⁴⁺ ions form a 1:1 stoichiometric complex with DFO. The measured accessible chelate-to-mAb ratios were 0.27, 0.55 and 0.85 for DFO-azepin-antibody samples prepared at initial chelate-to-mAb ratios of 5.3, 10.7 and 26.4, respectively. Linear regression analysis indicated that the quantum yield for photochemical coupling of compound 1 with the monoclonal antibody was ˜0.035. The relatively low efficiency is likely due to intramolecular reactions between the activated nitrene, benzazirine or ketenimines intermediates are nucleophilic groups (like hydroxamate anions) in the structure of DFO.

Simultaneous, One-Pot Photoradiochemical Synthesis of [⁸⁹Zr]ZrDFO-azepin-antibody

Simultaneous, one-pot photochemical conjugation and radiolabelling reactions were performed in accordance with the following general procedure. A stock solution of DFO-ArN₃ (1, 0.68 mg, 0.964 μmol) was dissolved in H₂O (50 μL) and NaOH(aq.) (30 μμL of a 0.1 M stock solution). The pH of the DFO-ArN₃ solution was reduced to ˜8-9 by the addition of HCl(aq.) (2×10 μL of a 0.1 M stock solution). Different reactions and control were performed at the same time using the same stock solutions. Details are given in Table S3 below. Details for reaction 1 are given here. To a transparent glass vial containing water (50 μL) was added an aliquot of pre-purified antibody stock solution (stock concentration=23.0 mg/mL, 50 μL added, 1.15 mg of protein, 7.69 nmol), an aliquot of the DFO-ArN₃ stock solution (1, 23 μL, 0.222 μmol, ˜28.9-fold excess) and an aliquot of pre-neutralised [⁸⁹Zr][Zr(C₂O₄)₄]⁴⁻ (aq.) stock solution (50 μL, 4.2 MBq). Note: see the radiochemistry sections above for details about neutralisation of oxalic acid in the ⁸⁹Zr stock solution. The total reaction volume was kept constant at 150 μL for all reactions. Reactions were stirred and irradiated at room temperature for 10 min. at the specified LED wavelength (100% power). Reactions were then quenched by the addition of DTPA (10 μL, 50 mM) and aliquots of the crude reaction mixtures were analysed by using radio-ITLC, analytical PD-10-SEC and SEC-UHPLC. Data are presented in FIG. 7. For reaction 1, an aliquot of the crude, quenched mixture was also purified by preparative PD-10-SEC and spin column centrifugation. After isolation of purified [⁸⁹Zr]ZrDFO-azepin-antibody by preparative PD-10-SEC (collecting the 0.0-2.0 mL high molecular weight fraction using sterile PBS as an eluent) the decay corrected radiochemical yield was ˜76% and the estimated lower limit (assuming no protein losses) of the molar activity was 0.41 MBq/nmol of protein. Aliquots of the purified sample of reaction 1 were then concentrated and analysed by analytical PD-10-SEC and SEC-UHPLC.

TABLE S3
Experimental data on the conditions used in the
simultaneous photoradiolabelling reactions for the synthesis
of [⁸⁹Zr]ZrDFO-azepin-antibody.

| | | Reaction 2 | Reaction 3 | |
|---|---|---|---|---|
| | | (no chelate | (no antibody | |
| Solution | Reaction 1 | control) | control) | Reaction 4 |
| Vol. water/μL | 27 | 50 | 27 | 27 |
| Vol. DFO-ArN₃ | 23 | 0 | 23 | 23 |
| stock/μL | | | | |
| Vol. trastuzumab | 50 | 50 | 0 | 50 |
| stock/μL | | | | |
| Vol. | 50 | 50 | 50 | 50 |
| [⁸⁹Zr]Zr(C₂O₄)₄]⁴⁻ | | | | |
| stock/μL | | | | |
| Total volume/μL | 150 | 150 | 150 | 150 |
| Activity | ~4.2 MBq | ~4.2 MBq | ~4.2 MBq | ~4.2 MBq |
| Irradiation λ/nm | 365 | 365 | 365 | 395 |
| Irradiation time/ | 10 | 10 | 10 | 10 |
| min. | | | | |

### REFERENCES

[1] N. Srinivas, P. Jetter, B. J. Ueberbacher, M. Werneburg, K. Zerbe, J. Steinmann, B. Van Der Meijden, F. Bernardini, A. Lederer, R. L. A. Dias, et al., Science (80-.). 2010, 327, 1010-1013.

[2] P. Zanzonico, J. Nucl. Med. 2008, 49, 1114-1131.

[3] T. Lindmo, E. Boven, F. Cuttitta, J. Fedorko, P. A. Bunn, J. Immunol. Methods 1984, 72, 77-89.

[4] Institute for Laboratory Animal Research, Guide for the Care and Use of Laboratory Animals: 8th Ed., 2011.

[5] R. Fridman, G. Benton, I. Aranoutova, H. K. Kleinman, R. D. Bonfil, Nat. Protoc. 2012, 7, 1138-1144.

[6] B. Bai, M. Dahlbom, R. Park, L. Hughes, G. Dagliyan, L. P. Yap, P. S. Conti, IEEE Nucl. Sci. Symp. Conf. Rec. 2012, bai, 3765-3768.

[31] M. J. W. D. Vosjan, L. R. Perk, G. W. M. Visser, M. Budde, P. Jurek, G. E. Kiefer, G. A. M. S. van Dongen, Nat. Protoc. 2010, 5, 739-743.

[33] J. P. Holland, V. Divilov, N. H. Bander, P. M. Smith-Jones, S. M. Larson, J. S. Lewis, J. Nucl. Med. 2010, 51, 1293-1300.

[34] J. P. Holland, E. Caldas-Lopes, V. Divilov, V. a Longo, T. Taldone, D. Zatorska, G. Chiosis, J. S. Lewis, PLoS One 2010, 5, e8859.

[35] J. P. Holland, M. J. Evans, S. L. Rice, J. Wongvipat, C. L. Sawyers, J. S. Lewis, Nat. Med. 2012, 18, 1586-1591.

[36] S. N. Rylova, L. Del Pozzo, C. Klingeberg, R. Tonnesmann, A. L. Illert, P. T. Meyer, H. R. Maecke, J. P. Holland, J Nucl Med 2016, 57, 96-102.

1. A method for preparing a photoradiolabelled compound comprising
i. providing a reaction mixture comprising
at least one chelating compound, and
at least one target compound B comprising an amine and/or thiol and/or carboxylate moiety, particularly an amine and/or thiol moiety, and
at least one radioactive ion of a radionuclide,
ii. performing photoconjugation and radiolabelling in a photoradiolabelling step by
adjusting the pH to pH>7, in particular pH>8, more particularly pH 8 to 11
irradiation of the reaction mixture with light at a wavelength selected from 200 nm to 420 nm,
wherein the chelating compound is a compound of formula 1, 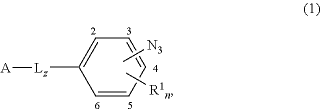
wherein
A is a chelator suitable for coordinating an ion of a radionuclide at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂—CHF₂, —SOCF₃, —SO₂CF₃, —SC₂—NR²R³, —CN, —NO₂, —F, —Cl, —Br or —I, in particular —OH, —CN, —NO₂, —F, —Cl, —Br, or —I, with
R² and R³ being independently selected from C₁₋₆-alkyl, C₂₋₆-alkenyl and C₂-₆-alkynyl,
⁴ being selected from C₁₋₆-alkyl, C₂₋₆-alkenyl and C₂₋₆-alkynyl which may optionally be substituted with —F, —Cl, —Br or —I
n is 0, 1, 2 or 3, in particular 0 or 1, more particularly 0, and
R¹ and —N₃ are positioned in such a way that at least one of the positions 2 to 6 of the phenyl moiety that are next to —N₃ is unsubstituted.

2. The method according to any one of the preceding claims, wherein the radionuclide is selected from ⁴³Sc, ⁴⁴Sc, ⁴⁷Sc, ⁴⁵Ti, ⁵¹Cr, ⁵¹Mn, ⁵²Mn, ^{52m}Mn, ⁵²Fe, ⁵⁵Co, ⁵⁷Ni, ⁶⁰Cu, 61_{Cu,} ⁶²Cu, ⁶⁴Cu, ⁶⁷Cu, ⁶⁵Zn, ⁶⁶Ga, ⁶⁷Ga, ⁶⁸Ga, ⁶⁹Ge, ⁷¹As, ⁷²As, ⁷⁴As, ⁷⁶As, ⁷⁷As, ⁸²Rb, ^{82m}Rb, ⁸²Sr, ⁸³_{Sr,} ⁸⁹_{Sr,} ⁸⁶Y, ⁹⁰Y, ⁸⁹_{Zr}, ⁹⁷Zr, ⁹⁰Nb, ^{94m}Tc, ^{99m}Tc, ⁹⁷Ru, ¹⁰⁵Rh, ¹¹¹Ag, ^{110m}In, ¹¹¹In, ^{117m}Sn, ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, ¹⁷⁸Ta, ¹⁸⁶Re, ¹⁸⁸Re, ¹⁹²Ir, ^{195m}Pt, ¹⁹⁸Au, ^{197m}Hg, ²⁰¹Tl, ²¹²Pb, ²¹²Bi, ²¹³_{Bi,} ²¹¹At, ²²³Ra, ²⁵⁵Ac, in particular from ¹⁵³Sc, ⁴⁴Sc, ⁴⁷Sc, ⁶⁰Cu, ⁶¹Cu, ⁶²Cu, ⁶⁴Cu, ⁶⁷Cu, ⁶⁷Ga, ⁶⁸Ga, ⁸⁶Y, ⁹⁰Y, ⁸⁹Zr, ^{99m}Tc, ¹¹¹In, ¹⁵³Sm, ¹⁴⁹Tb, ¹⁵²Tb, ¹⁵⁵Tb, ¹⁶¹Tb, ⁷⁷Lu, ¹⁸⁶Re, ¹⁸⁸Re, ²¹²Pb, ²¹²Bi, ²¹³Bi, ²²³Ra, ²⁵⁵Ac.

3. The method according to any one of the preceding claims, wherein a co-ligand is added to the reaction mixture, in particular acetate, oxalate or chloride.

4. A chelating compound comprising formula 2, 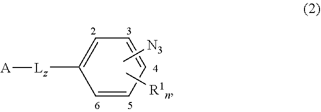
wherein
A is a chelator suitable for coordinating an ion of a radionuclide, particularly at basic pH,
L is a linker with z being 0 or 1,
R¹ is independently from each other selected from C₁₋₆-alkyl, C₂₋₆-alkenyl, C₂₋₆-alkynyl, —NH₂, —NHR², —NR²R³, —OH, —OR⁴, —SR⁴, —CF₃, —CH₂F, —CHF₂, —CH₂—CF₃, —CH₂—CH₂F, —CH₂—CHF₂, —SOCF₃, —SC₂CF₃, —SC₂—NR²R³, —CN, —NO₂, —F, —Cl, —Br or —I, in particular ——OH, —OR⁴, —CN, —NO₂, —F, —Cl, —Br, or —I, with
R² and R³ being independently selected from C₁₋₆-alkyl, C₂₋₆-alkenyl and C₂-₆-alkynyl,
R⁴ being selected from C₁₋₆-alkyl, C₂₋₆-alkenyl and C₂₋₆-alkynyl which may optionally be substituted with —F, —Cl, —Br or —I,
n is 0, 1, 2 or 3,
wherein R¹ and —N₃ are positioned in such a way that at least one of the positions 2 to 6 of the phenyl moiety that are next to —N₃ is unsubstituted,
with the proviso that in case of z being 0, A is not EDTA, and
with the proviso that in case of z being 1, A is not DTPA.

5. A radiolabelled intermediate compound comprising formula 3, 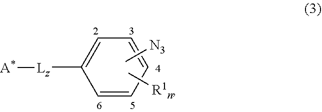
wherein
A* is a chelator bound to a radionuclide by coordinate bonds, and
L, z, R¹ and n are defined as described above.

6. The compound according to any one of claim 1, 4 or 5, wherein —N₃ is in meta or para position, particularly in para position.

7. A photoconjugated intermediate compound comprising formula 4a, 4b, 4c, 4d or 4e, 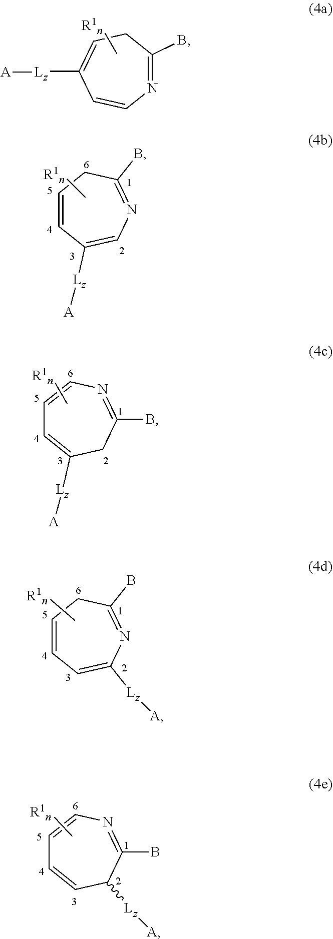
wherein
A, L, z, R¹, n and B are defined as described above.

8. A photoradiolabelled compound comprising formula 5a, 5b, 5c, 5d or 5e, 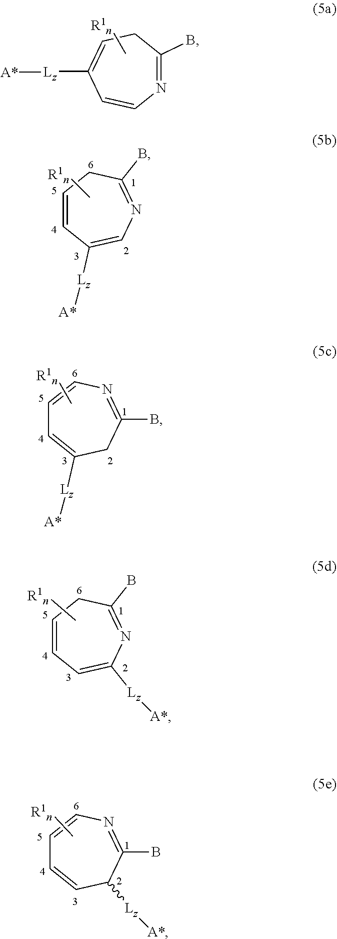
wherein
A*, L, z, R¹, n and B are defined as described above.

9. The compound according to any one of the preceding claims, wherein the chelator is selected from NODAGA, NOTA, DOTA, Desferrioxamine B (DFO), ATSM, DOTAGA, HBED-CC, SAAC, DTPA, DTPA-benzyl, DFO-Star, oxoDFO-Star, HOPO, p-SCN-Bn-HOPO 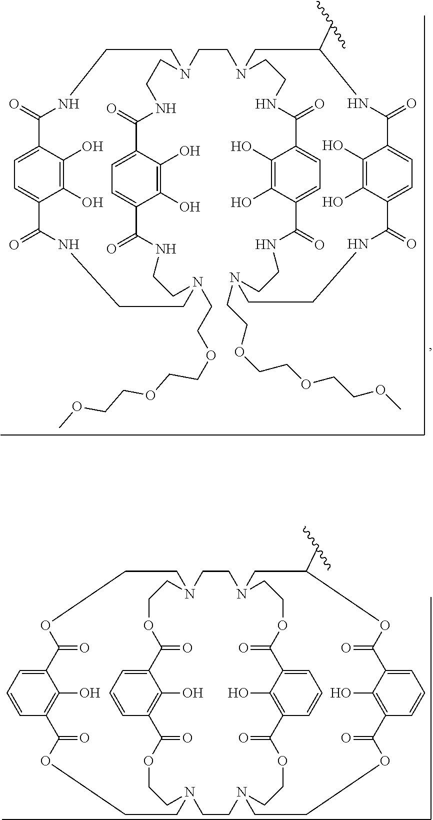 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]
and derivatives thereof, in particular from NODAGA, NOTA, Desferrioxamine B (DFO), ATSM, DOTAGA, HBED-CC, SAAC, DFO-Star, oxoDFO-Star, p-SCN-Bn-HOPO, 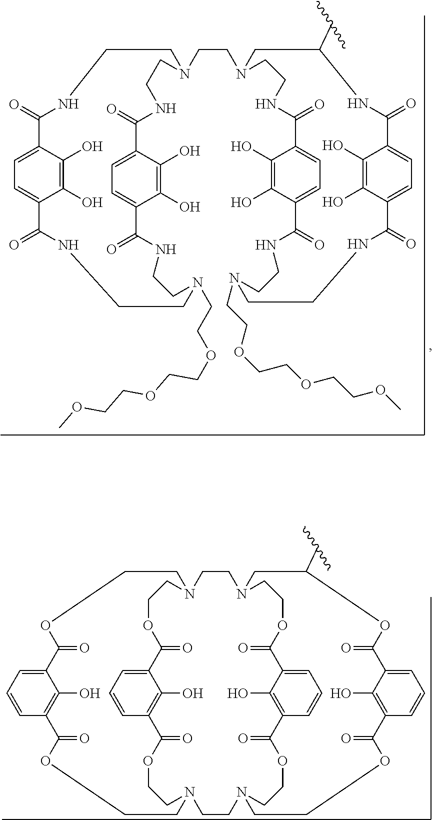 [SMILES: *C1CN(CCN2CCNC(=O)c3ccc(c(O)c3O)C(=O)NCCN(CCOCCOCCOC)CCNC(=O)c3ccc(c(O)c3O)C(=O)NCC2)CCNC(=O)c2ccc(c(O)c2O)C(=O)NCCN(CCOCCOCCOC)CCNC(=O)c2ccc(c(O)c2O)C(=O)N1 ; *C1CN2CCOC(=O)c3cccc(c3O)C(=O)OCCN(CCOC(=O)c3cccc(c3O)C(=O)O1)CCN1CCOC(=O)c3cccc(c3O)C(=O)OCCN(CCOC(=O)c3cccc(c3O)C(=O)OCC1)CC2]
and derivatives thereof.

10. The compound according to any one of the preceding claims, wherein L is a linker comprising one or more moieties, particularly 1 to 20 moieties, more particularly 1 to 15 moieties, selected from —C(═X)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, —O—, —C₁₋₈-alkyl-, particularly selected from —C(═X)—, —NR⁶—, —C(═X)—NR⁶—, —NR⁶—C(═X)—, —NR⁶—C(═X)—NR⁶—, —O—, —C₁₋₈-alkyl-,
with R⁶ being H or C₁₋₈-alkyl and X being O or S.

11. The compound according to any one of the preceding claims, wherein L is —C(═O)— or L comprises one or more moieties selected from —C(═X)—, —NR⁶—, —C(═X)—NR⁶—, —NR⁶—C(═X)—, —NR⁶—C(═X)—NR⁶—, —O—, —C₁₋₈-alkyl- with R⁶ being H or C₁₋₈-alkyl and X being O or S, wherein a moiety that comprises a heteroatom N, O or S alternates with an alkyl moiety, wherein in particular one or both ends of the linker are independently formed by a moiety that comprises a heteroatom N, O or S.

12. The compound according to any one of the preceding claims, wherein L is —C(═O)— or a moiety of formula 2,
R^{a}ₙ—(C₁₋₆-alkyl)-R^{b}ₘ—R^{c}— (2), wherein
R^{a} is —C(═O)—, —NR⁶—C(═X)—NR⁶—, or —NR⁶—, particularly —C(═O)— or —NR⁶—, more particularly —NR⁶—, with R⁶ being H or C₁₋₄-alkyl, or R^{a} is a moiety —X¹—C₁₋₈-alkyl —X²— with X¹ and X² being a moiety independently selected from —C(═O)—, —NR⁶—, —C(═X)—NR⁶—, —NR⁶—C(═X)—, —NR⁶—C(═X)—NR⁶—, —O—C(═X)—NR⁶—, —NR⁶—C(═X)—O—, particularly —C(═O)—, —NR⁶—, —C(═O)—NR⁶—, —NR⁶—C(═O)—,
n is 0 or 1,
R^{b} is a polyether moiety with p elements [—O—Cᵤ-alkyl], wherein u is independently selected for each element from an integer between 1 to 4 and p is an integer between 1 and 6,
m is 0 or 1,
R^{c} is —NR⁵—C(═O)—, —NR⁵—C(═X)—NR⁵—, —O—C(═X)—NR⁵—, —NR⁵—C(═X)—O—, wherein
R⁵ is independently from each other H or C₁₋₄-alkyl
X is O or S, particularly S.

13. The compound according to any one of claim 5 or 8, wherein the radionuclide is selected from ⁴³Sc, ⁴⁴Sc, ⁴⁷Sc, ⁴⁵Ti, ⁵¹Cr, ⁵¹Mn, ⁵²Mn, ^{52m}Mn, ⁵²Fe, ⁵⁵Co, ⁵⁷Ni, ⁶⁰Cu, ⁶¹Cu, ⁶²Cu, ⁶⁴Cu, ⁶⁷Cu, ⁶⁵Zn, ⁶⁶Ga, ⁶⁷Ga, ⁶⁸Ga, ⁶⁹Ge, ⁷¹As, ⁷²As, ⁷⁴As, ⁷⁶As, ⁷⁷As, ⁸²Rb, ^{82m}Rb ⁸²Sr, ⁸³Sr, ⁸⁹Sr, ⁸⁶Y, ⁹⁰Y, ⁸⁹Zr, ⁹⁷Zr, ⁹⁰Nb, ^{94m}Tc, ^{99m}Tc, ⁹⁷Ru, ¹⁰⁵Rh, ¹¹¹Ag, ^{110m}In, ¹¹¹In, ^{117m}Sn, ¹⁵³Sm, ¹⁴⁹Tb, ¹⁵²Tb, ¹⁵⁵Tb, ¹⁶¹Tb, ¹⁶⁶Ho, ¹⁶⁵Er, ¹⁷⁷Lu, ¹⁷⁸Ta, ¹⁸⁶Re, ¹⁸⁸Re, ¹⁹²Ir, ^{195m}Pt, ¹⁹⁸Au, ^{197m}Hg, ²⁰¹Tl, ²¹²Pb, ²¹²Bi, ²¹³Bi, ²¹¹At, ²²³Ra, ²⁵⁵Ac, in particular ⁴³Sc, ⁴⁴Sc, ⁴⁷Sc, ⁶⁰Cu, ⁶¹Cu, ⁶²Cu, ⁶⁴Cu, ⁶⁷Cu, ⁶⁷Ga, ⁶⁸Ga, ⁸⁶Y, ⁹⁰Y, ⁸⁹Zr, ^{99m}Tc, ¹¹¹In, ¹⁵³Sm, ¹⁴⁹Tb, ¹⁵²Tb, ¹⁵⁵Tb, ¹⁶¹Tb, ⁷⁷Lu, ¹⁸⁶Re, ¹⁸⁸Re, ²¹²Pb, ²¹²Bi, ²¹³Bi, ²²³Ra, ²⁵⁵Ac. AC.

14. The compound according to any one of claim 1, 7 or 8, wherein the target compound B is selected from a small molecule, a peptide, a protein, an antibody, an antibody-like molecule, an antibody fragment or a nanoparticle.

15. The compound according to any one of claim 7, 8 or 14, wherein the target compound B is bound to the azepine moiety via said amine of the target compound B or a thioether moiety —S— derived from the thiol moiety —SH of the target compound B, in particular an amine —NH— derived from lysine.
